(19) United States
(12) Patent Application Publication
(10) Pub. No.: US 2025/0280726 A1
(43) Pub. Date: Sep. 4, 2025

(54) ORGANOMETALLIC COMPLEX AND ORGANIC LIGHT-EMITTING ELEMENT

(71) Applicant: CANON KABUSHIKI KAISHA, Tokyo (JP)

(72) Inventors: KATSUYUKI HOTTA, Kanagawa (JP); Tomokazu Tsuji, Fukui (JP); YUTO ITO, Tokyo (JP); KENJI YAMADA, Kanagawa (JP)

(21) Appl. No.: 19/209,247

(22) Filed: May 15, 2025

Related U.S. Application Data
(63) Continuation of application No. PCT/JP2023/039563, filed on Nov. 2, 2023.

(30) Foreign Application Priority Data
Nov. 17, 2022 (JP) 2022-183915

(51) Int. Cl. H10K 85/30; C07F 15/00; C09K 11/02; C09K 11/06; H10K 50/11; H10K 85/40

(52) U.S. Cl. CPC H10K 85/342; C07F 15/0033; C09K 11/02; C09K 11/06; H10K 85/361; H10K 85/40; C09K 2211/1029; C09K 2211/1033; C09K 2211/1037; C09K 2211/104; C09K 2211/1044; C09K 2211/185; H10K 50/11

(57) ABSTRACT

An organic light-emitting element including an organic compound layer containing an organometallic complex that includes a ligand in which a naphthoisoquinoline ring and a naphthalene ring are bonded in a cyclic manner and thus has a small full width at half maximum and high light-emission efficiency.

### CROSS-REFERENCE TO RELATED APPLICATIONS

This application is a Continuation of International Patent Application No. PCT/JP2023/039563, filed Nov. 2, 2023, which claims the benefit of Japanese Patent Application No. 2022-183915, filed Nov. 17, 2022, both of which are hereby incorporated by reference herein in their entirety.

### TECHNICAL FIELD

The present invention relates to an organometallic complex that has an emission spectrum with a small full width at half maximum (FWHM) and that exhibits high light-emission efficiency, an organic light-emitting element including an organic compound layer containing the organometallic complex, and an apparatus and device including the organic light-emitting element.

### BACKGROUND ART

An organic light-emitting element (also referred to as an organic electroluminescent element or an organic EL element) is an electronic element including a first electrode, a second electrode, and an organic compound layer disposed between the electrodes. By injecting electrons and holes through the pair of electrodes, excitons of a luminescent organic compound in the organic compound layer are generated. The organic light-emitting element emits light when the excitons return to their ground state. Recent progress in organic light-emitting elements has been noticeable. For example, low driving voltages, various emission wavelengths, high-speed response, and thinner and lighter light-emitting devices have been enabled.

Currently, the use of phosphorescence has been proposed as an attempt to improve the light-emission efficiency of organic EL elements. Organic EL elements utilizing phosphorescence are expected to have improved light-emission efficiency theoretically about four times the light-emission efficiency of those utilizing fluorescence. Thus, phosphorescent organometallic complexes have been actively created to date. Iridium complexes are representative of phosphorescent organometallic complexes but have a problem in that they generally have a phosphorescence emission spectrum with a broad FWHM. In recent years, attempts have been made to achieve a smaller FWHM, but it is known that it is difficult to simultaneously achieve an improvement in color purity and an improvement in light-emission efficiency by a smaller FWHM. PTL 1 describes the following compound A as a phosphorescent material having high light-emission efficiency, and PTL 2 describes the following compound B as a compound having an emission spectrum with a small peak width.

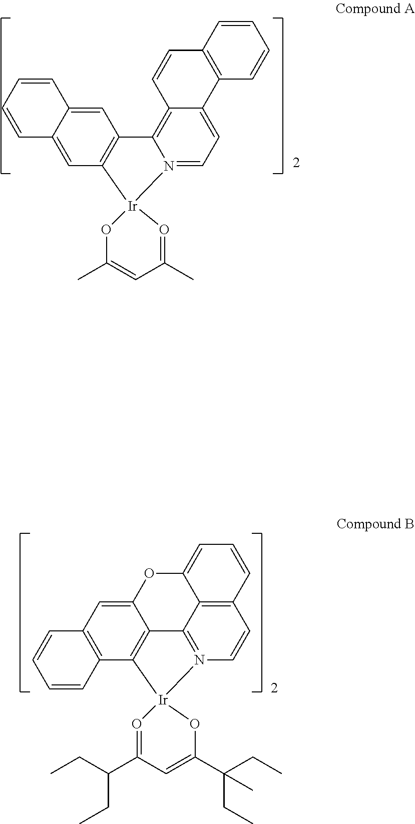

### CITATION LIST

### Patent Literature

PTL 1: Japanese Patent Laid-Open No. 2009-114137
PTL 2: Japanese Patent Laid-Open No. 2021-176839

Compound A described in PTL 1 is a compound having high light-emission efficiency, but has an emission spectrum with a large FWHM and is insufficient in color purity. Compound B described in PTL 2 has an emission spectrum with a small FWHM, but is insufficient in light-emission efficiency.

It is known that a phosphorescent material having a steeper emission spectrum with a smaller FWHM has lower light-emission efficiency, and there has been a need for an organometallic complex having an emission spectrum with a small FWHM and having high light-emission efficiency.

### SUMMARY OF INVENTION

The present invention has been made in view of the above problems, and an object thereof is to provide an organometallic complex having an emission spectrum with a small FWHM and having high light-emission efficiency.

The present invention is an organometallic complex represented by general formula [1] below.

MLₘL′ₙL″ₚ [1]

In general formula [1], M is a transition metal, and L, L′, and L″ represent ligands that are different from each other. m is an integer of 1 to 3, n is an integer of 0 to 2, and p is an integer of 0 to 2, provided that m+n+p=3. MLₘ is represented by general formula [2] below.

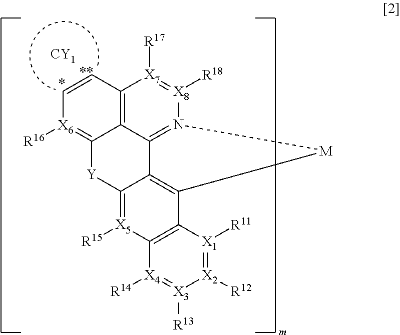

In general formula [2], R₁₁ to R₁₈ are each independently selected from a hydrogen atom, a deuterium atom, a halogen atom, a substituted or unsubstituted alkyl group, a substituted or unsubstituted alkoxy group, a substituted or unsubstituted aryl group, a substituted or unsubstituted aryloxy group, a substituted or unsubstituted heterocyclic group, a substituted or unsubstituted aralkyl group, a substituted or unsubstituted amino group, a silyl group, and a cyano group.

Y is selected from CRR′, SiRR′, S, SO, SO₂, NR, O, Se, PRR′, PO, and SeO₂, where R and R′ are each independently selected from a substituted or unsubstituted alkyl group, a substituted or unsubstituted aryl group, and a halogen atom.

X₁ to X₈ are each independently selected from a carbon atom and a nitrogen atom, and when X₁ to X₈ are carbon atoms, the carbon atoms may be substituted with an alkyl group or an aryl group. When two adjacent X's among X₁ to X₈ are carbon atoms, the two adjacent carbon atoms may be bonded to each other to form a ring structure.

* and ** represent a position of bonding to a ring structure CY₁. CY₁ is represented by any of general formulas [3-1] to [3-3] below.

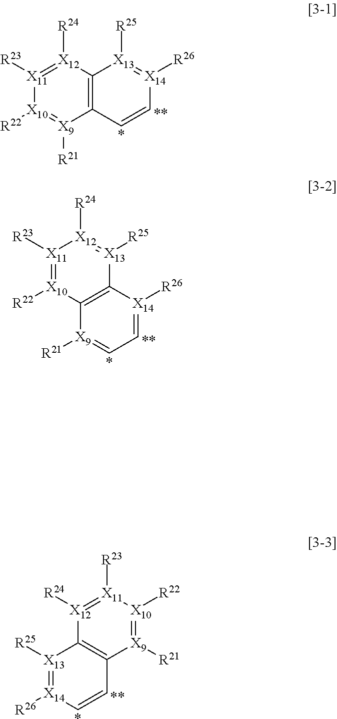

In general formulas [3-1] to [3-3], R₂₁ to R₂₆ are each independently selected from a hydrogen atom, a deuterium atom, a halogen atom, a substituted or unsubstituted alkyl group, a substituted or unsubstituted alkoxy group, a substituted or unsubstituted aryl group, a substituted or unsubstituted aryloxy group, a substituted or unsubstituted heterocyclic group, a substituted or unsubstituted aralkyl group, a substituted or unsubstituted amino group, a silyl group, and a cyano group.

X₉ to X₁₄ are each independently selected from a carbon atom and a nitrogen atom, and when X₉ to X₁₄ are carbon atoms, the carbon atoms may be substituted with an alkyl group or an aryl group. When two adjacent X's among X₉ to X₁₄ are carbon atoms, the two adjacent carbon atoms may be bonded to each other to form a ring structure.

* and ** represent a position of bonding to an isoquinoline ring.

ML′ₙ and ML″ₚ are each independently selected from general formulas [4] and [5] below.

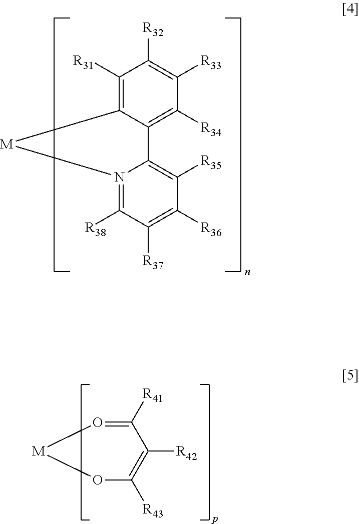

In general formulas [4] and [5], R₃₁ to R₃₈ and R₄₁ to R₄₃ are each independently selected from a hydrogen atom, a deuterium atom, a halogen atom, a substituted or unsubstituted alkyl group, a substituted or unsubstituted alkoxy group, a substituted or unsubstituted aryloxy group, a substituted or unsubstituted aralkyl group, a substituted or unsubstituted amino group, a substituted or unsubstituted aryl group, and a substituted or unsubstituted heterocyclic group.

Further features of the present invention will become apparent from the following description of exemplary embodiments with reference to the attached drawings.

### BRIEF DESCRIPTION OF DRAWINGS

FIG. 1A is a schematic sectional view showing an example of a pixel of a display apparatus according to an embodiment of the present invention.

FIG. 1B is a schematic sectional view of an example of a display apparatus including an organic light-emitting element according to an embodiment of the present invention.

FIG. 2 is a schematic view showing an example of a display apparatus according to an embodiment of the present invention.

FIG. 3A is a schematic view showing an example of an image pickup apparatus according to an embodiment of the present invention.

FIG. 3B is a schematic view showing an example of an electronic device according to an embodiment of the present invention.

FIG. 4A is a schematic view showing an example of a display apparatus according to an embodiment of the present invention.

FIG. 4B is a schematic view showing an example of a foldable display apparatus.

FIG. 5A is a schematic view showing an example of a lighting apparatus according to an embodiment of the present invention.

FIG. 5B is a schematic view showing an example of an automobile including a vehicle lighting fixture according to an embodiment of the present invention.

FIG. 6A is a schematic view showing an example of a wearable device according to an embodiment of the present invention.

FIG. 6B is a schematic view of an example of a wearable device according to an embodiment of the present invention, the wearable device including an image pickup apparatus.

### DESCRIPTION OF EMBODIMENTS

An organometallic complex according to this embodiment is represented by general formula [1] below.

MLₘL′ₙL″ₚ [1]

In the formula [1], M is a transition metal, and L, L′, and L″ represent ligands that are different from each other. [0032]m is an integer of 1 to 3, n is an integer of 0 to 2, and p is an integer of 0 to 2, provided that m+n+p=3.

MLₘ is represented by general formula [2] below.

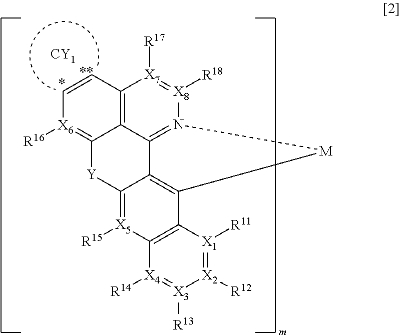

In general formula [2], R₁₁ to R₁₈ are each independently selected from a hydrogen atom, a deuterium atom, a halogen atom, a substituted or unsubstituted alkyl group, a substituted or unsubstituted alkoxy group, a substituted or unsubstituted aryl group, a substituted or unsubstituted aryloxy group, a substituted or unsubstituted heterocyclic group, a substituted or unsubstituted aralkyl group, a substituted or unsubstituted amino group, a silyl group, and a cyano group.

Y is selected from CRR′, SiRR′, S, SO, SO₂, NR, O, Se, PRR′, PO, and SeO₂, where R and R′ are each independently selected from a substituted or unsubstituted alkyl group, a substituted or unsubstituted aryl group, and a halogen atom.

X₁ to X₈ are each independently selected from a carbon atom and a nitrogen atom, and when X₁ to X₈ are carbon atoms, the carbon atoms may be substituted with an alkyl group or an aryl group. When two adjacent X's among X₁ to X₈ are carbon atoms, the two adjacent carbon atoms may be bonded to each other to form a ring structure.

* and ** represent a position of bonding to a ring structure CY₁.

CY₁ is represented by any of general formulas [3-1] to [3-3] below.

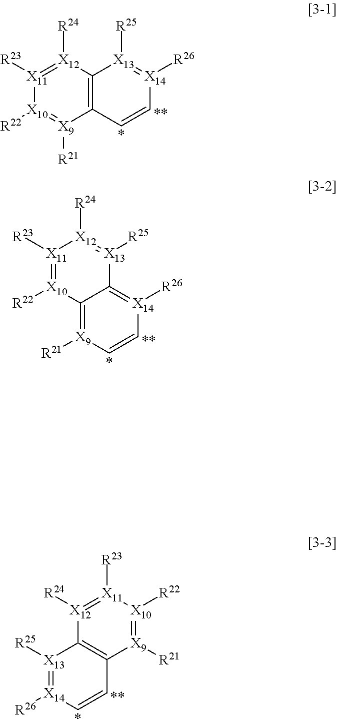

In general formulas [3-1] to [3-3], R₂₁ to R₂₆ are each independently selected from a hydrogen atom, a deuterium atom, a halogen atom, a substituted or unsubstituted alkyl group, a substituted or unsubstituted alkoxy group, a substituted or unsubstituted aryl group, a substituted or unsubstituted aryloxy group, a substituted or unsubstituted heterocyclic group, a substituted or unsubstituted aralkyl group, a substituted or unsubstituted amino group, a silyl group, and a cyano group.

X₉ to X₁₄ are each independently selected from a carbon atom and a nitrogen atom, and when X₉ to X₁₄ are carbon atoms, the carbon atoms may be substituted with an alkyl group or an aryl group. When two adjacent X's among X₉ to X₁₄ are carbon atoms, the two adjacent carbon atoms may be bonded to each other to form a ring structure.

* and ** represent a position of bonding to an isoquinoline ring.

ML′ₙ and ML″ₚ are each independently selected from general formulas [4] and [5] below.

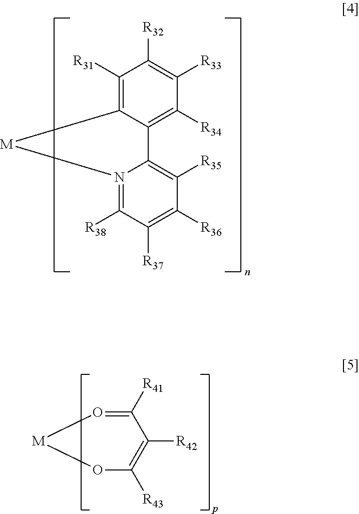

In general formulas [4] and [5], R₃₁ to R₃₈ and R₄₁ to R₄₃ are each independently selected from a hydrogen atom, a deuterium atom, a halogen atom, a substituted or unsubstituted alkyl group, a substituted or unsubstituted alkoxy group, a substituted or unsubstituted aryloxy group, a substituted or unsubstituted aralkyl group, a substituted or unsubstituted amino group, a substituted or unsubstituted aryl group, and a substituted or unsubstituted heterocyclic group.

For X₁ to X₈ in general formula [2] and X₉ to X₁₄ in general formulas [3-1] to [3-3], when two adjacent X's are carbon atoms that are bonded to each other to form a ring structure, the ring may be an alicyclic ring, an aromatic ring, or a heterocyclic ring. The number of fused rings constituting the ring structure may be 1 to 3, and is preferably 1.

When X₅ is a nitrogen atom, the organometallic complex has a deep highest occupied molecular orbital (HOMO), thus having a shorter emission wavelength.

When X₁₁ is a nitrogen atom, the organometallic complex has a low lowest unoccupied molecular orbital (LUMO), thus having a longer emission wavelength.

In general formula [2], a configuration in which CY₁ is represented by general formula [3-1], and Y is selected from CRR′, SiRR′, S, SO, SO₂, NR, O, and Se, where R and R′ are each independently selected from a substituted or unsubstituted alkyl group, a substituted or unsubstituted aryl group, and a halogen atom is preferred. This is because this configuration provides the organometallic complex with higher light-emission efficiency than a configuration in which a ring structure is formed at any other position. In this configuration, X₁ to X₁₄ are preferably each a carbon atom because the organometallic complex has high stability.

In general formula [5], when R₄₁ and R₄₃ are each an alkyl group, the alkyl group is preferably an alkyl group having 1 to 8 carbon atoms, and specific examples include a methyl group, an ethyl group, an isopropyl group, a t-butyl group, and —CH(C₂H₅)₂. When R₄₁ to R₄₃ are bulky substituents, the organometallic complex may have improved heat resistance and sublimability. In particular, the combination of R₄₁, R₄₂, and R₄₃ is preferably a combination of an isopropyl group, a hydrogen atom, and an isopropyl group, a combination of —CH(C₂H₅)₂, a hydrogen atom, and —CH(C₂H₅)₂, a combination of a t-butyl group, a hydrogen atom, and a t-butyl group, or a combination of an ethyl group, a hydrogen atom, and a methyl group.

In this embodiment, the transition metal atom (M in general formula [1]) may be trivalent. Specific examples include Ir, Co, Ru, Os, Rh, and Re, and Ir is preferred.

The halogen atom, the alkyl group, the alkoxy group, the aryl group, the aryloxy group, the heterocyclic group, the aralkyl group, the amino group, and the silyl group given as examples of R₁₁ to R₁₈ in general formula [2], R₂₁ to R₂₆ in general formulas [3-1] to [3-3], R₃₁ to R₃₈ in general formula [4], and R₄₁ to R₄₃ in general formula [5], and substituents that may be substituted on them will be described in detail.

Examples of the halogen atom include fluorine, chlorine, bromine, and iodine, but are not limited thereto. In particular, the halogen atom is preferably a fluorine atom.

The alkyl group may be an alkyl group having 1 to 10 carbon atoms, more preferably 1 to 8 carbon atoms, still more preferably 1 to 4 carbon atoms. Specific examples include a methyl group, an ethyl group, a n-propyl group, an isopropyl group, a n-butyl group, a tert-butyl group, a sec-butyl group, an octyl group, a cyclopentyl group, a cyclohexyl group, a 1-adamantyl group, and a 2-adamantyl group, but are not limited thereto.

The alkoxy group may be an alkoxy group having 1 to 10 carbon atoms, more preferably 1 to 6 carbon atoms, still more preferably 1 to 4 carbon atoms. Specific examples include a methoxy group, an ethoxy group, a propoxy group, a 2-ethyl-hexyloxy group, and a benzyloxy group, but are not limited thereto.

The amino group may be an amino group that is unsubstituted or substituted with any one of an alkyl group, an aryl group, and an amino group. The alkyl group, the aryl group, and the amino group serving as a substituent may be further substituted with a halogen atom, and the aryl group and the amino group may be substituted with an alkyl group. Furthermore, alkyl groups substituted on the amino group may be bonded to each other to form a ring. Specific examples include an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-tert-butylphenyl)amino group, an N-phenyl-N-(4-trifluoromethylphenyl)amino group, and an N-piperidyl group, but are not limited thereto.

The aryl group may be an aryl group having 6 to 18 carbon atoms, and specific examples include a phenyl group, a naphthyl group, an indenyl group, a biphenyl group, a terphenyl group, a fluorenyl group, a phenanthryl group, and a triphenylenyl group.

The heterocyclic group may be a heterocyclic group having 3 to 15 carbon atoms. The heteroatom on the heterocyclic group may be nitrogen, sulfur, or oxygen. Specific examples include a pyridyl group, a pyrazyl group, a pyrimidyl group, a triazyl group, an imidazolyl group, an oxazolyl group, an oxadiazolyl group, a thiazolyl group, a thiadiazolyl group, a carbazolyl group, an acridinyl group, a phenanthrolyl group, a furanyl group, a thiophenyl group, a dibenzofuranyl group, and a dibenzothiophenyl group, but are not limited thereto.

Examples of the aryloxy group include a phenoxy group and a thienyloxy group, but are not limited thereto.

The aralkyl group is an alkyl group substituted with an aryl group, and this alkyl group preferably has 1 to 10 carbon atoms, more preferably has 1 to 8 carbon atoms, and still more preferably has 1 to 4 carbon atoms. Specific examples include a methyl group, an ethyl group, a n-propyl group, an isopropyl group, a n-butyl group, a tert-butyl group, a sec-butyl group, an octyl group, a cyclopentyl group, a cyclohexyl group, a 1-adamantyl group, and a 2-adamantyl group, but are not limited thereto. Furthermore, the aryl group substituted with such an alkyl group may be an aryl group having 6 to 18 carbon atoms, and specific examples include a phenyl group, a naphthyl group, an indenyl group, a biphenyl group, a terphenyl group, a fluorenyl group, a phenanthryl group, and a triphenylenyl group.

Examples of the silyl group include a trimethylsilyl group and a triphenylsilyl group, but are not limited thereto.

The alkyl group, the alkoxy group, the amino group, the aryl group, the aryloxy group, the heterocyclic group, and the aralkyl group mentioned above may be substituted with a deuterium atom, and examples of such an alkyl group whose hydrogen atom is substituted with a deuterium atom include —CD₃, —CD₂CH₃, and —CD₂CD₃, but are not limited thereto.

The alkyl group, the alkoxy group, the amino group, the aryl group, the aryloxy group, the heterocyclic group, and the aralkyl group mentioned above may be substituted with a halogen atom. The halogen atom may be fluorine, chlorine, bromine, or iodine, and is preferably a fluorine atom. In particular, a trifluoromethyl group (—CF₃) and a pentafluoroethyl group (—C₂F₅), which are derived from substitution of hydrogen atoms of the alkyl group with fluorine atoms, are preferred.

The amino group, the aryl group, the aryloxy group, and the heterocyclic group mentioned above may have an alkyl group as a substituent, and this alkyl group preferably has 1 to 10 carbon atoms. Specific examples include a methyl group, an ethyl group, a n-propyl group, an isopropyl group, a n-butyl group, and a t-butyl group.

The alkyl group, the alkoxy group, the amino group, the aryl group, the aryloxy group, the heterocyclic group, and the aralkyl group mentioned above may have, for example, an aryl group, a heterocyclic group, an amino group, an aralkyl group, an alkoxy group, an aryloxy group, or a cyano group as a substituent. The aryl group preferably has 6 to 12 carbon atoms, and specific examples include a phenyl group, a biphenyl group, and a naphthyl group. The heterocyclic group preferably has 3 to 9 carbon atoms, and the heteroatom is preferably nitrogen, sulfur, or oxygen. Specific examples include a pyridyl group and a pyrrolyl group. The amino group may be further substituted with alkyl groups or aryl groups, and the alkyl groups may be bonded to each other to form a ring. Specific examples include a dimethylamino group, a diethylamino group, a dibenzylamino group, a diphenylamino group, and a ditolylamino group. The aralkyl group may be a benzyl group, the alkoxy group may be a methoxy group, an ethoxy group, or a propoxy group, and the aryloxy group may be a phenoxy group.

The organometallic complex according to this embodiment has an emission spectrum with a small full width at half maximum (FWEM) and can provide an organic EL material having high light-emission efficiency. Here, to increase the efficiency of the emission quantum yield of a phosphorescent material such as an iridium complex, it is effective to increase the transition dipole moment of the complex in the excited state and improve the oscillator strength. In an iridium complex having a naphthoisoquinoline (NIQ) ring in this embodiment, the conjugation is extended in such a direction that the center of gravity of a conjugate plane goes away from the metal atom. Accordingly, in the excited state of the complex, the moving distance of an electron from the metal atom to a ligand is increased, so that the transition dipole moment can be increased to improve the oscillator strength. Thus, the organometallic complex has high light-emission efficiency. Here, the FWHM and the oscillator strength of the organometallic complex represented by general formula [1] can be determined by performing structural optimization calculations in the ground state using Gaussian09* Revision C.01, which is electronic state calculation software. For the calculations, the density functional theory was employed as a quantum chemical calculation method, and LC-BLYP was used as the functional. In the case of using Gaussian 09, Revision C.01, 6-31+G** can be used as the basis function to perform the calculations.

Gaussian 09, Revision C.01, M. J. Frisch, G. W. Trucks, H. B. Schlegel, G. E. Scuseria, M. A. Robb, J. R. Cheeseman, G. Scalmani, V. Barone, B. Mennucci, G. A. Petersson, H. Nakatsuji, M. Caricato, X. Li, H. P. Hratchian, A. F. Izmaylov, J. Bloino, G. Zheng, J. L. Sonnenberg, M. Hada, M. Ehara, K. Toyota, R. Fukuda, J. Hasegawa, M. Ishida, T. Nakajima, Y. Honda, O. Kitao, H. Nakai, T. Vreven, J. A. Montgomery, Jr. J. E. Peralta, F. Ogliaro, M. Bearpark, J. J. Heyd, E. Brothers, K. N. Kudin, V. N. Staroverov, T. Keith, R. Kobayashi, J. Normand, K. Raghavachari, A. Rendell, J. C. Burant, S. S. Iyengar, J. Tomasi, M. Cossi, N. Rega, J. M. Millam, M. Klene, J. E. Knox, J. B. Cross, V. Bakken, C. Adamo, J. Jaramillo, R. Gomperts, R. E. Stratmann, O. Yazyev, A. J. Austin, R. Cammi, C. Pomelli, J. W. Ochterski, R. L. Martin, K. Morokuma, V. G. Zakrzewski, G. A. Voth, P. Salvador, J. J. Dannenberg, S. Dapprich, A. D. Daniels, O. Farkas, J. B. Foresman, J. V. Ortiz, J. Cioslowski, and D. J. Fox, Gaussian, Inc. Wallingford CT, 2010.

Here, the oscillator strength and the FWHM of compounds A to F in PTLs 1 and 2 and exemplary compounds 102, 181, and 154 according to this embodiment given later were calculated. The calculation results are shown in Table 1. In Table 1, “n.d.” means “not determined”. Exemplary compound 102 is as follows: in general formula [1], m is 2 and n is 1, in general formula [2], CY₁ is represented by general formula [3-1], R₁₁ to R₁₈ and R₂₁ to R₂₆ are hydrogen atoms, X₁ to X₁₄ are carbon atoms, Y is C(CH₃)₂, M is Ir, and L′ is 3,7-diethylnonane-4,6-dione represented by general formula [5] where R₄₁ and R₄₃ are isopentyl groups and R₄₂ is a hydrogen atom. Exemplary compound 102 is an organometallic complex having two main ligands in each of which a NIQ ring and a naphthyl group bonded to Ir are bonded through C(CH₃)₂ and one ancillary ligand consisting of 3,7-diethylnonane-4,6-dione.

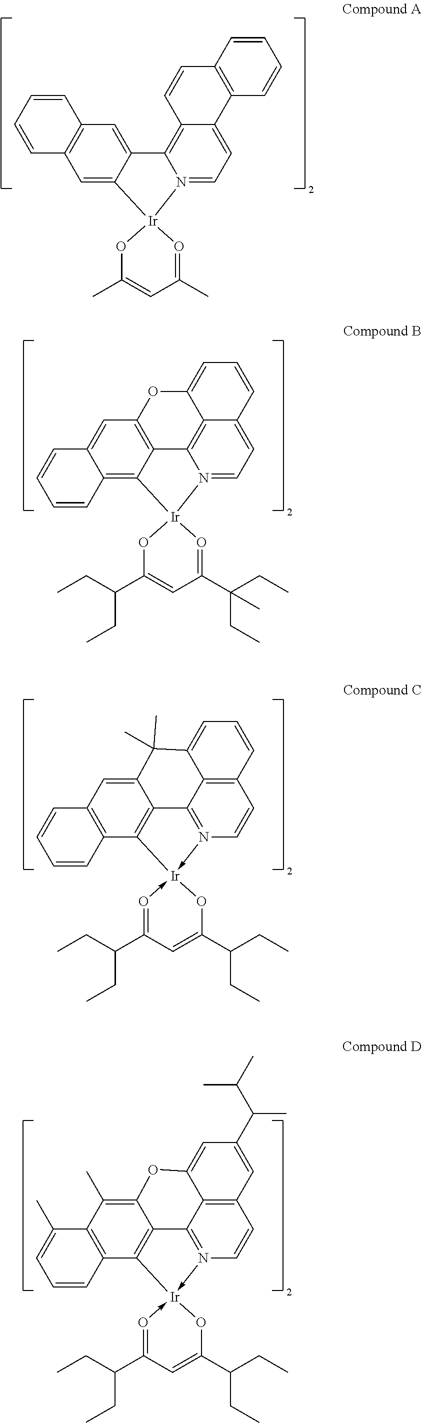

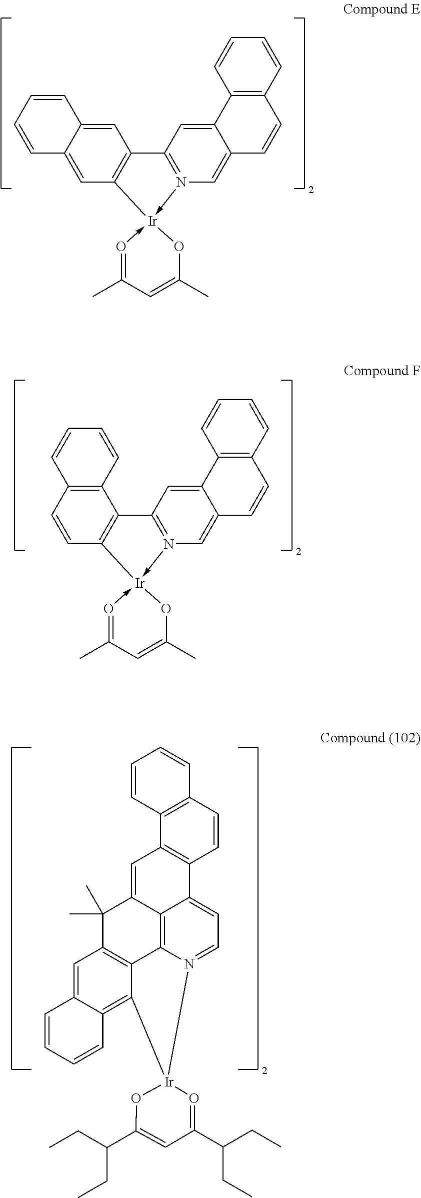

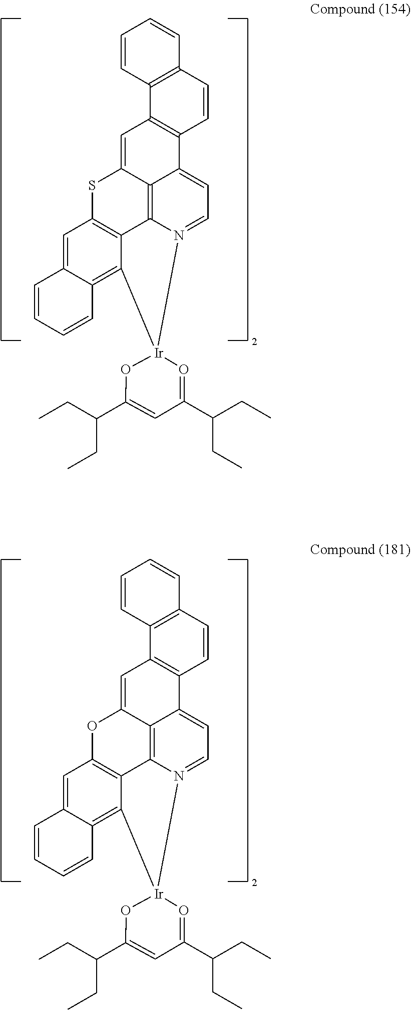

TABLE 1

| | | Oscillator |
|---|---|---|
| Compound | FWHM (nm) | strength |
| Compound A | n.d. | 5.80 × 10⁻⁴ |
| Compound B | 54 | 3.79 × 10⁻⁴ |
| Compound C | 52 | 3.92 × 10⁻⁴ |
| Compound D | 53 | 2.59 × 10⁻⁴ |
| Compound E | 104 | 8.00 × 10⁻⁵ |
| Compound F | n.d. | 1.00 × 10⁻⁴ |
| Exemplary | 46 | 6.20 × 10⁻⁴ |
| compound 102 | | |
| Exemplary | 45 | 1.30 × 10⁻⁴ |
| compound 181 | | |
| Exemplary | 49 | 4.03 × 10⁻⁴ |
| compound 154 | | |

As shown in Table 1, according to the calculations, the oscillator strength of exemplary compound 102 is 1.1 times the oscillator strength of compound A and 1.6 times the oscillator strength of compound B. This shows that the NIQ ring of exemplary compound 102 increases the transition dipole moment of the organometallic complex, and contributes to the increase in oscillator strength and the consequent improvement in light-emission efficiency. Focusing on the FWHM, the FWHM of compound A is not determined by calculations, suggesting a significant structural change between the excited state and the ground state. A broad emission spectrum of an organometallic complex is due to a difference in structure between the ground state and the excited state of the organometallic complex. That is, an organometallic complex with a small difference in structure between the ground state and the excited state has a less broadened emission spectrum and a small FWHM. From this, it can be predicted that compound A has a broad emission spectrum and a large FWHM. By contrast, an iridium complex having a ligand in which a NIQ ring and a naphthalene ring are bonded in a cyclic manner, such as exemplary compound 102, is a compound with a rigid structure, and thus its change in structure between the ground state and the excited state is reduced. Therefore, the organometallic complex according to this embodiment having a cyclic ligand has an emission spectrum with a small FWHM. At this time, the FWHM is preferably 35 nm or less. As compared with compound B, which also has a cyclic ligand, exemplary compound 102 has a more rigid structure due to the NIQ ring and hence a smaller FWHM. Thus, exemplary compound 102 has been found to be an organometallic complex suitable for achieving both high efficiency and high color purity. The calculation results in Table 1 also show that exemplary compound 181 and exemplary compound 154 according to this embodiment each having a NIQ ring have a smaller FWHM than any of compounds A to F.

Specific examples of structural formulas of the organometallic complex according to this embodiment are shown below. However, the organometallic complex according to this embodiment is not limited to these examples.

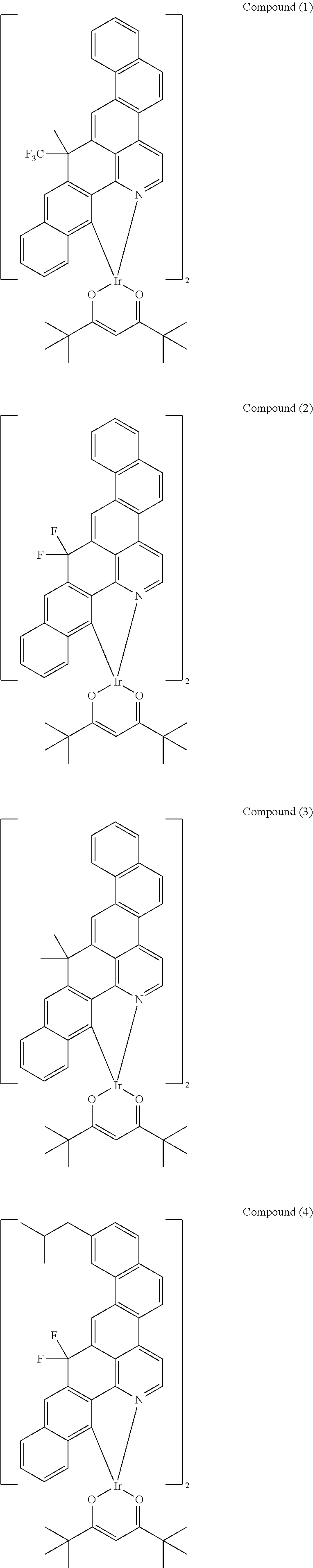

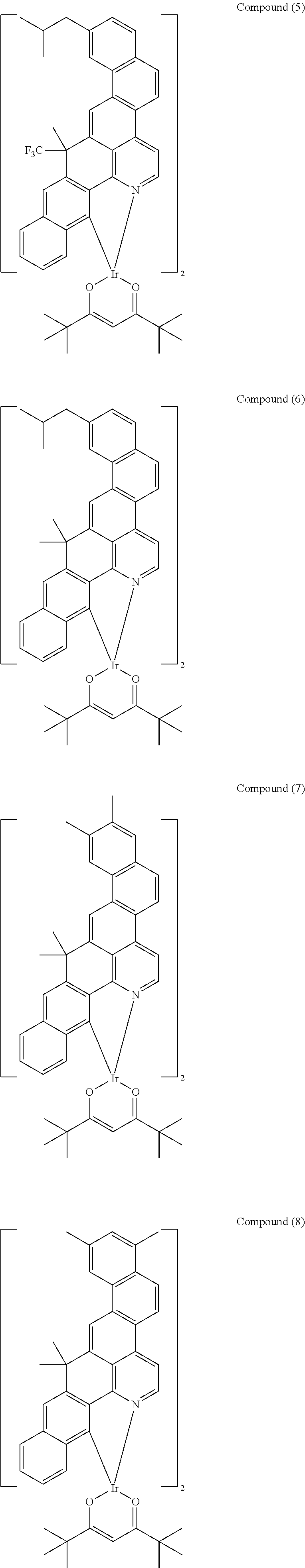

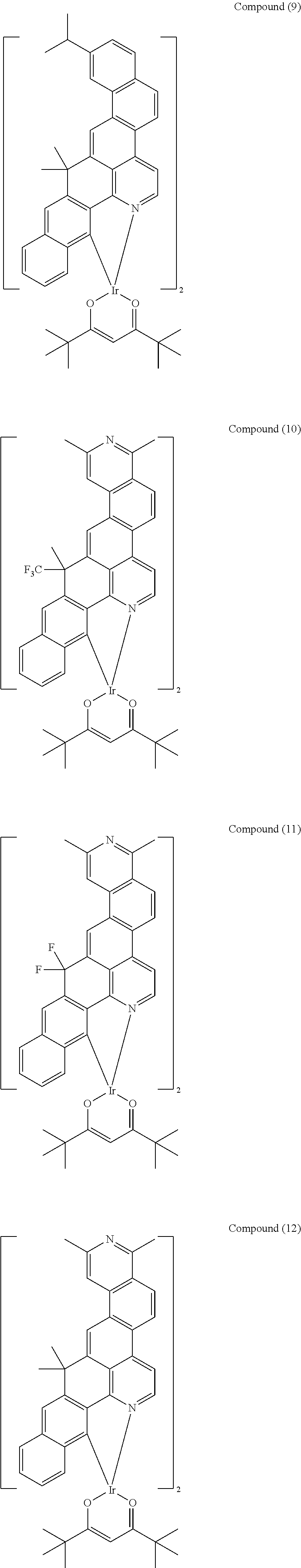

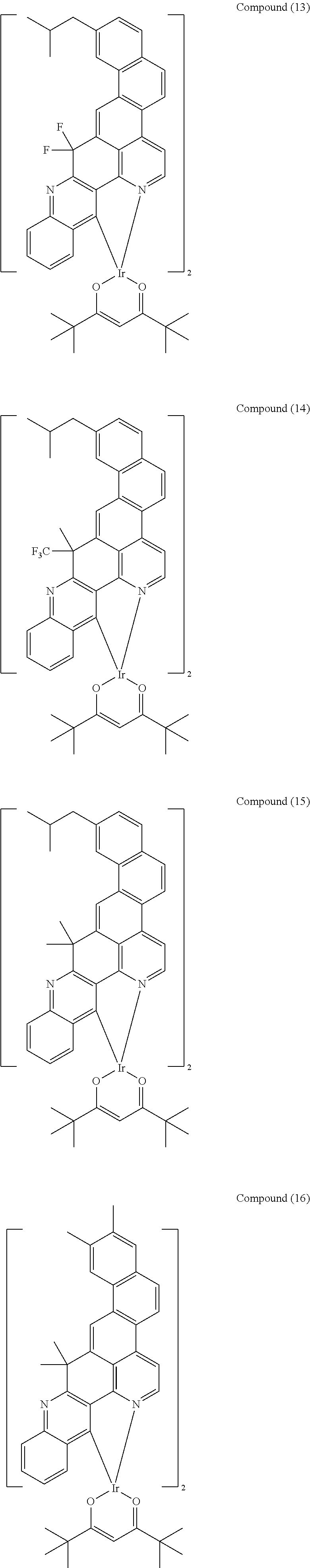

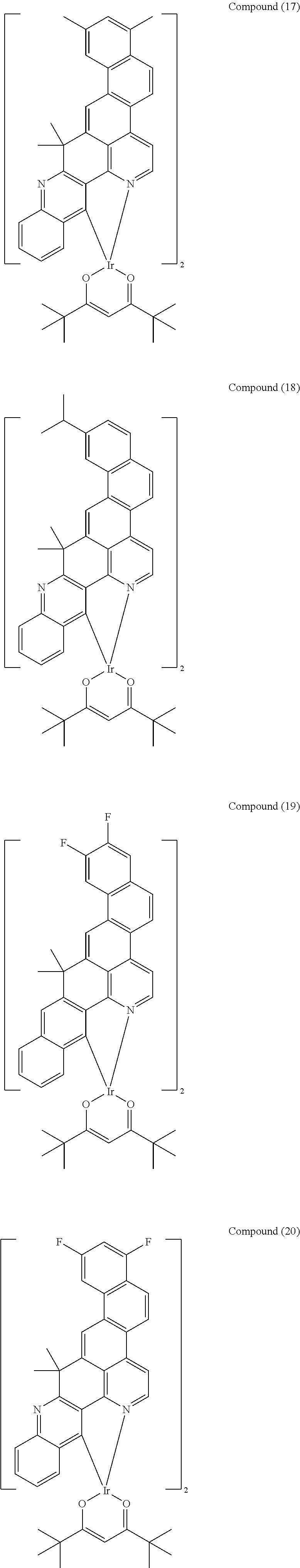

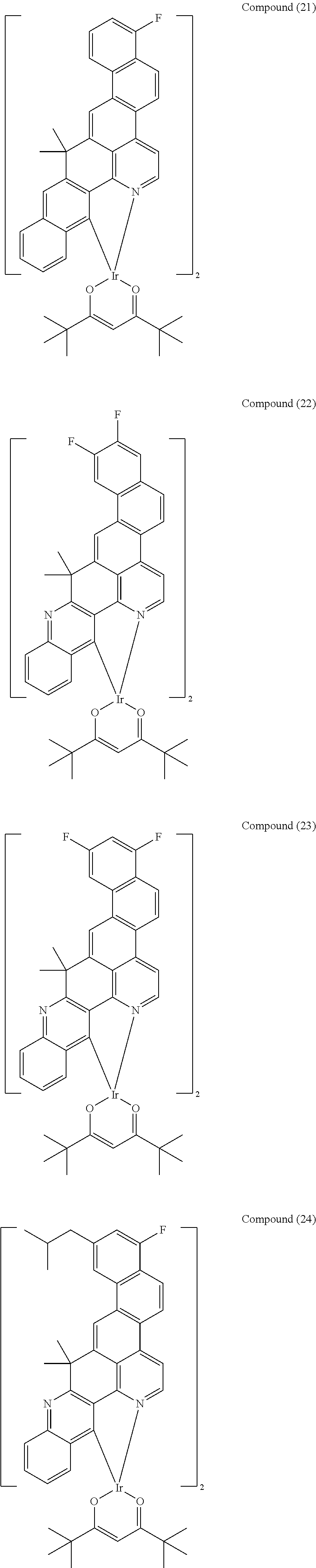

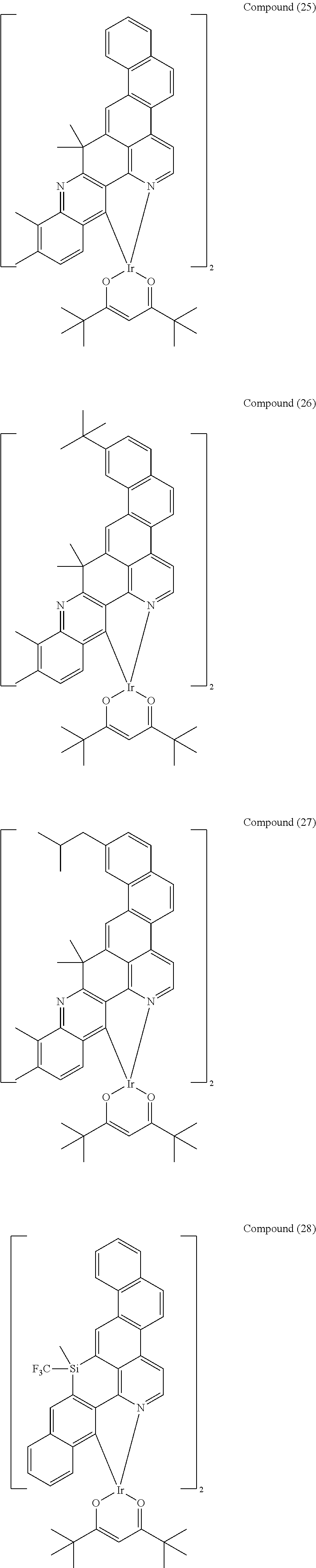

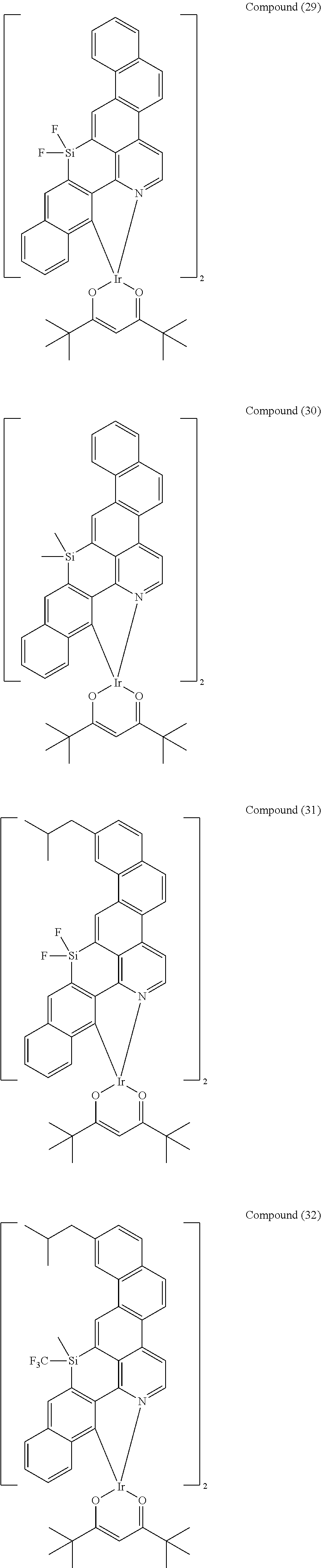

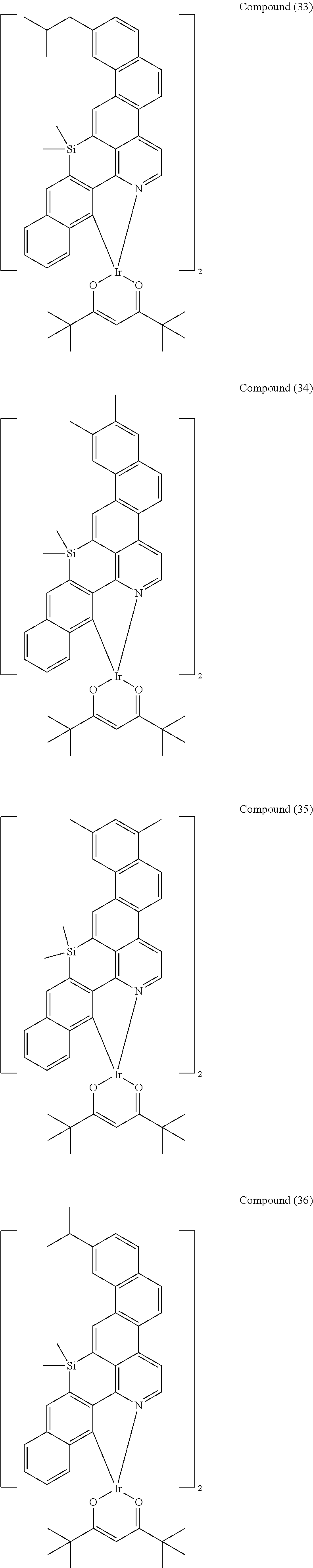

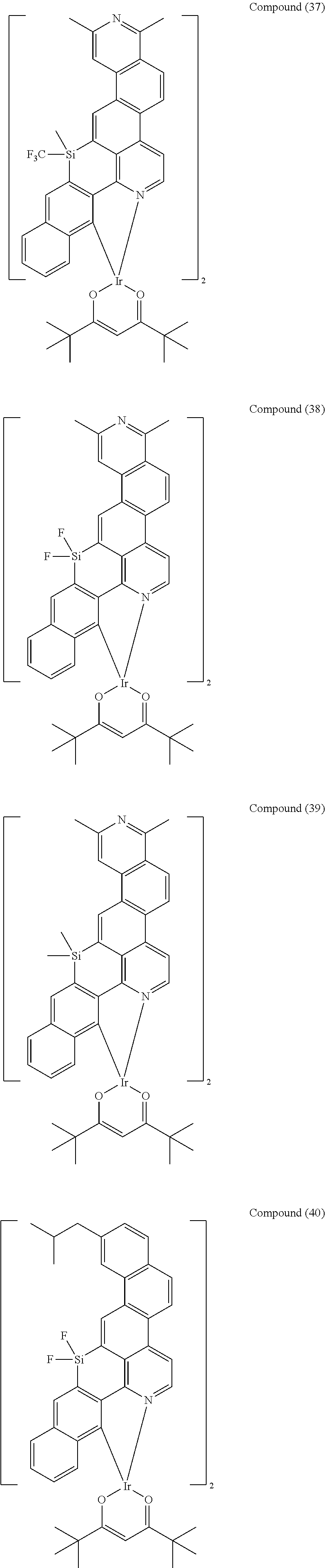

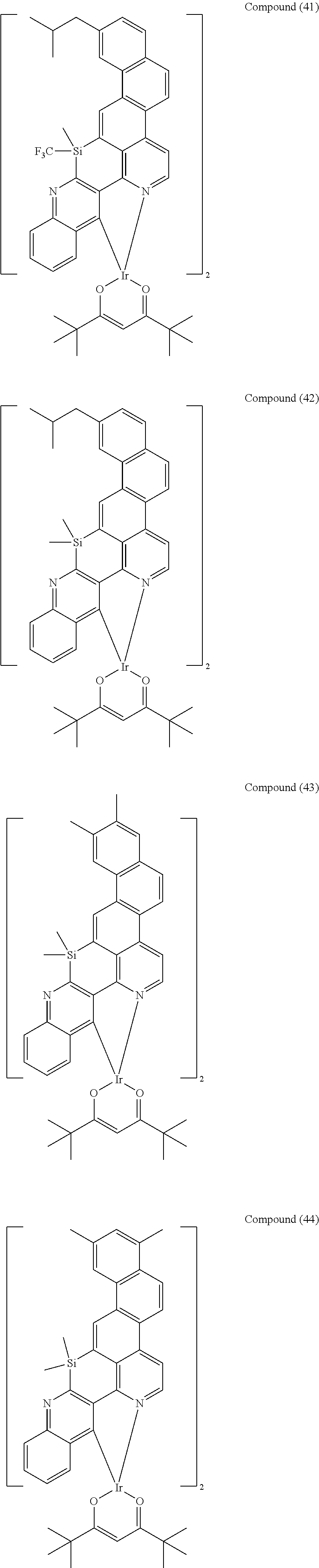

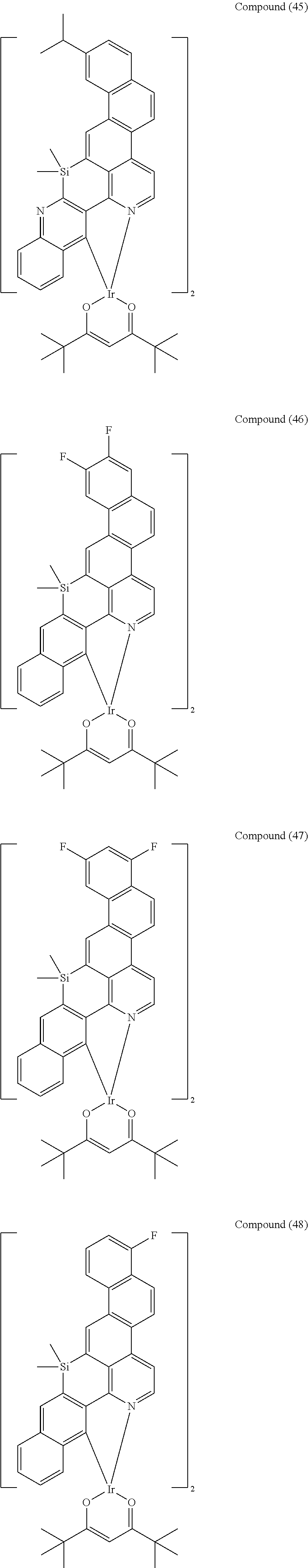

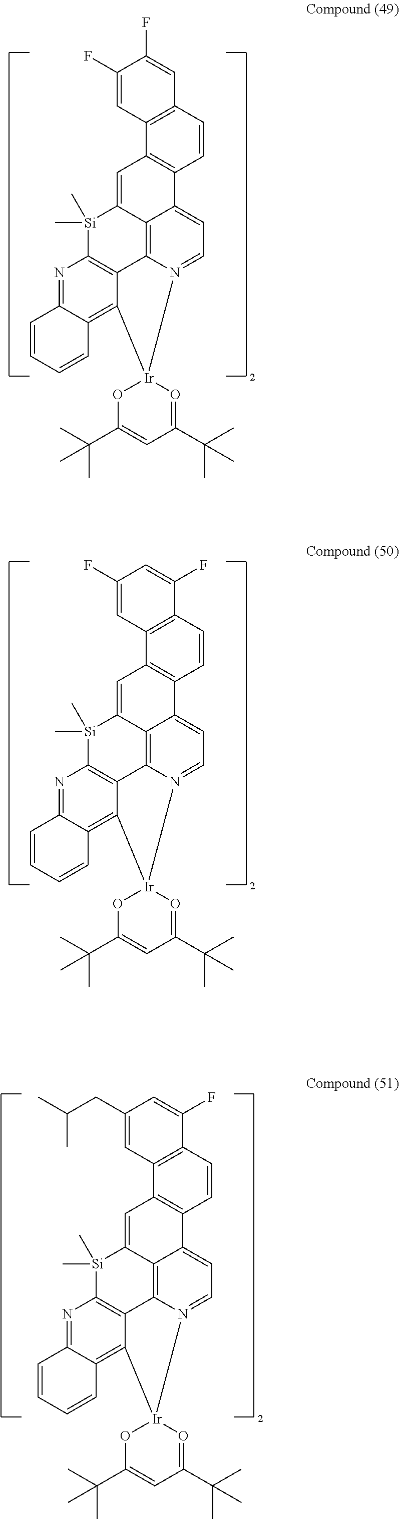

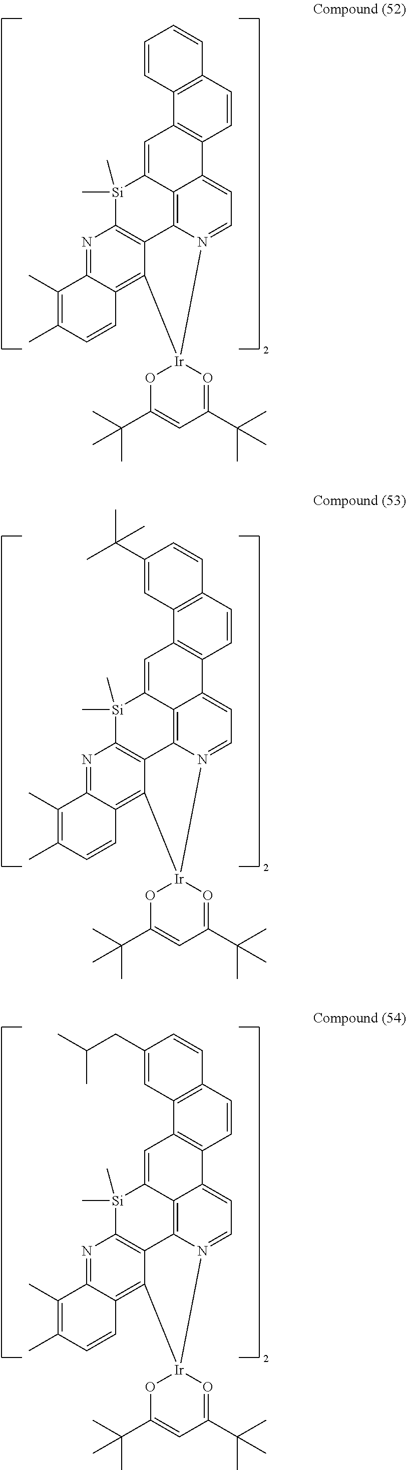

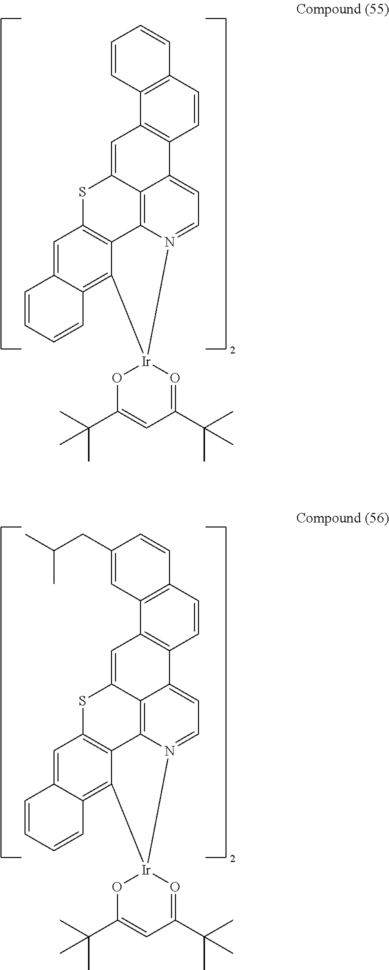

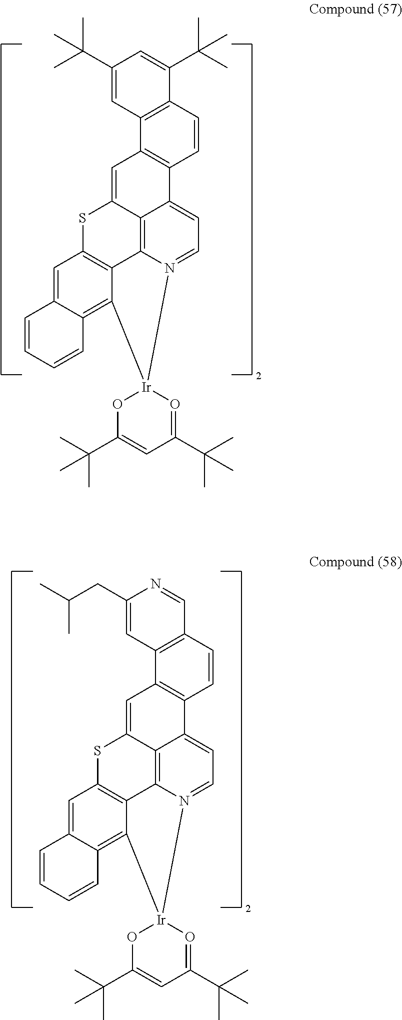

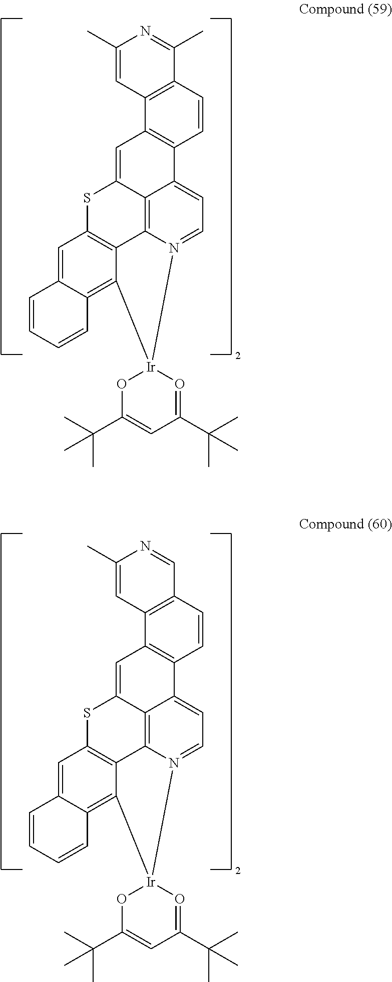

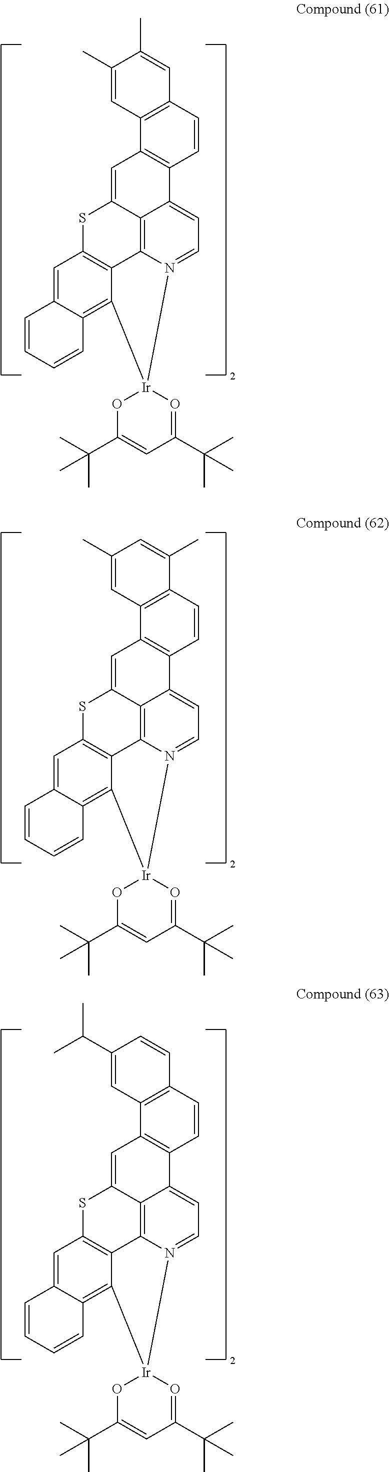

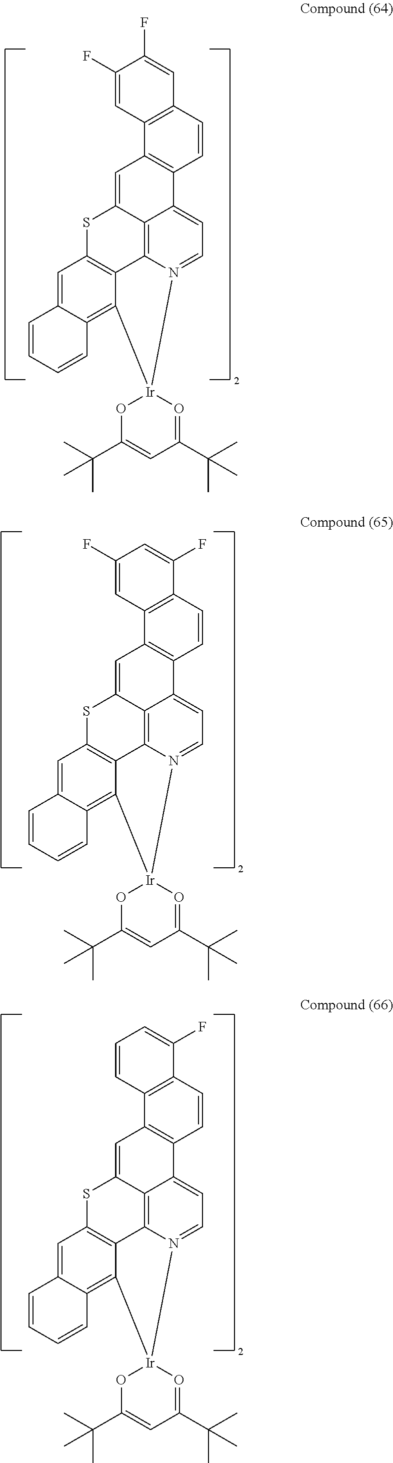

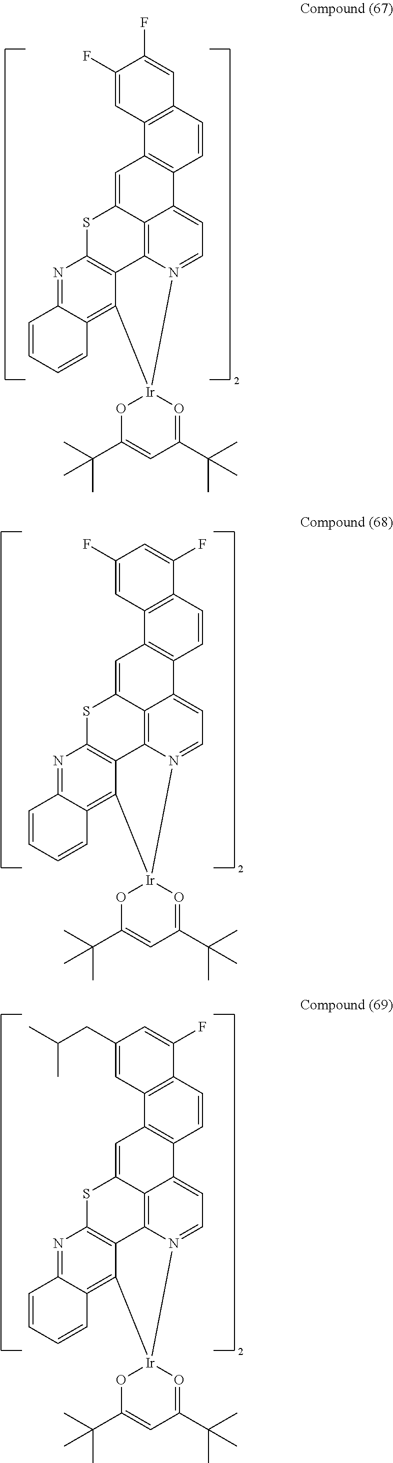

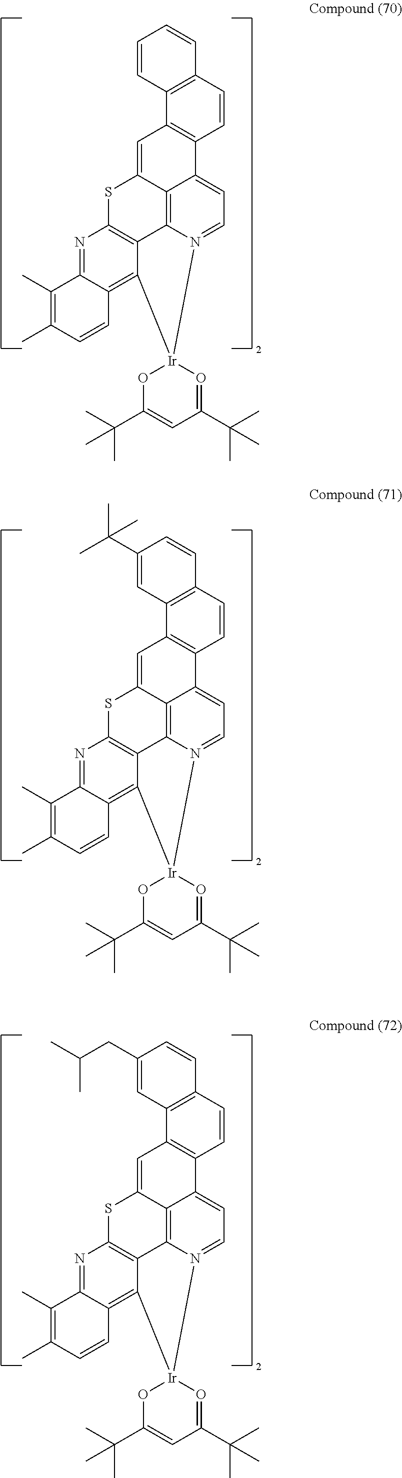

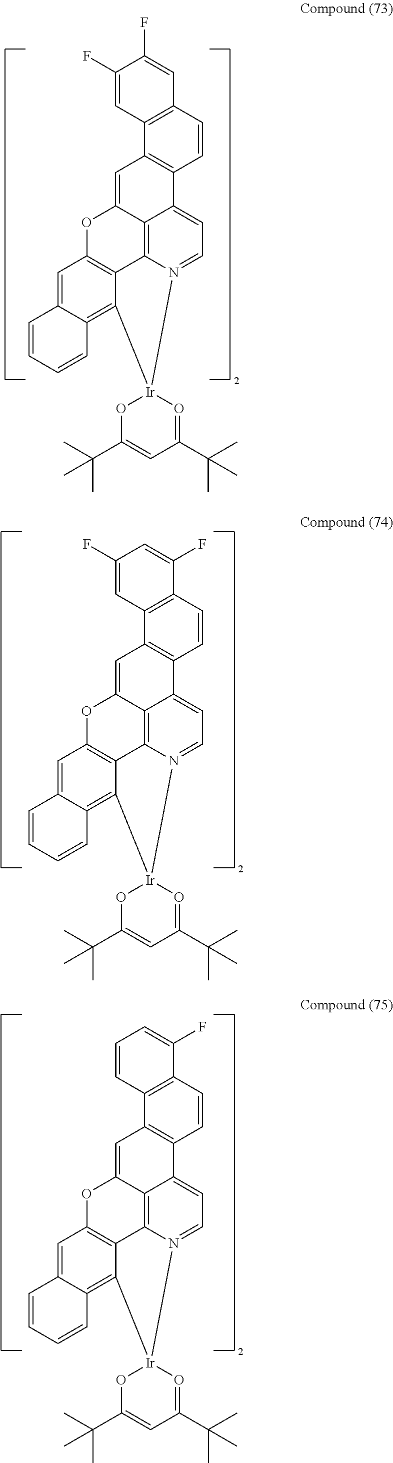

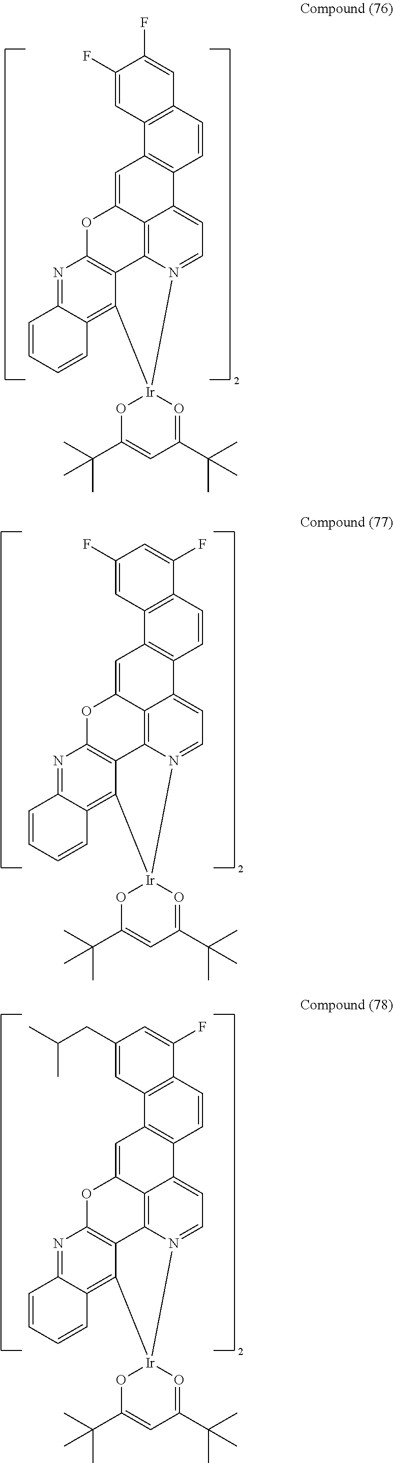

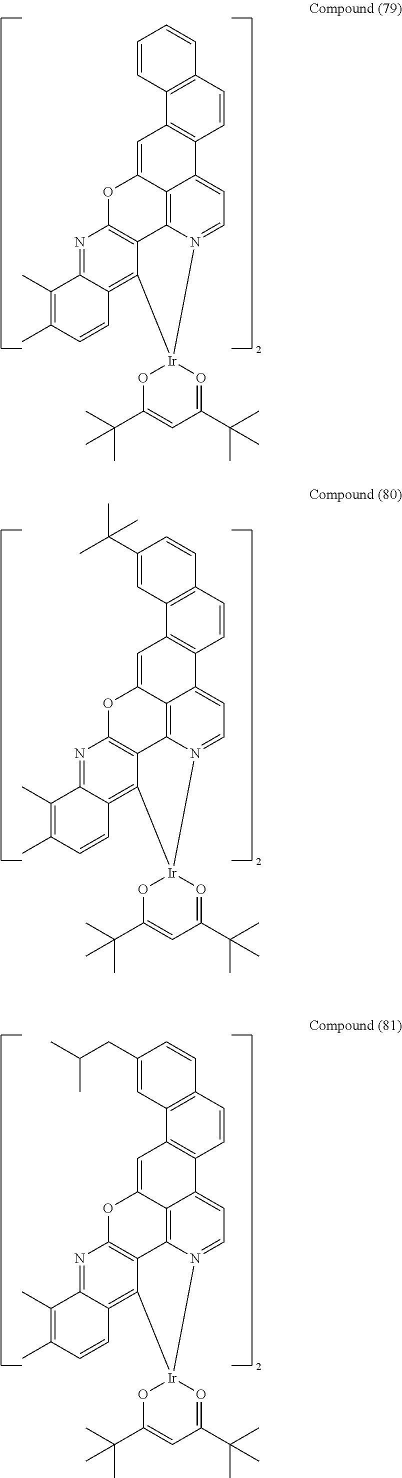

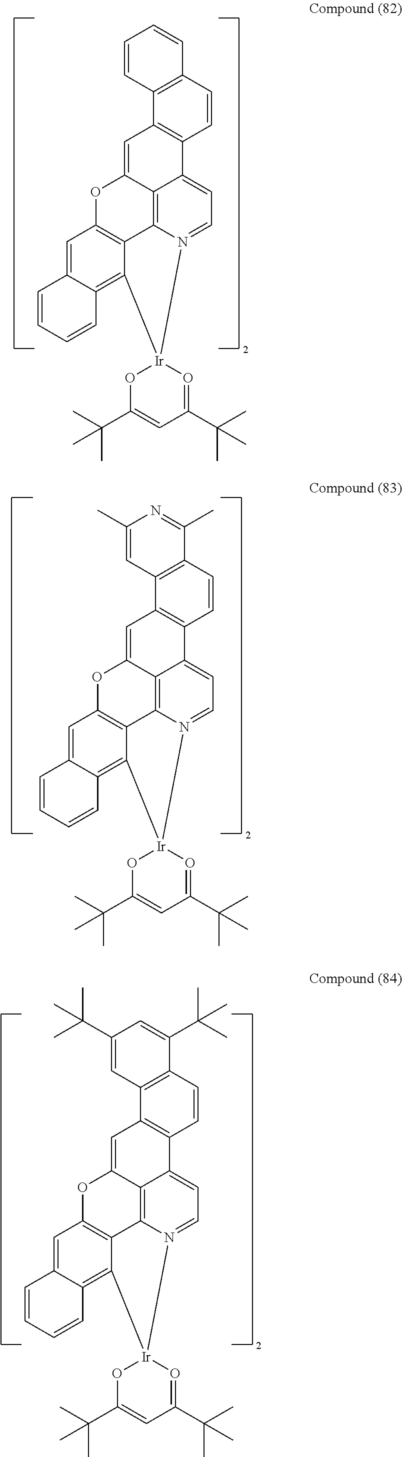

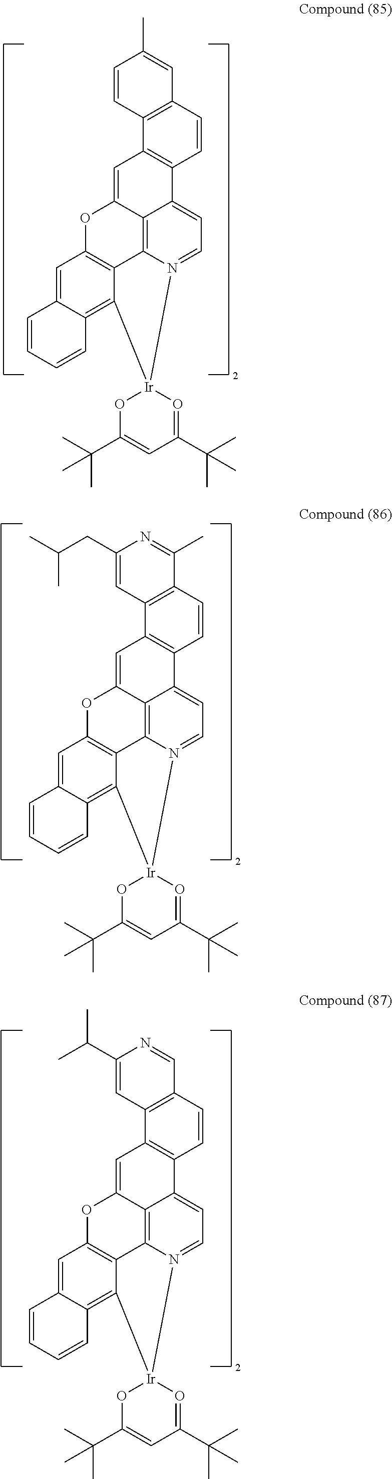

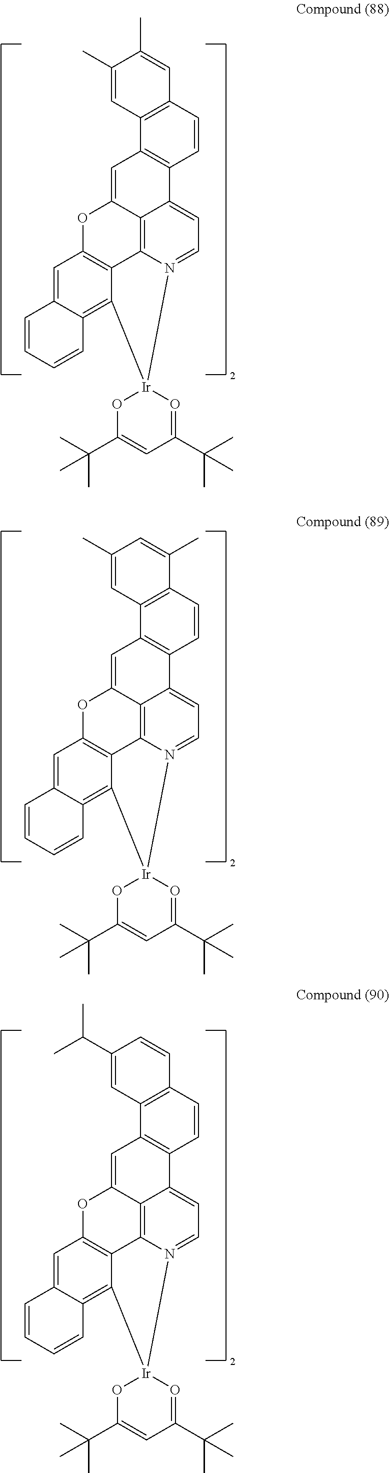

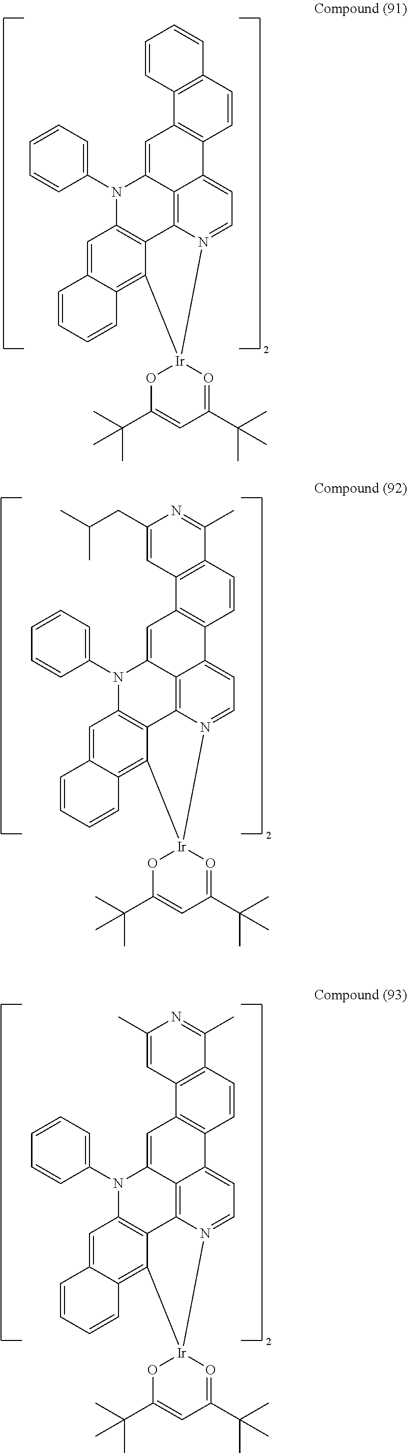

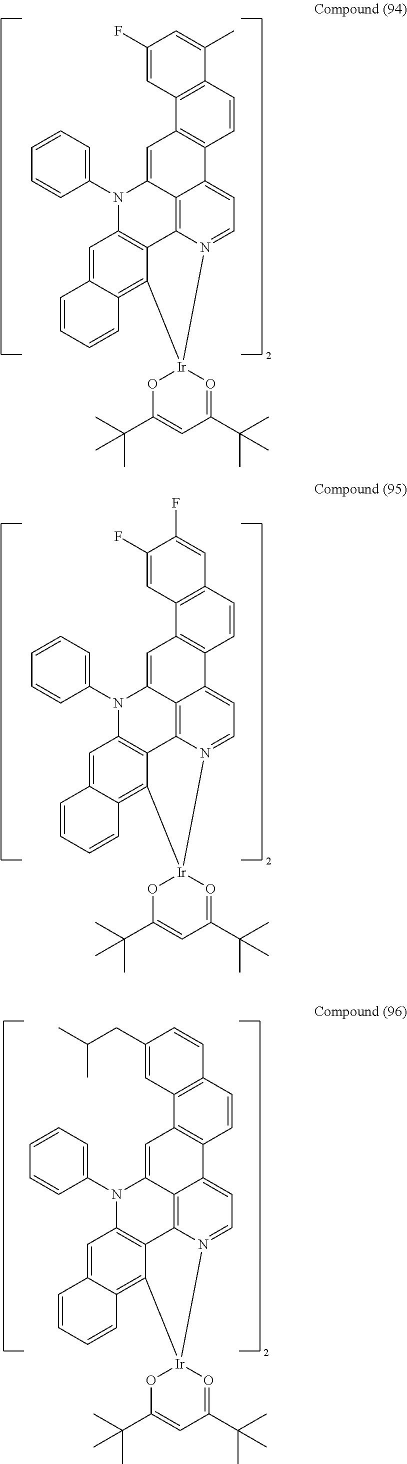

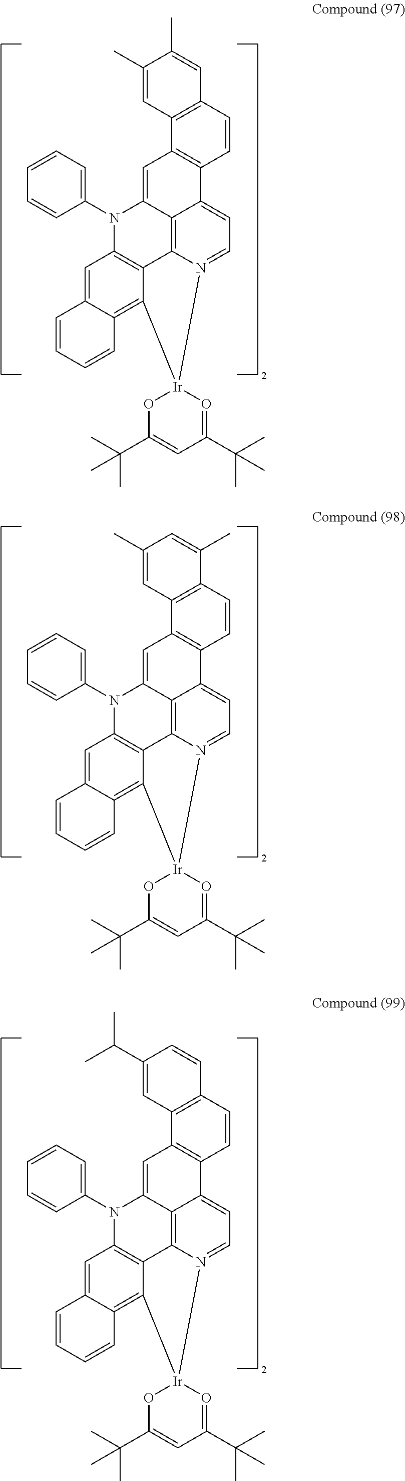

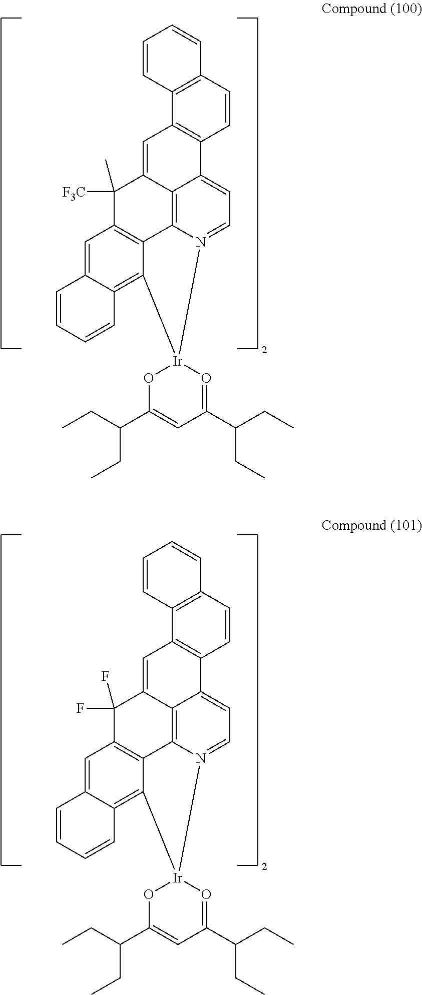

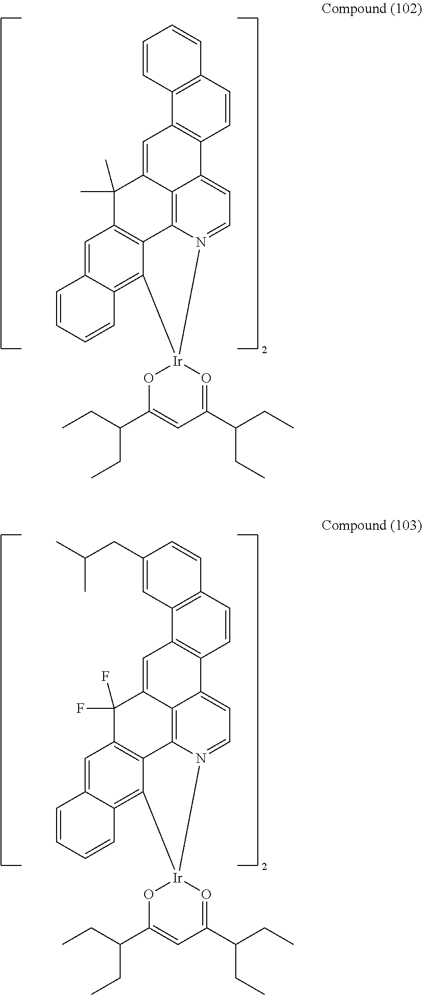

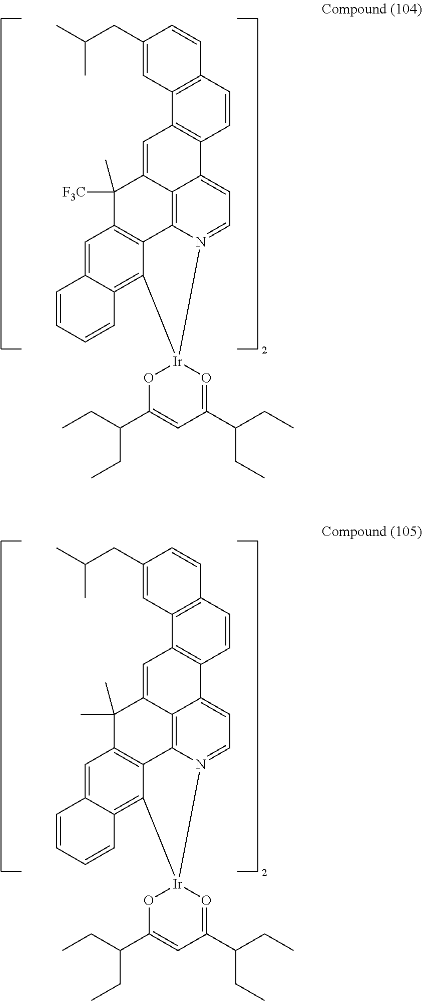

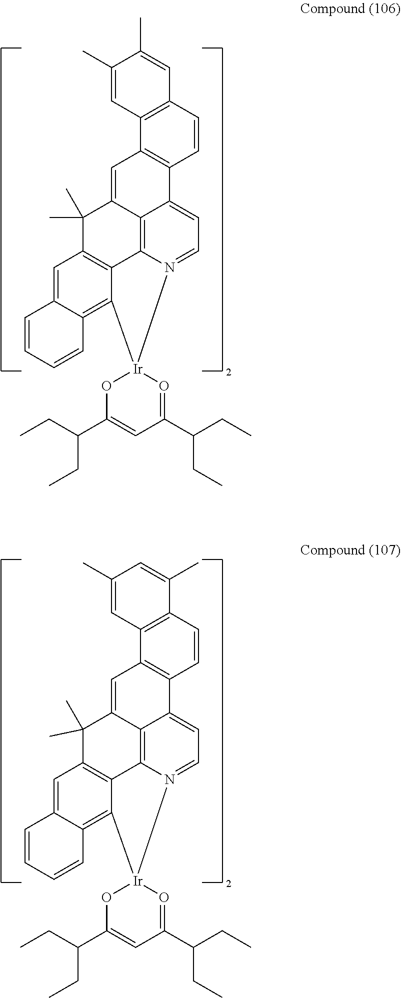

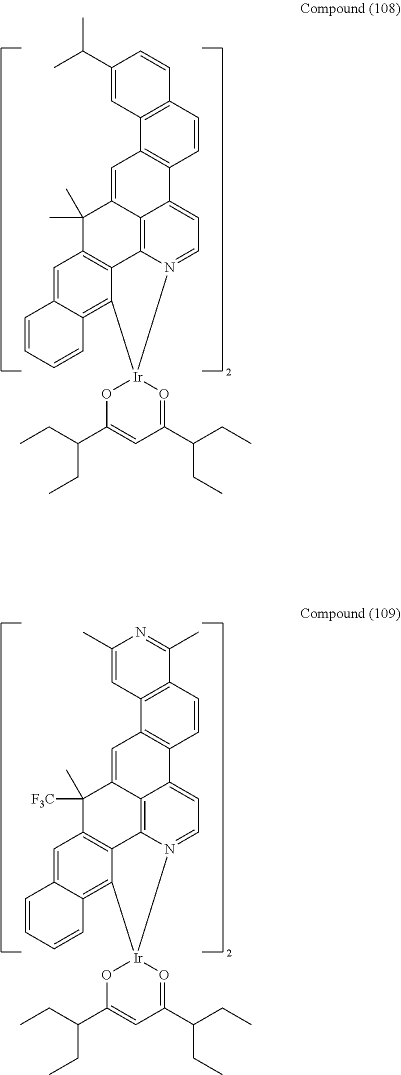

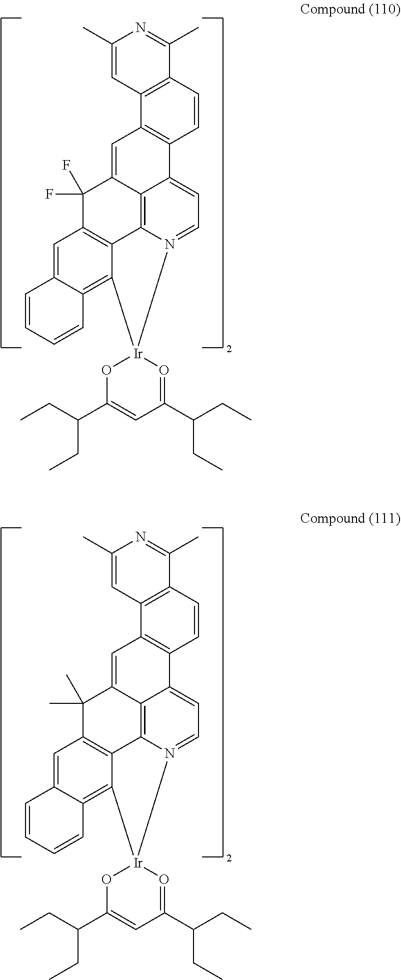

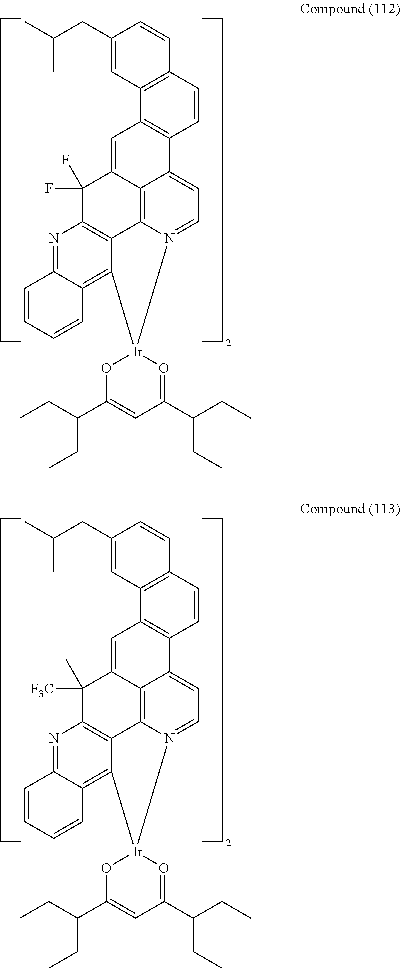

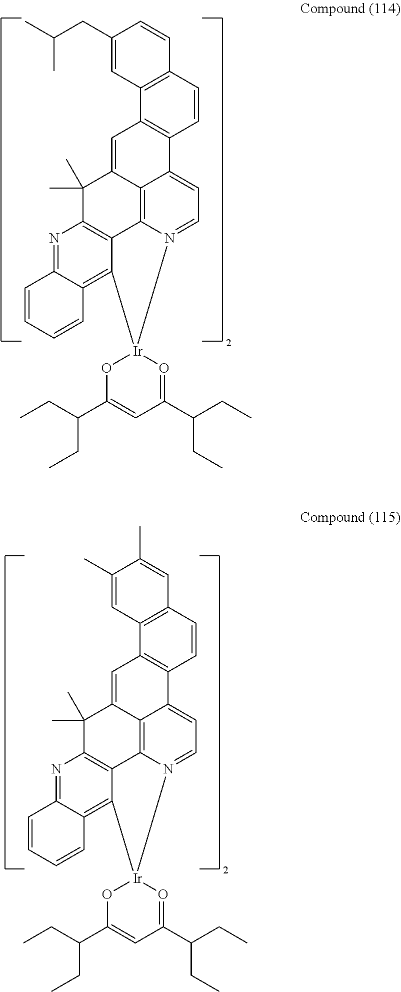

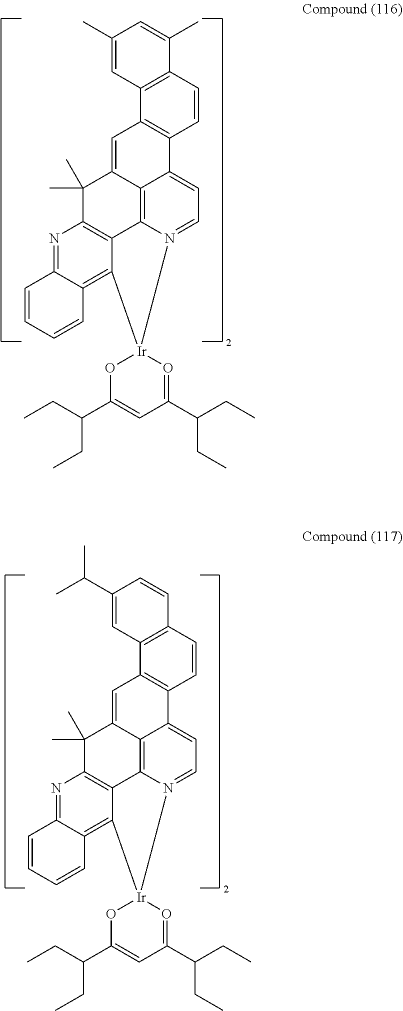

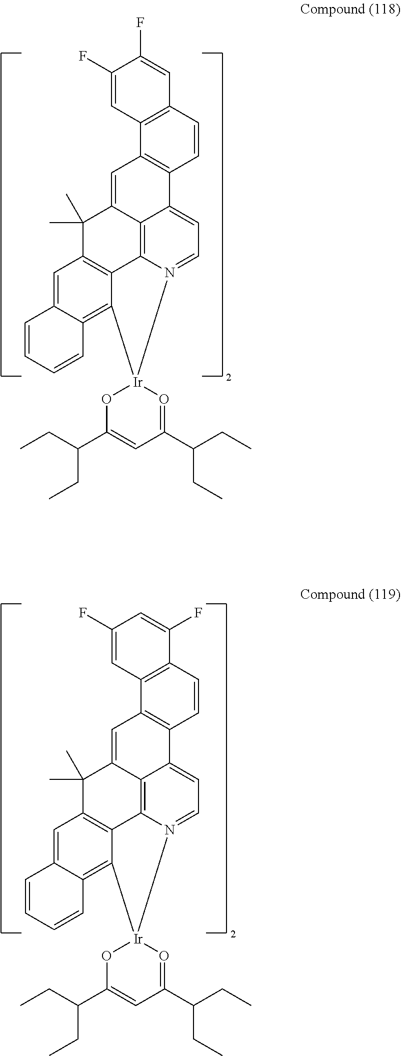

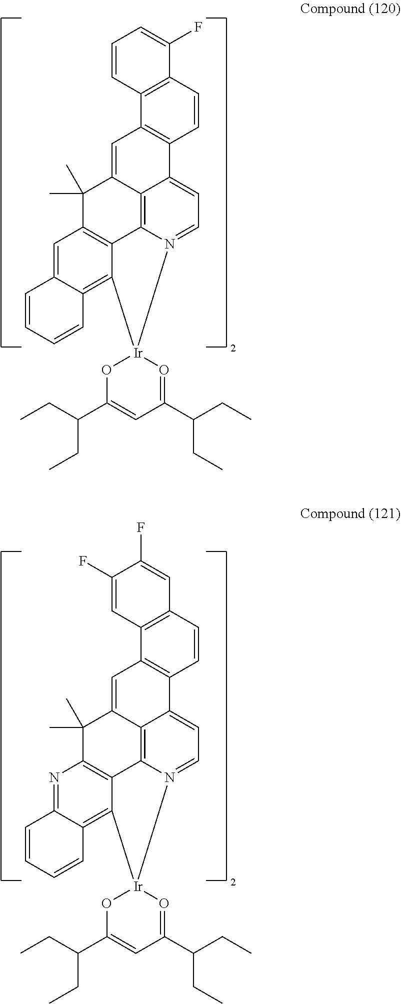

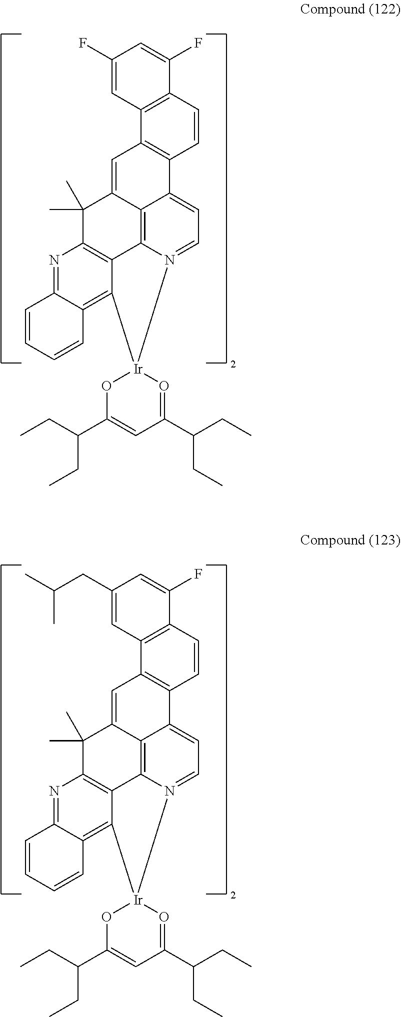

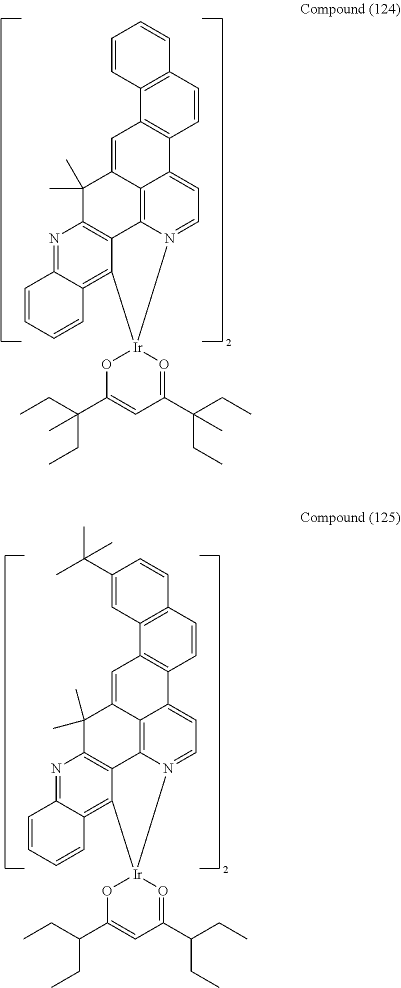

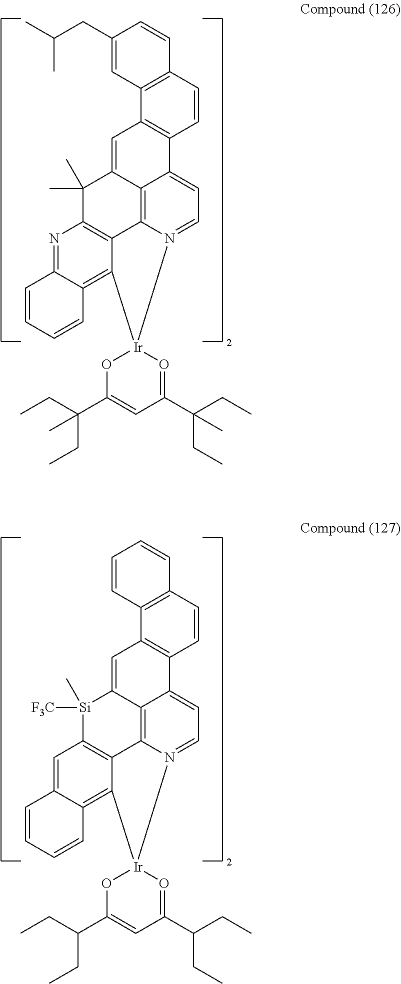

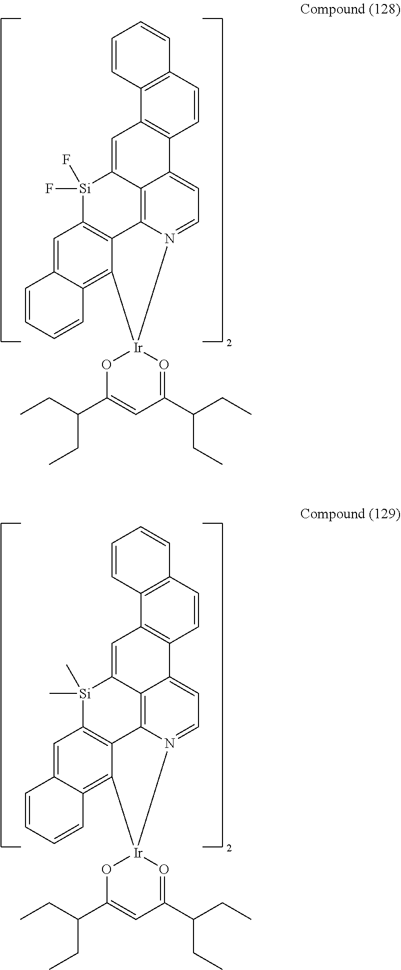

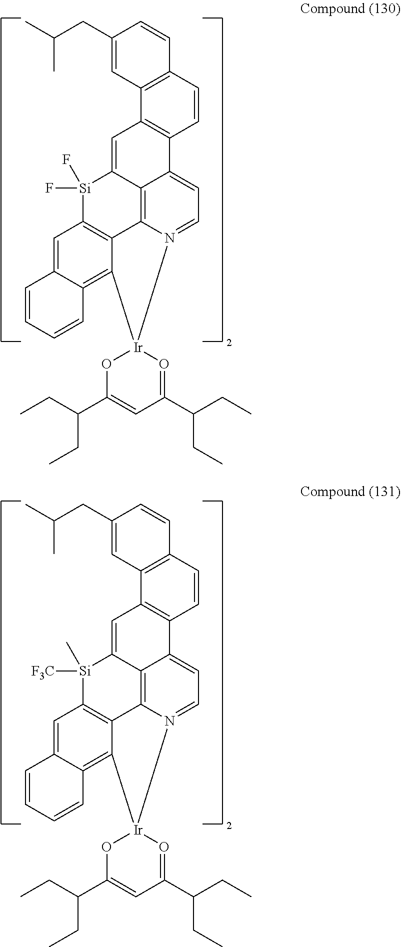

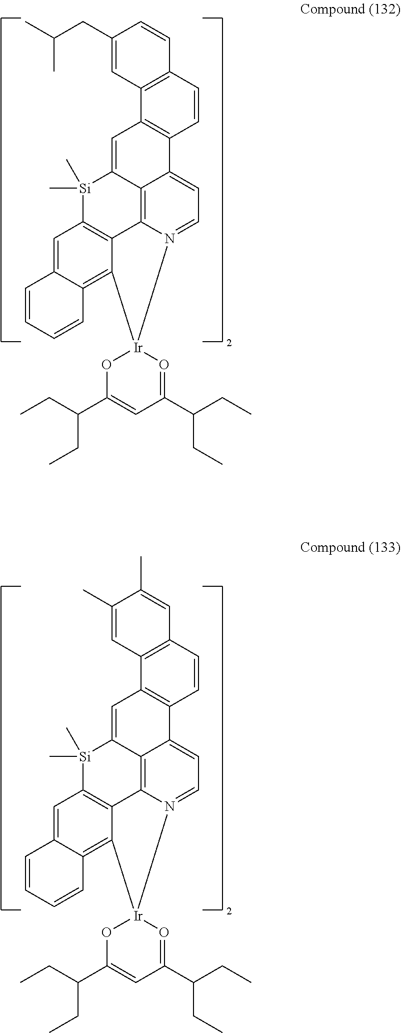

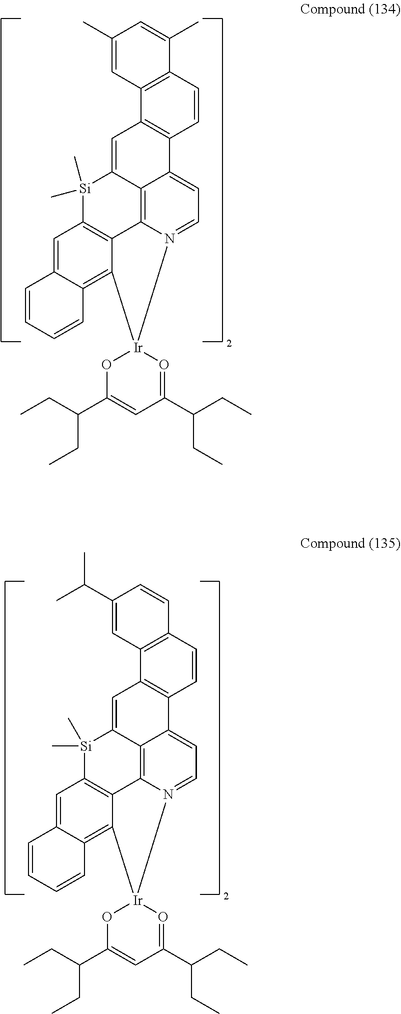

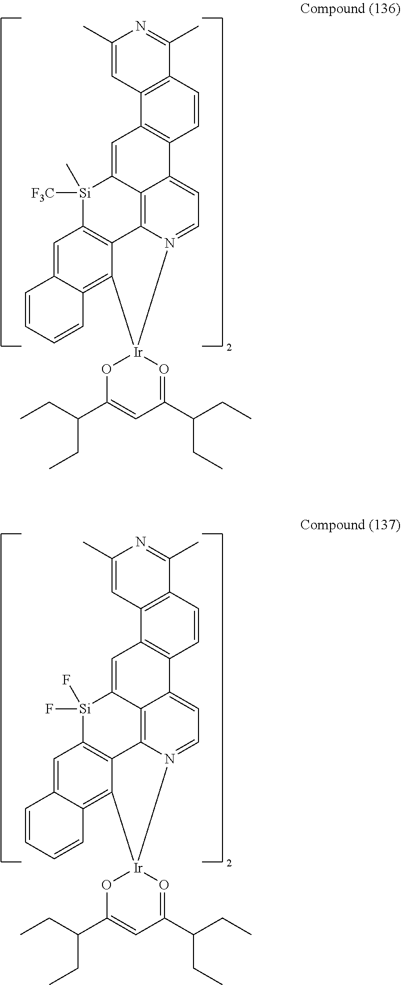

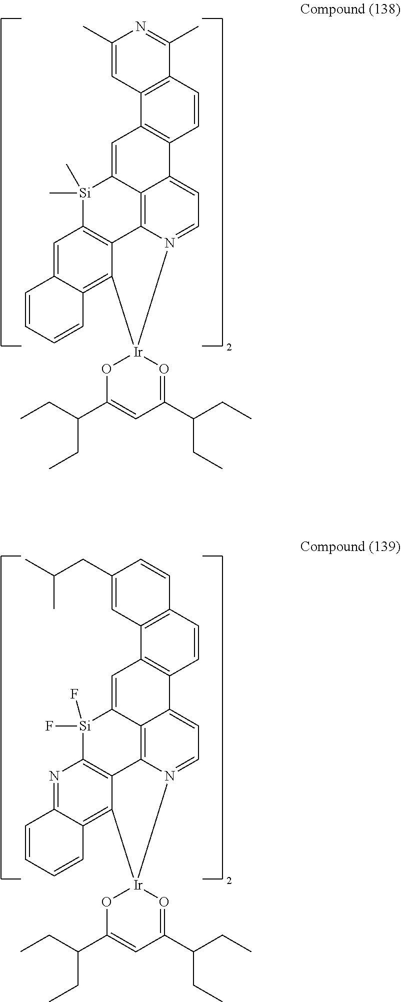

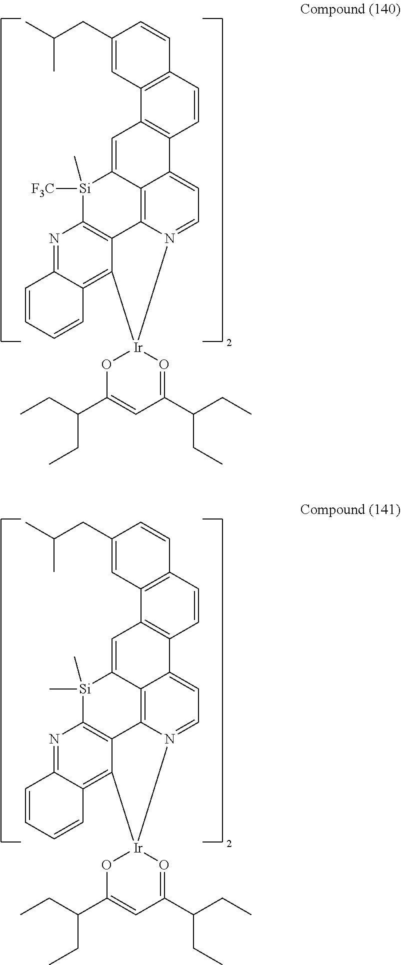

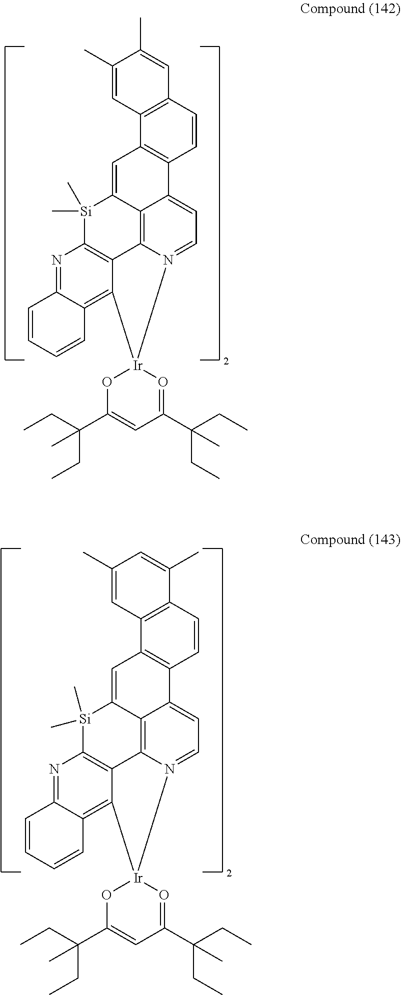

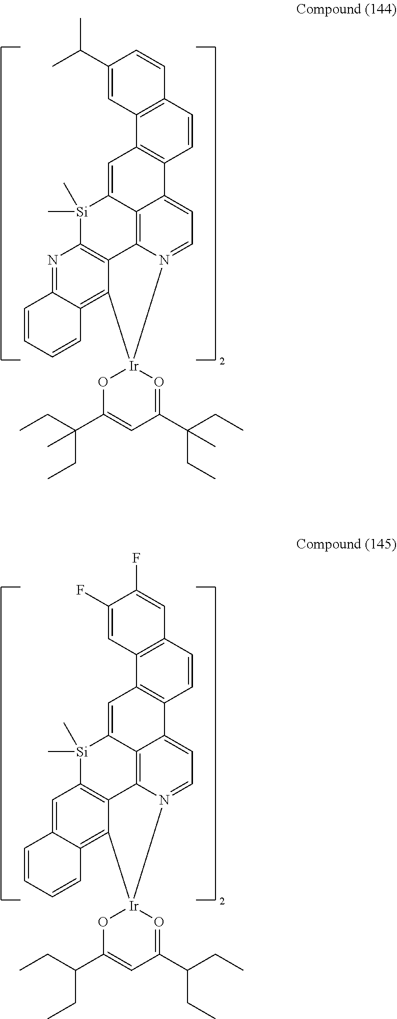

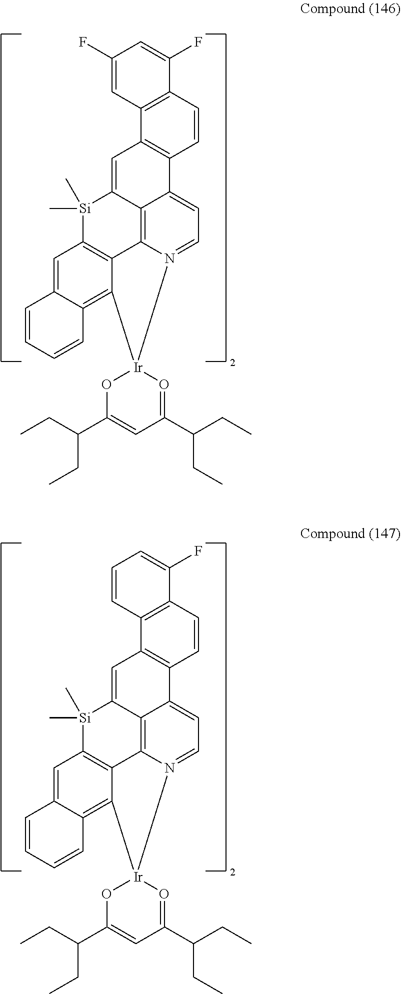

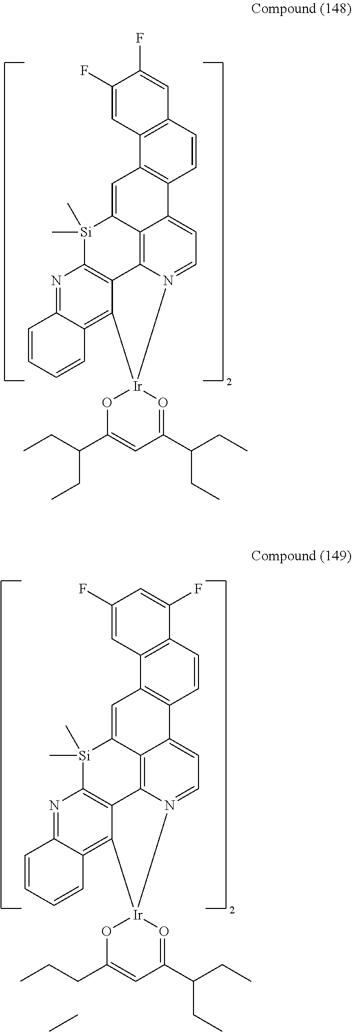

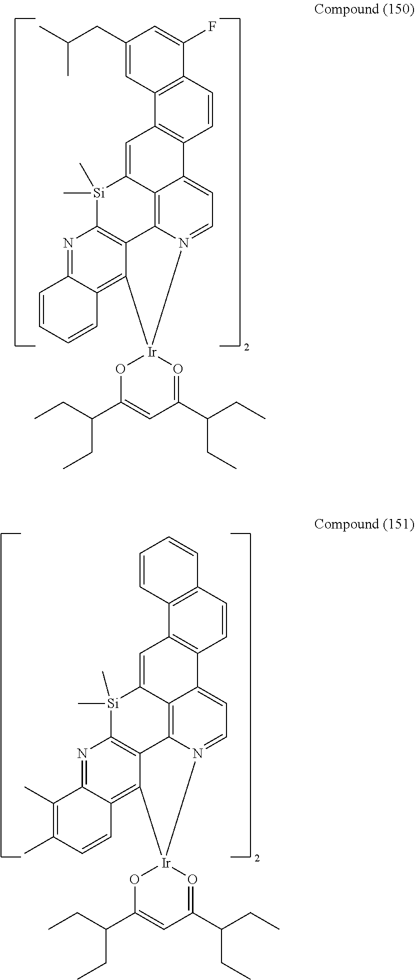

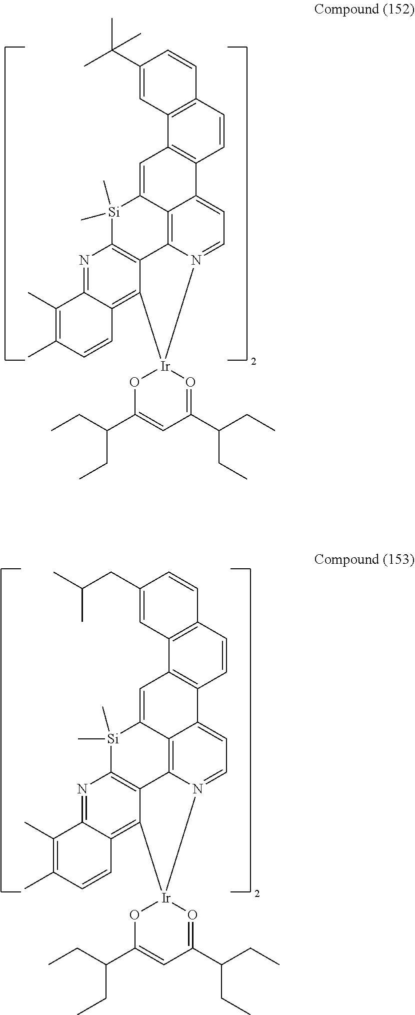

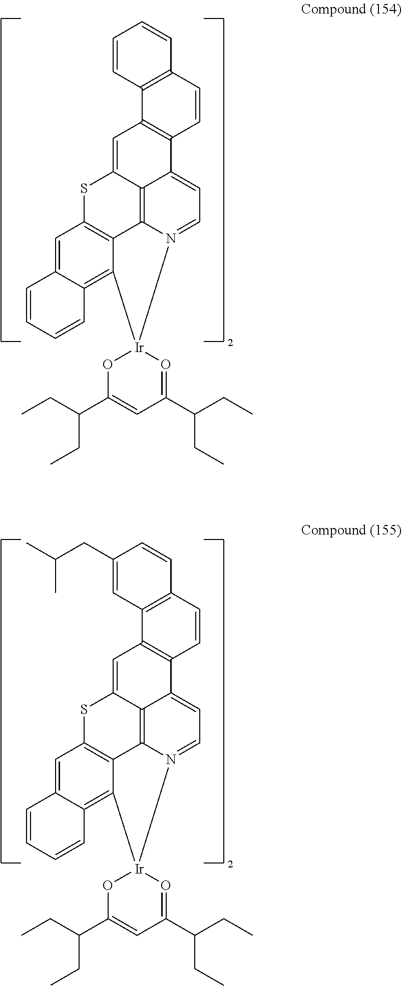

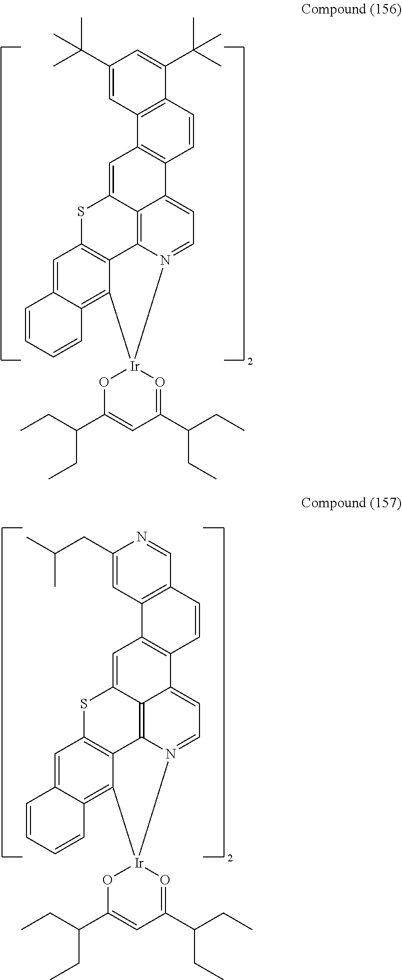

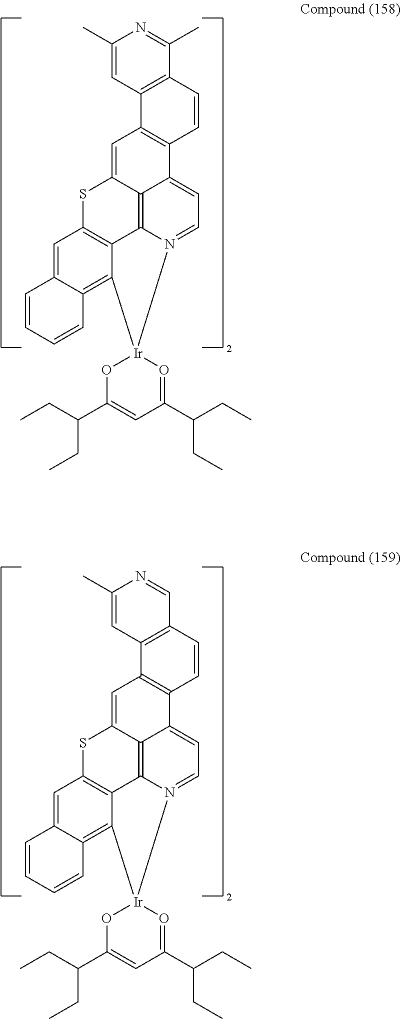

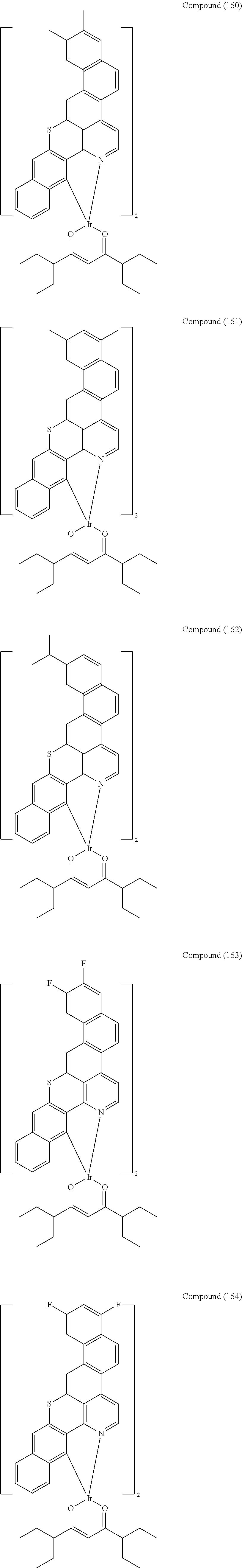

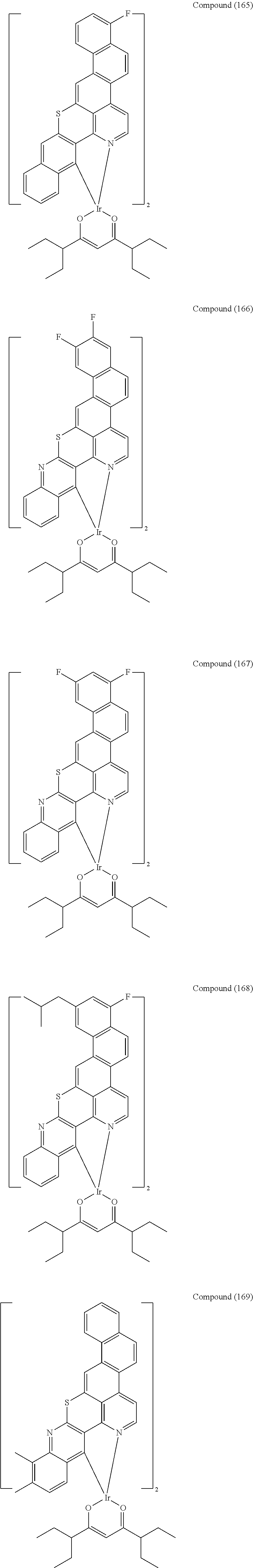

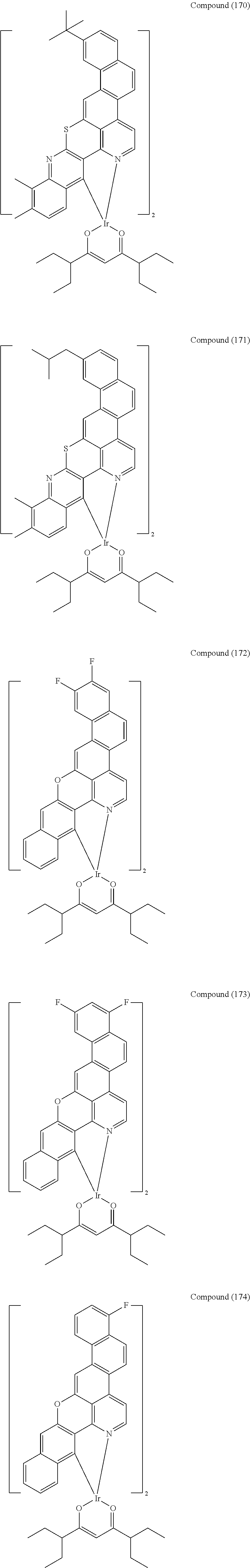

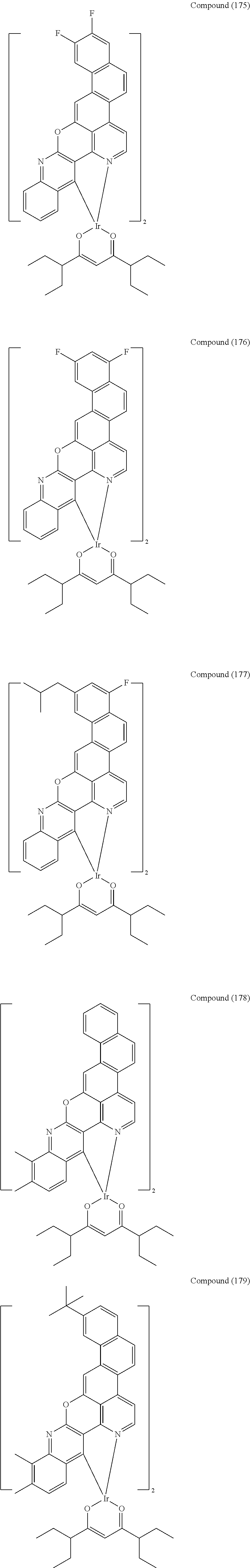

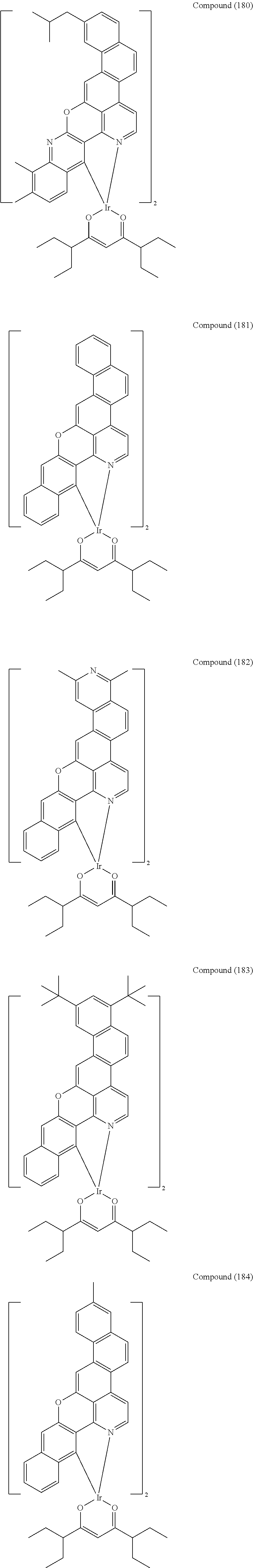

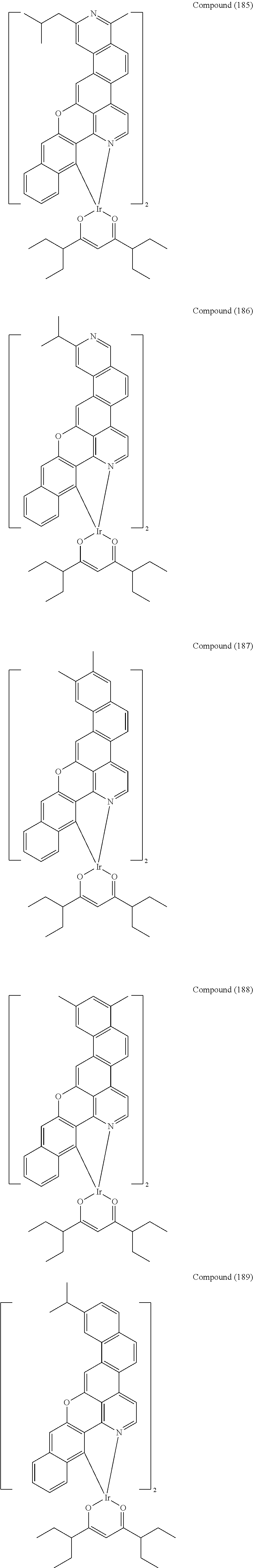

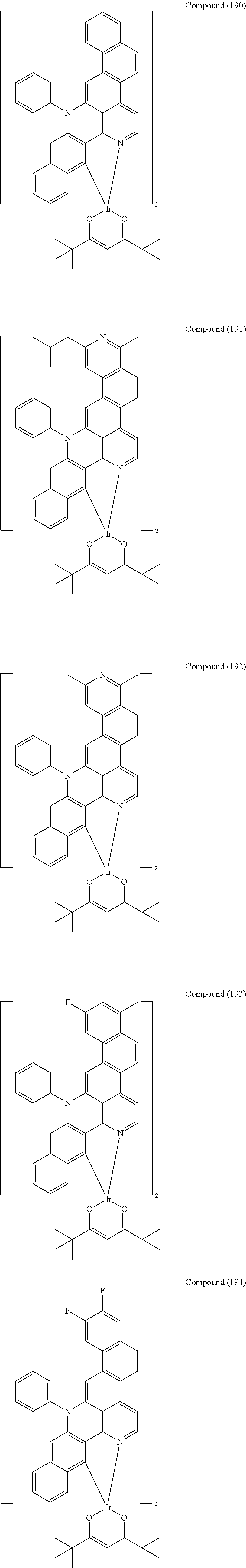

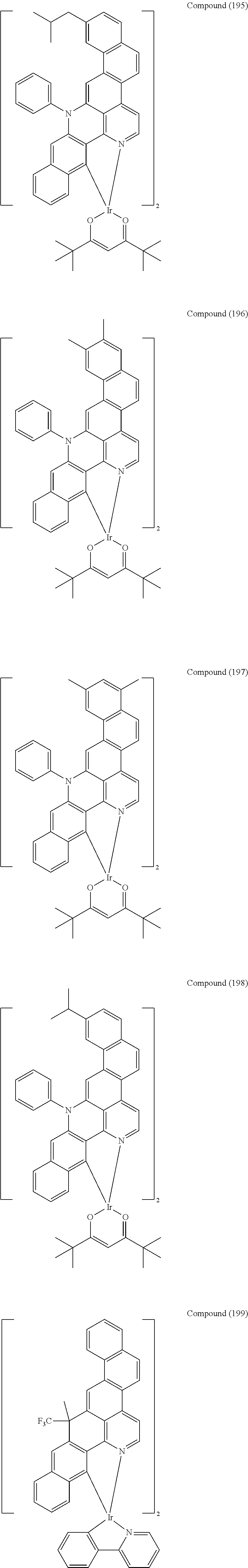

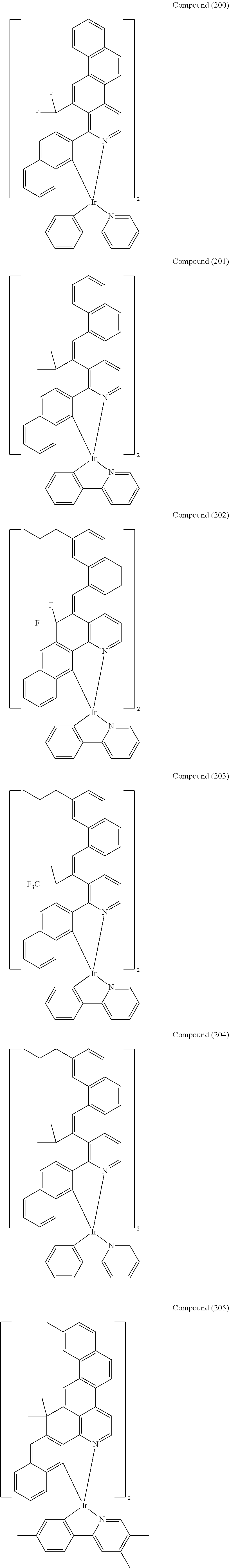

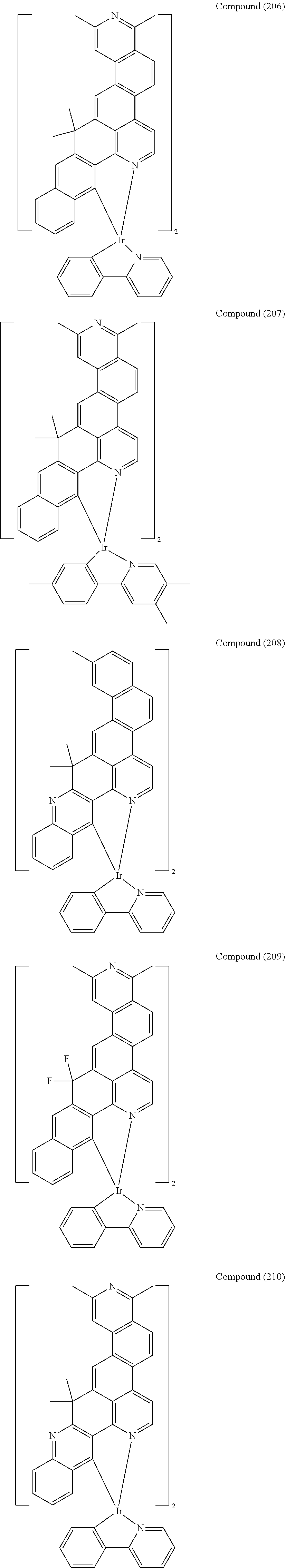

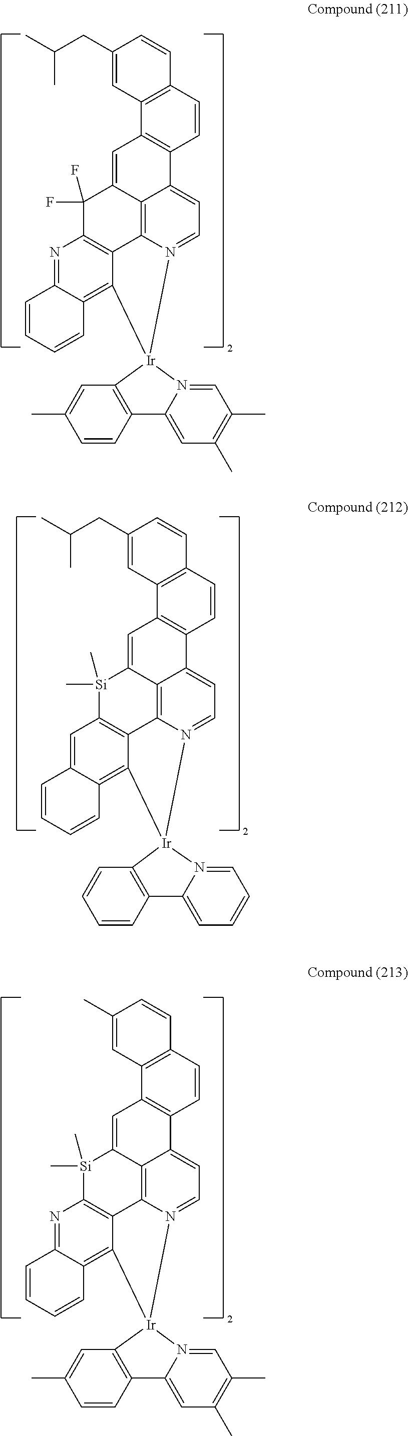

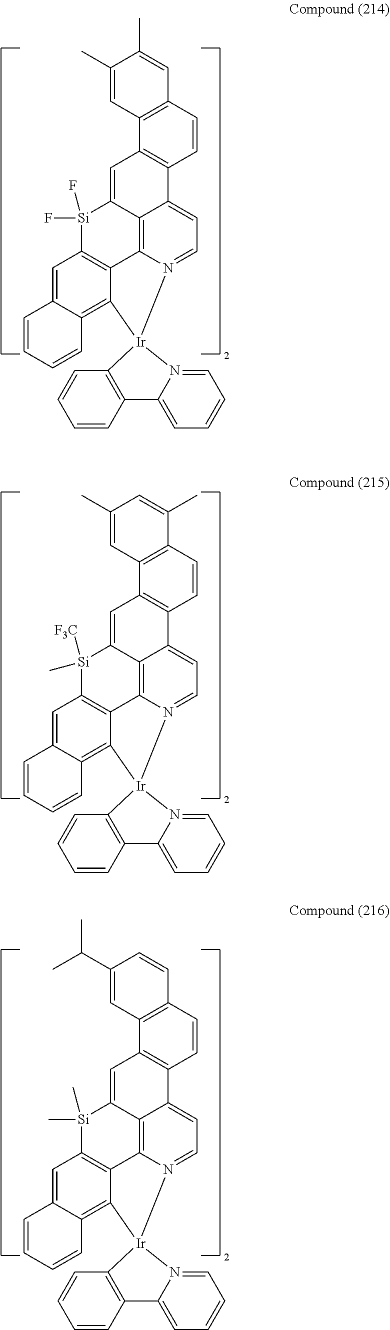

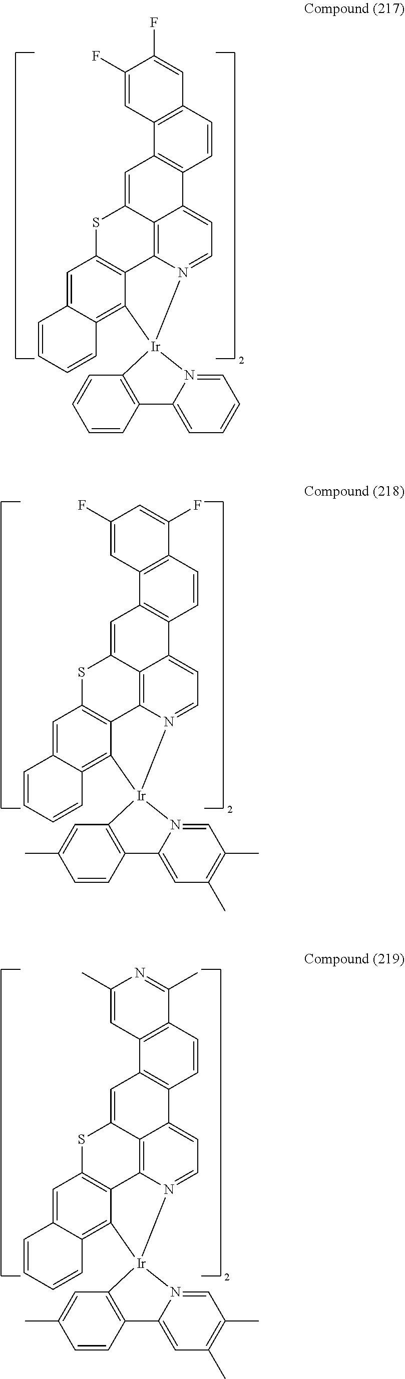

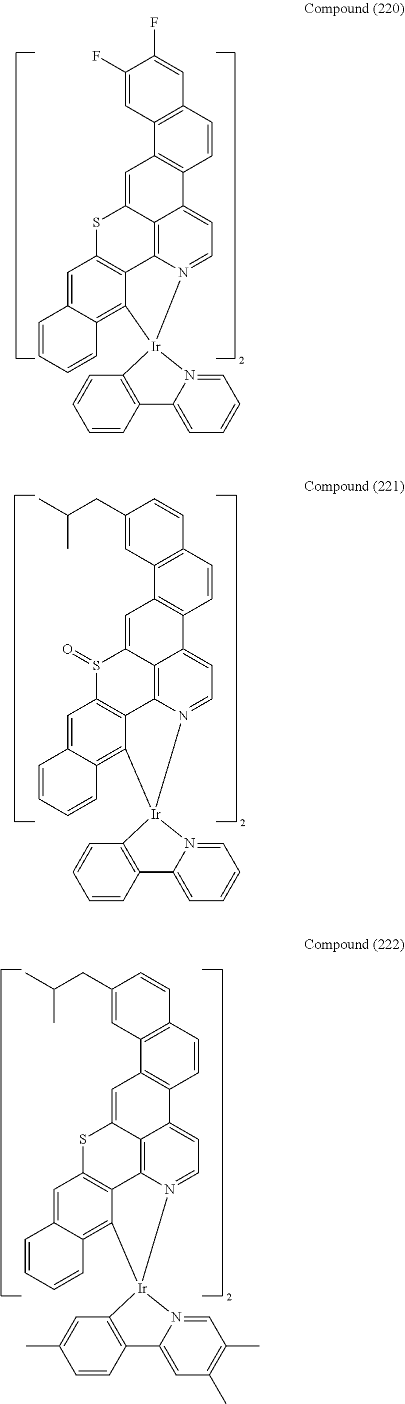

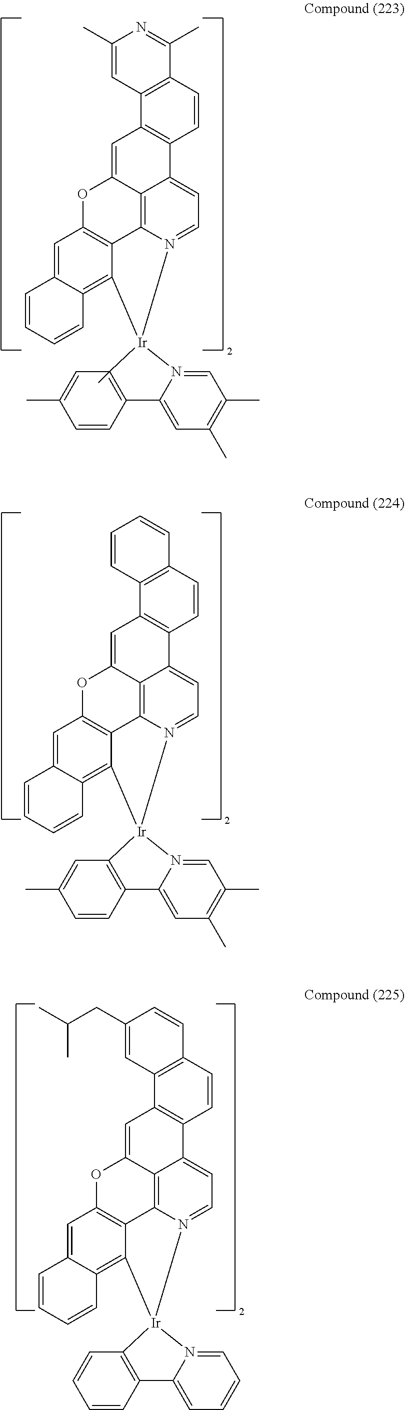

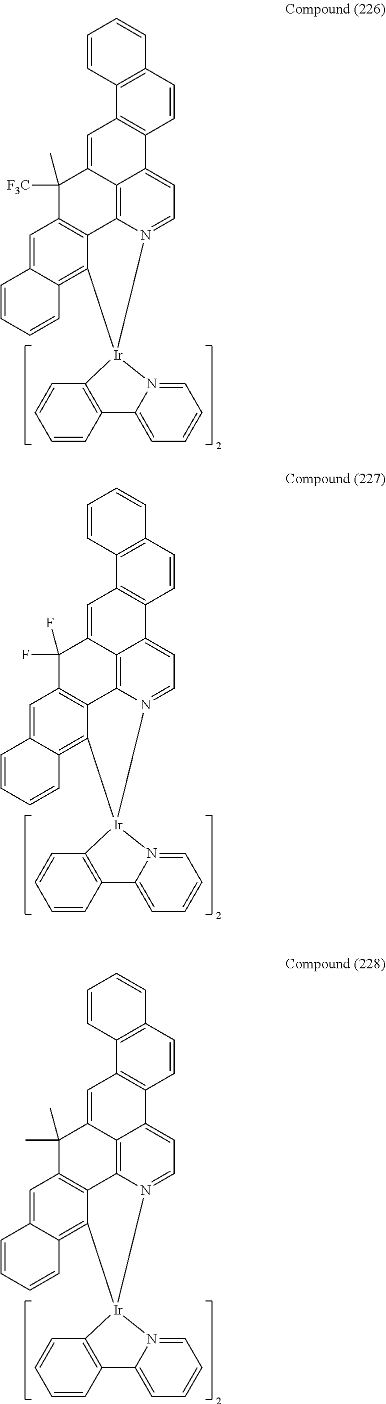

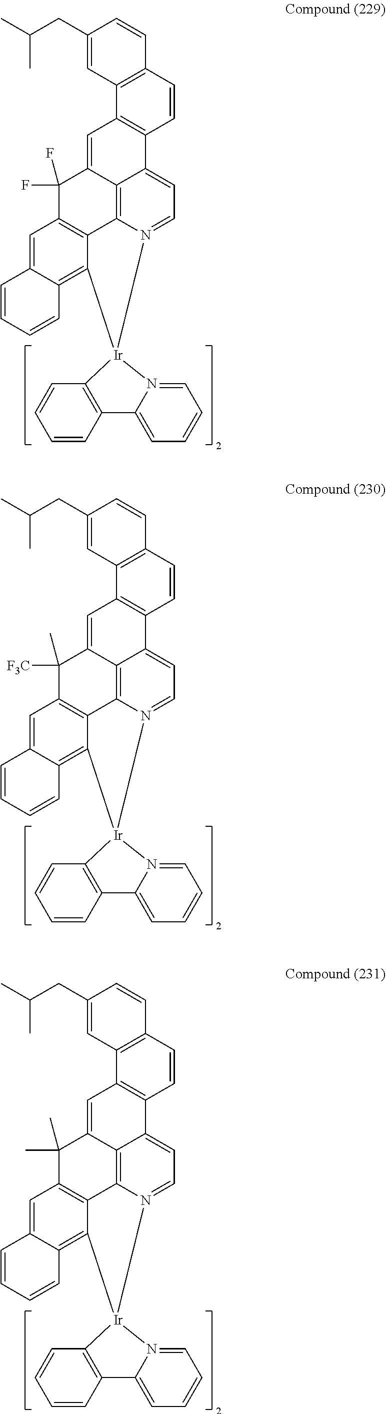

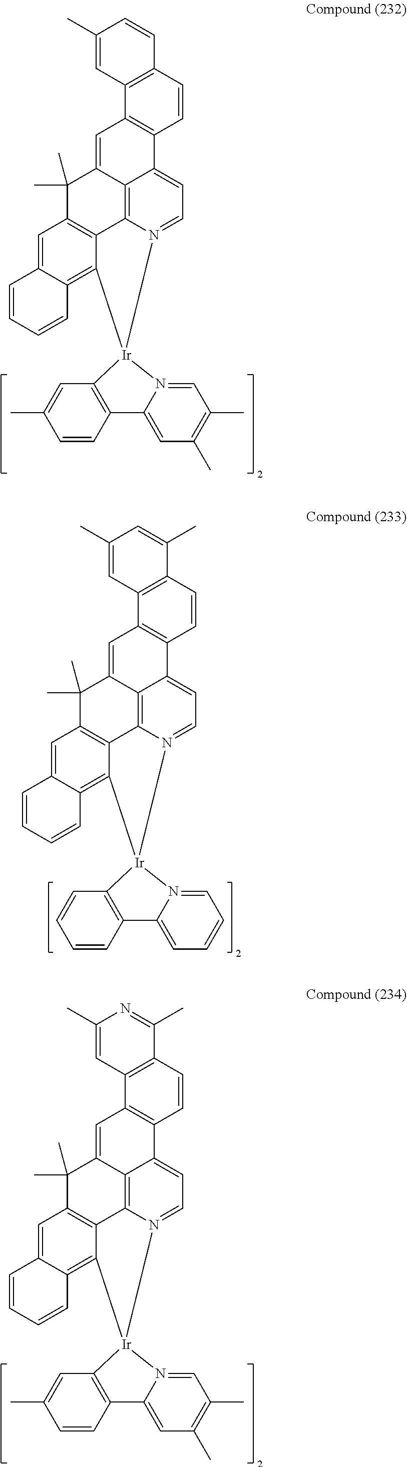

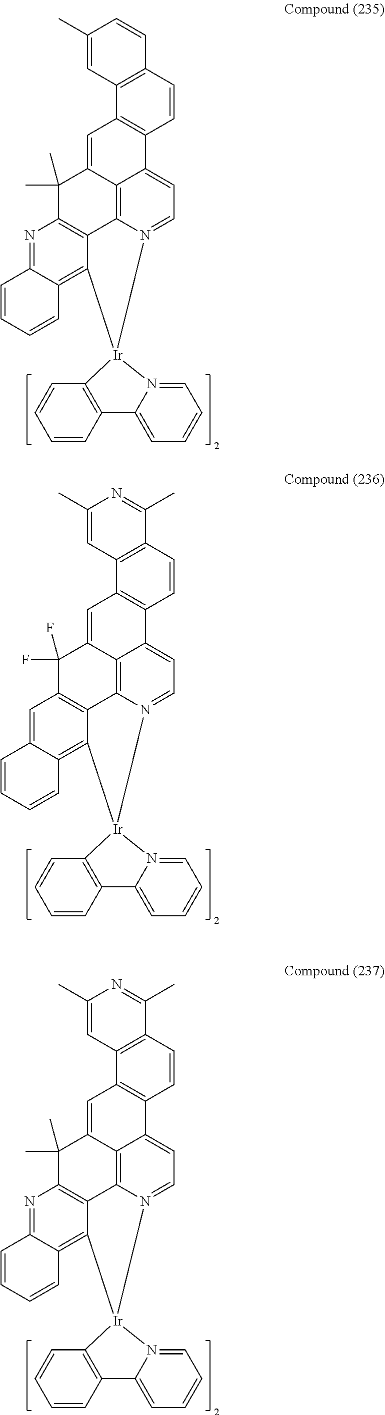

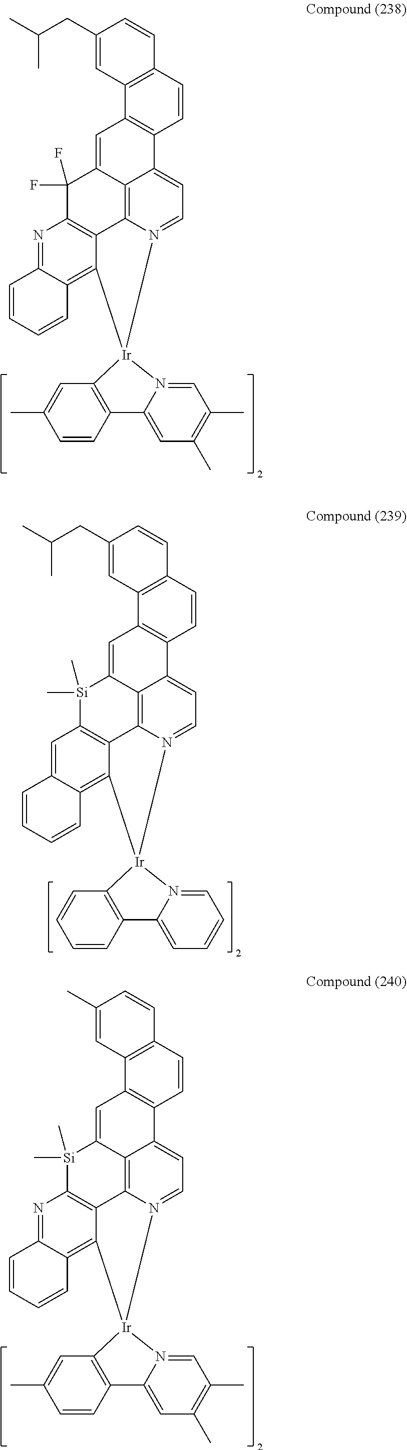

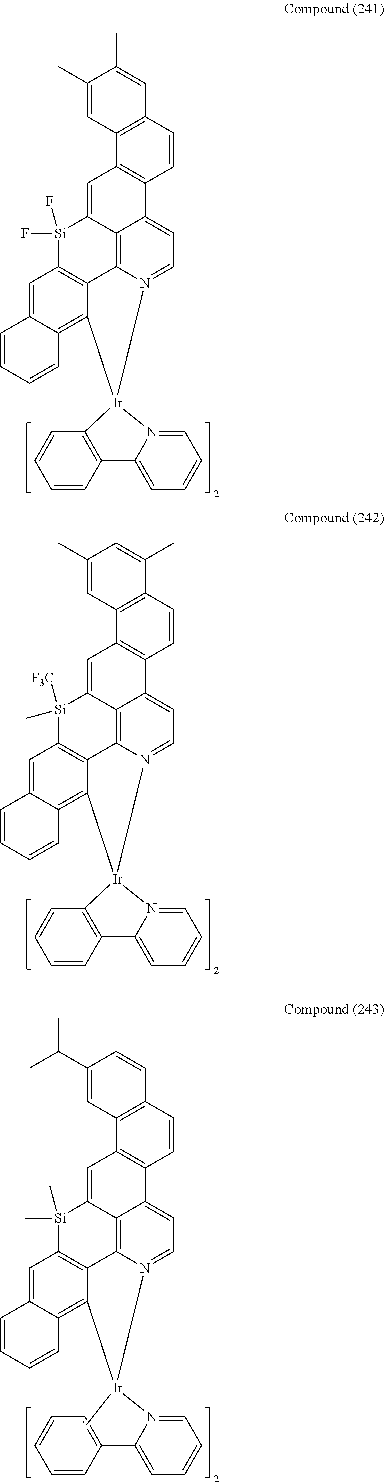

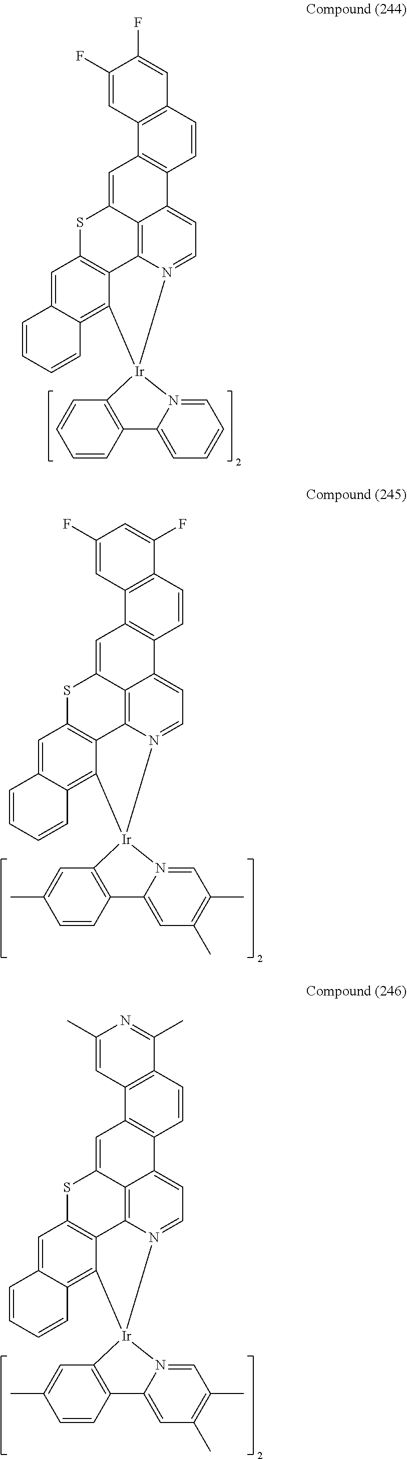

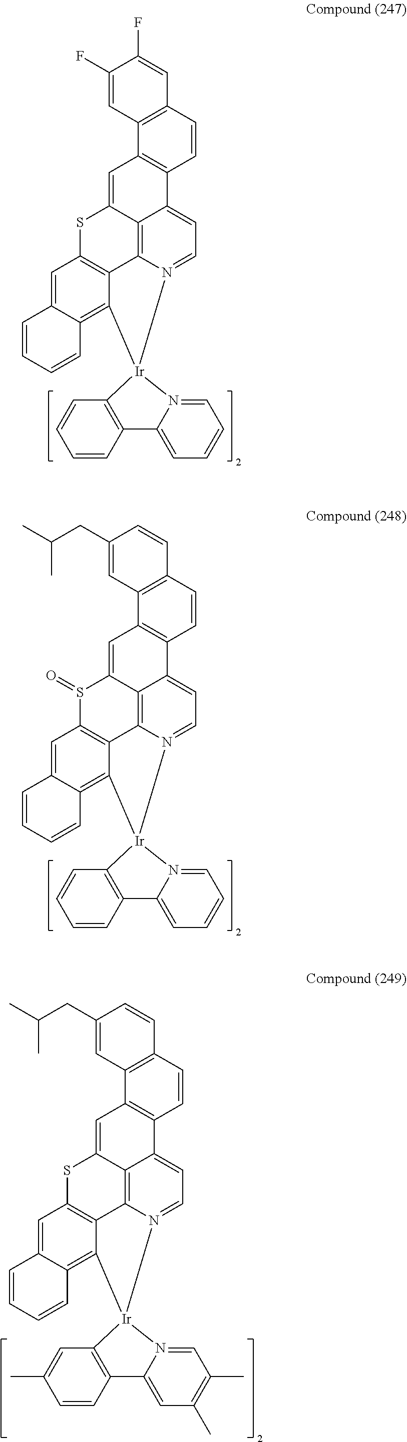

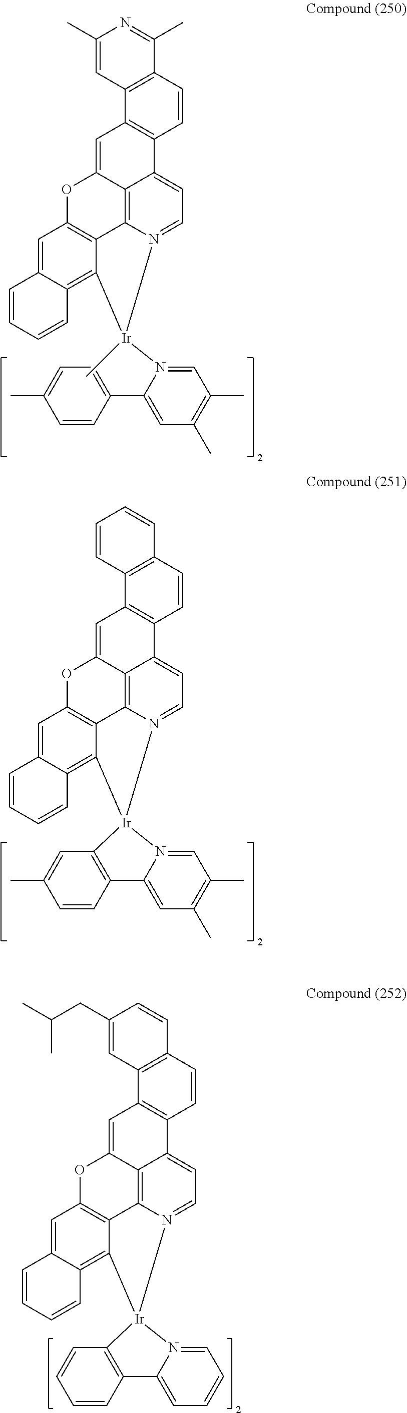

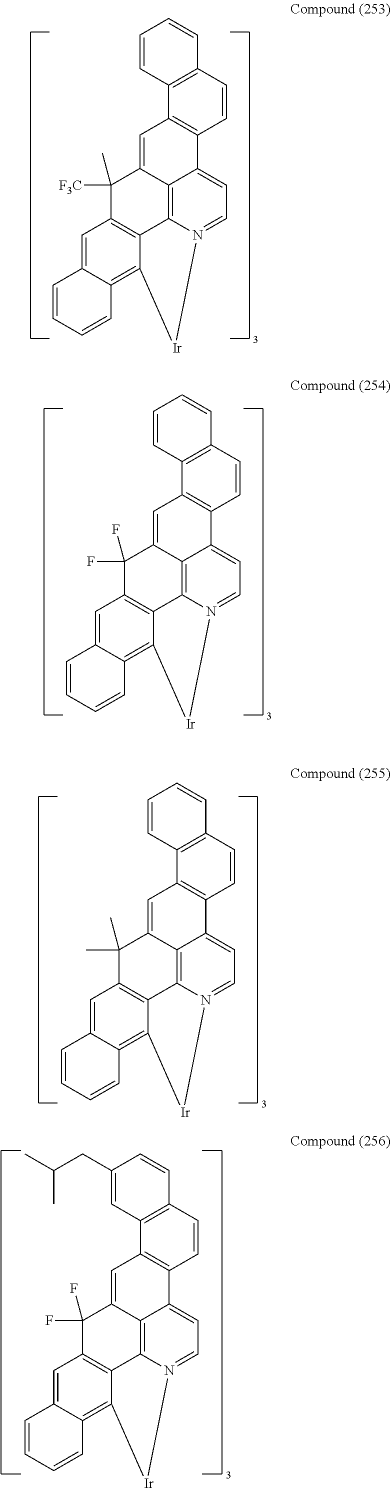

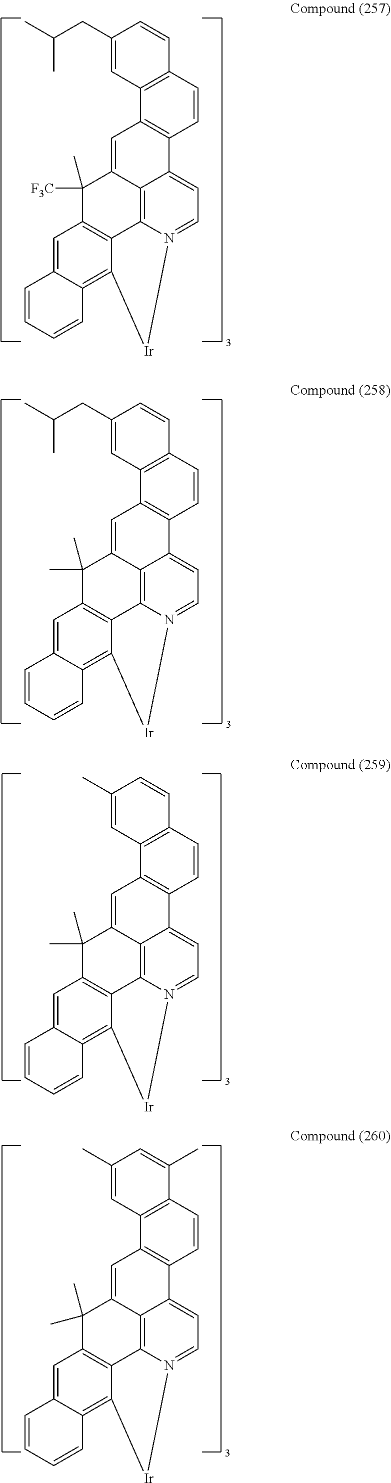

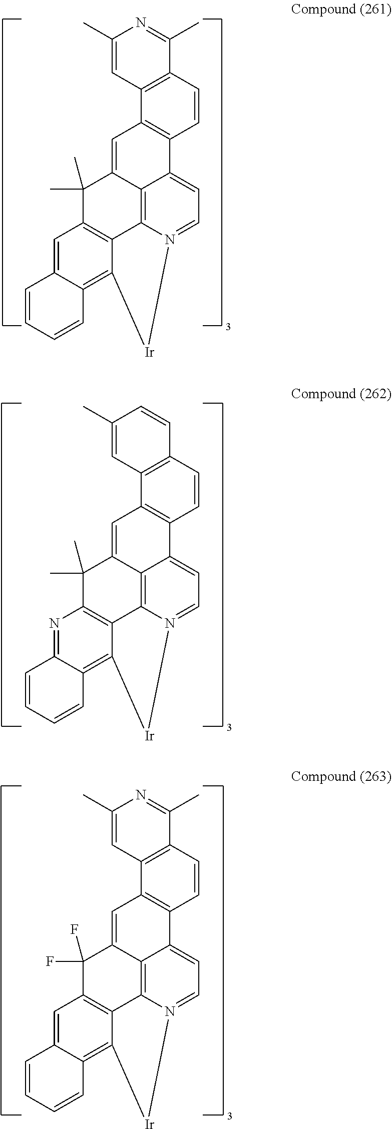

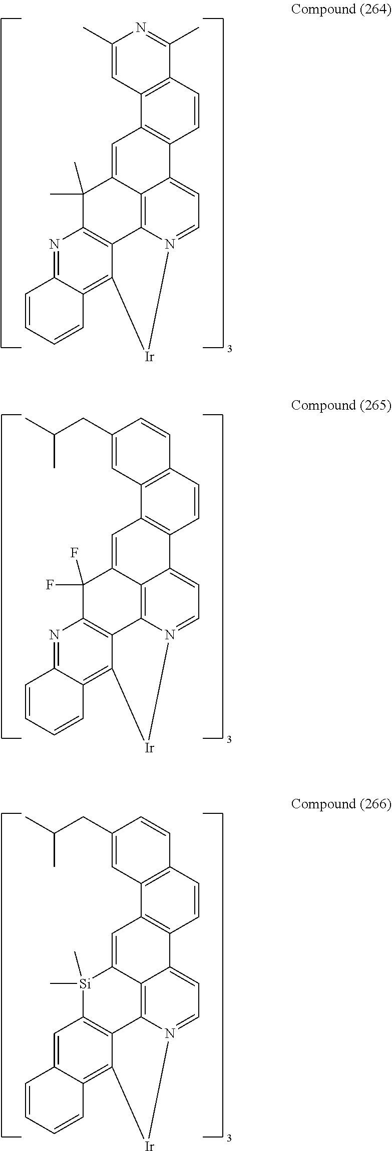

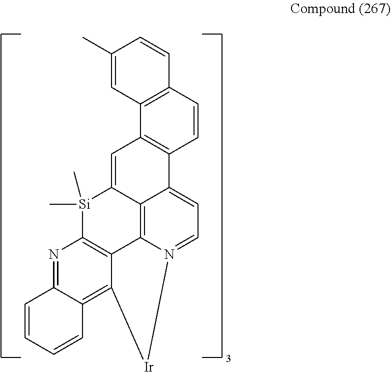

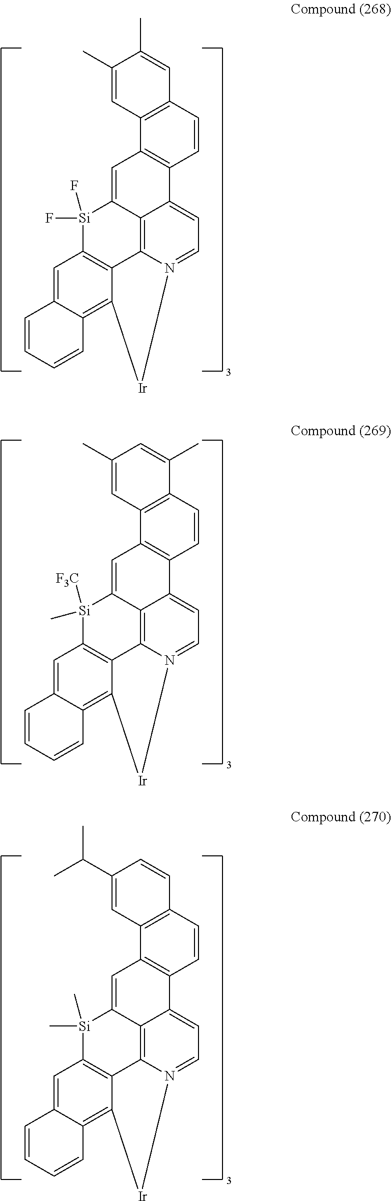

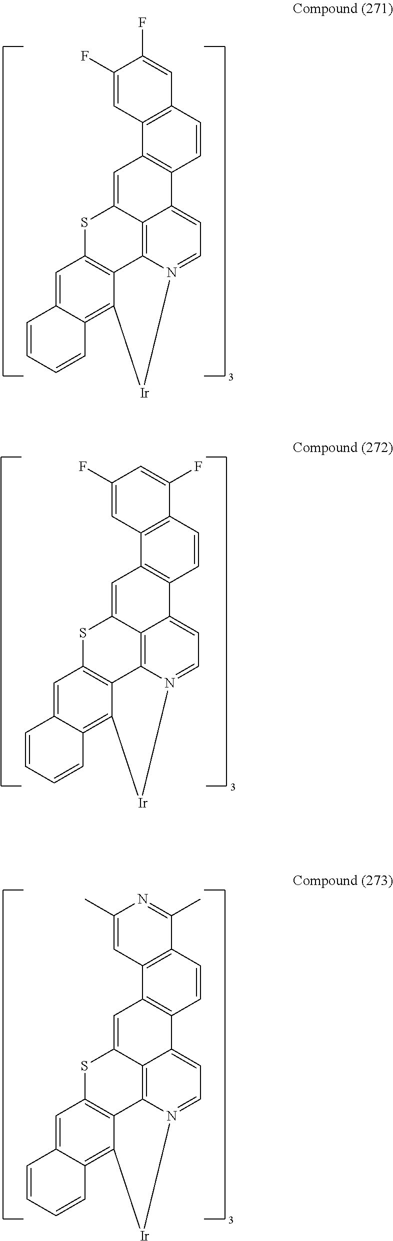

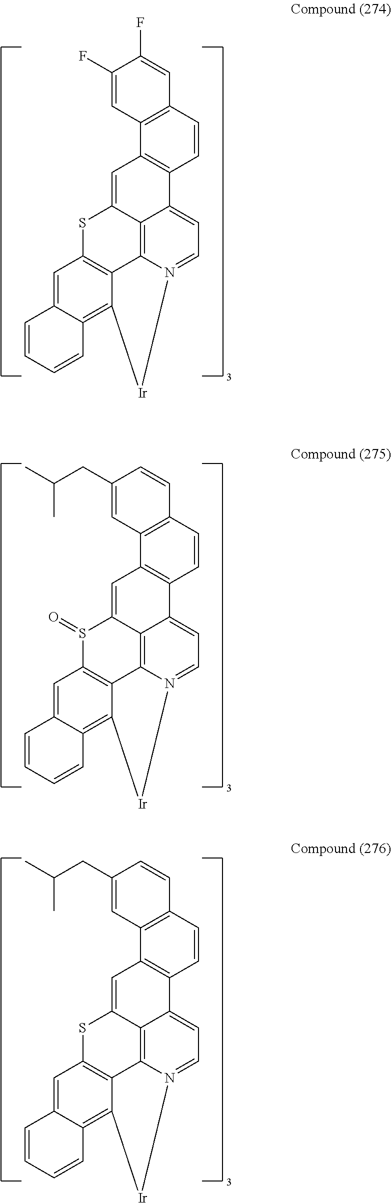

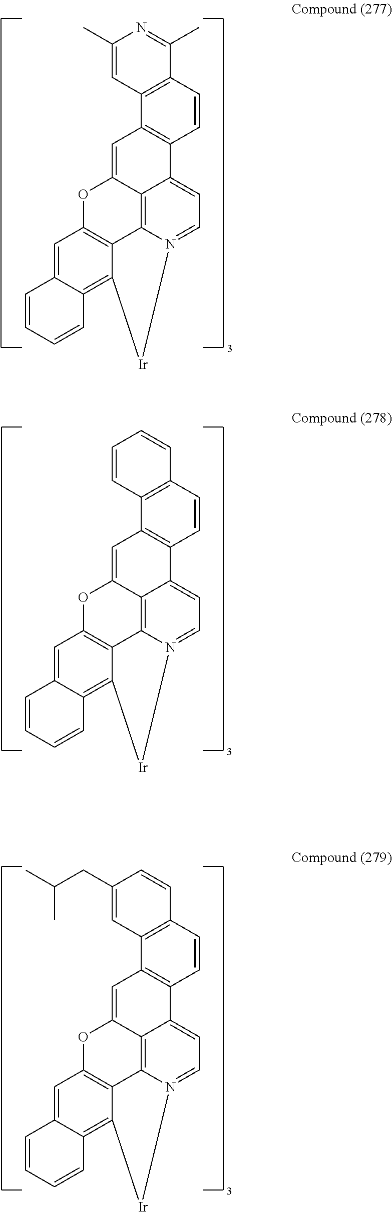

Among the above exemplary compounds, organometallic complexes having three main ligands each having a NIQ ring are stable organometallic complexes due to their high molecular symmetry. Compounds having two main ligands each having a NIQ ring and one ancillary ligand have a low molecular weight and thus can lower the deposition temperature. In particular, organometallic complexes including 2-phenylpyridine as an ancillary ligand are suitable for use as compounds having a good balance between molecular stability and deposition temperature because of appropriate combinations between a main ligand including an appropriate NIQ ring and the ancillary ligand.

In each of the above organometallic complexes, a main ligand including a NIQ ring contributes to light emission, as a result of which the highly robust cyclic ligand suppresses an intramolecular structural change, and the good direction and length of transition dipole moment achieve enhanced oscillator strength, resulting in a reduced full width at half maximum (FWHM) and high light-emission efficiency.

Next, an organic light-emitting element according to this embodiment will be described. The organic light-emitting element according to this embodiment at least includes a first electrode, a second electrode, and an organic compound layer disposed between the electrodes. One of the first electrode and the second electrode is an anode, and the other is a cathode. In the organic light-emitting element according to this embodiment, the organic compound layer may be a single layer or a stack of a plurality of layers as long as the organic compound layer includes a light-emitting layer. When the organic compound layer is a stack of a plurality of layers, the organic compound layer may include, in addition to the light-emitting layer, a hole injection layer, a hole transport layer, an electron blocking layer, a hole/exciton blocking layer, an electron transport layer, an electron injection layer, and the like. The light-emitting layer may be a single layer or a stack of a plurality of layers.

In the organic light-emitting element according to this embodiment, at least one layer of the organic compound layer contains the organometallic complex according to this embodiment. Specifically, the organometallic complex according to this embodiment is contained in any of the light-emitting layer, the hole injection layer, the hole transport layer, the electron blocking layer, the hole/exciton blocking layer, the electron transport layer, the electron injection layer, and the like mentioned above. The organometallic complex according to this embodiment is preferably contained in the light-emitting layer.

In the organic light-emitting element according to this embodiment, when the organometallic complex according to this embodiment is contained in the light-emitting layer, the light-emitting layer may be a layer formed only of the organometallic complex according to this embodiment or a layer formed of the organometallic complex according to this embodiment and other compounds. When the light-emitting layer is a layer formed of the organometallic complex according to this embodiment and other compounds, the organometallic complex according to this embodiment may be used as a host or a guest of the light-emitting layer. The organometallic complex may also be used as an assist material that can be contained in the light-emitting layer. Here, the host refers to a compound accounting for the largest mass proportion among the compounds constituting the light-emitting layer. The guest refers to a compound that accounts for a smaller mass proportion than the host among the compounds constituting the light-emitting layer and that is responsible for main light emission. The assist material refers to a compound that accounts for a smaller mass proportion than the host among the compounds constituting the light-emitting layer and that assists the light emission of the guest. The assist material is also referred to as a second host. The host can also be referred to as a first organic compound, and the assist material as a second organic compound.

When the organometallic complex according to this embodiment is used as a guest, the first organic compound serving as a host has a lowest excited singlet energy and a lowest excited triplet energy higher than those the organometallic complex. The lowest excited triplet energy of the second organic compound serving as an assist material is preferably higher than the lowest excited triplet energy of the organometallic complex and lower than the lowest excited triplet energy of the first organic compound.

When the organometallic complex according to this embodiment is used as a guest of the light-emitting layer, the content of the organometallic complex in the light-emitting layer is preferably 0.01 mass % or more and 20 mass % or less, more preferably 0.1 mass % or more and 10 mass % or less.

The present inventors have conducted various studies and found that when the organometallic complex according to this embodiment is used as a host or a guest of the light-emitting layer, particularly, as a guest of the light-emitting layer, an element that exhibits a light-emission output with high efficiently and high luminance and has very high durability is provided. This light-emitting layer may have a single-layer structure or a multilayer structure, and incorporating a light-emitting material having an emission color different from that of the guest can adjust the emission color. The multilayer structure means a state in which the light-emitting layer and another light-emitting layer are stacked on top of each other. In this case, the emission color of the organic light-emitting element may be white or an intermediate color. In the case of white, a light-emitting layer having an emission color different from the emission color of the light-emitting layer containing the organometallic complex according to this embodiment may be stacked. The light-emitting layers are formed by vapor deposition or coating.

When the organometallic complex according to this embodiment is contained in the light-emitting layer, a layer formed of an organic compound may be disposed between the light-emitting layer and the second electrode and/or between the light-emitting layer and the first electrode. Specifically, the layer formed of an organic compound is a charge transport layer or a charge blocking layer. The charge transport layer and the charge blocking layer each preferably have a higher lowest excited triplet energy than the light-emitting layer, and are each preferably formed of a hydrocarbon compound.

The organometallic complex according to this embodiment can be used as a constituent material of an organic compound layer other than the light-emitting layer constituting the organic light-emitting element according to this embodiment. Specifically, the organometallic complex may be used as a constituent material of, for example, the electron transport layer, the electron injection layer, the hole transport layer, the hole injection layer, or the hole blocking layer.

In addition to the organometallic complex according to this embodiment, conventionally known low-molecular-weight and high-molecular-weight hole injection compounds or hole transport compounds, host materials, assist materials, luminescent compounds, electron injection compounds or electron transport compounds, and the like may optionally be used in combination. Examples of these compounds will be described below.

As hole injection and transport materials, materials that facilitate injection of holes from the anode and that have so high hole mobility that enables injected holes to be transported to the light-emitting layer are preferred. To reduce deterioration of film quality, such as crystallization, in the organic light-emitting element, materials having high glass-transition temperatures are preferred. Examples of low-molecular-weight and high-molecular-weight materials having hole injection and transport properties include triarylamine derivatives, arylcarbazole derivatives, phenylenediamine derivatives, stilbene derivatives, phthalocyanine derivatives, porphyrin derivatives, poly(vinylcarbazole), poly(thiophene), and other conductive polymers. These hole injection and transport materials are also suitable for use in the electron blocking layer. Non-limiting specific examples of compounds usable as hole injection and transport materials are shown below.

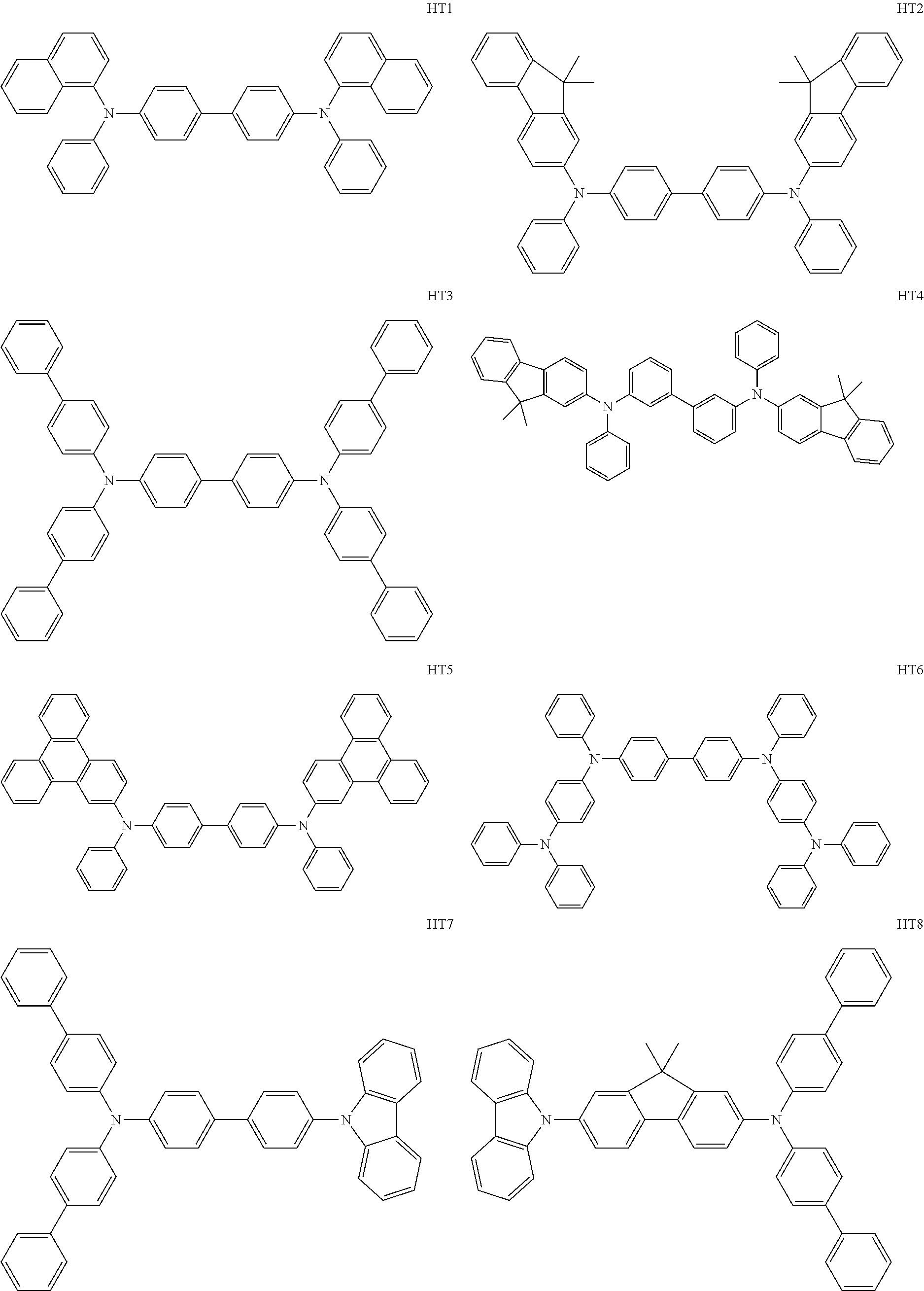

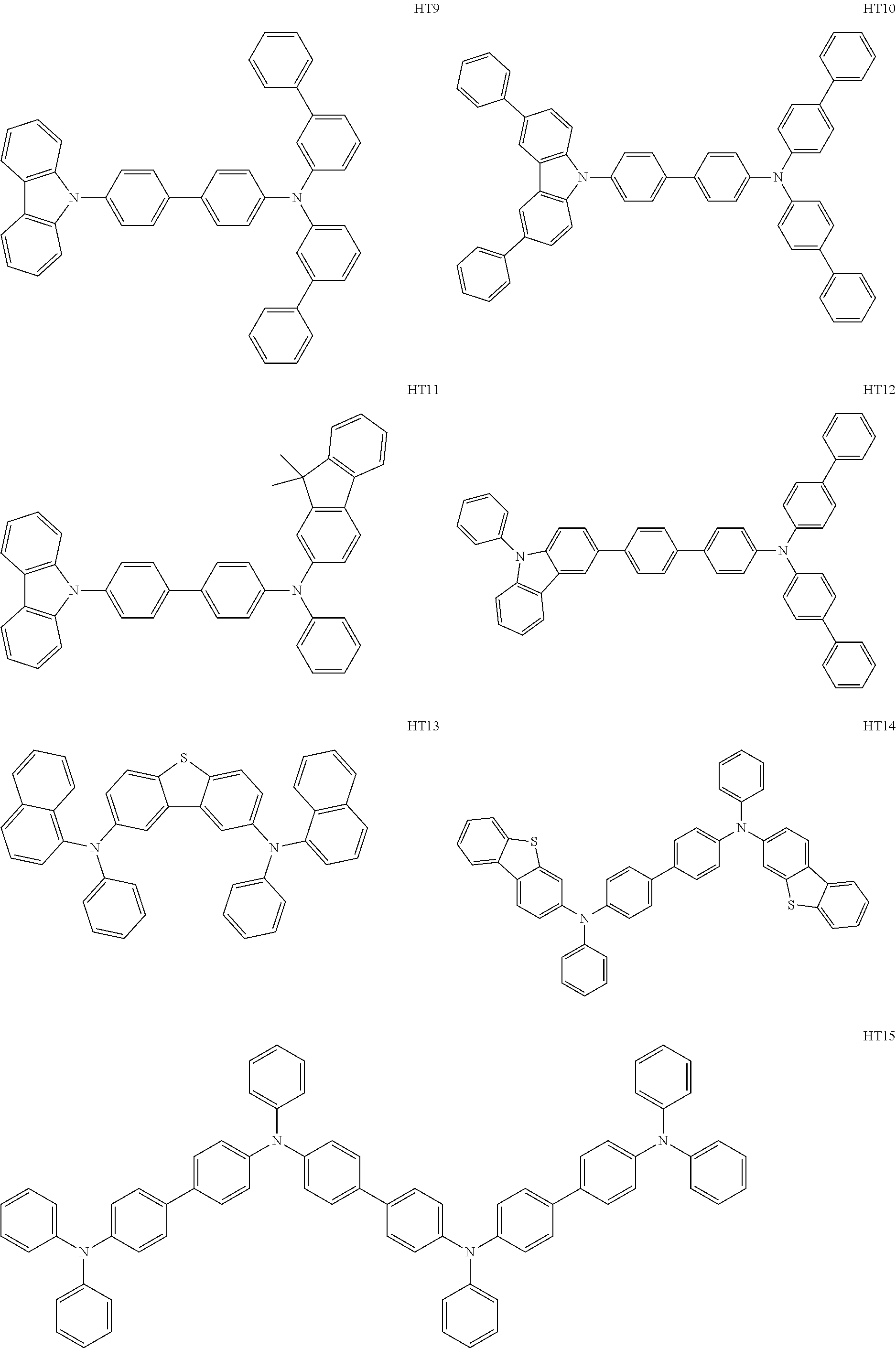

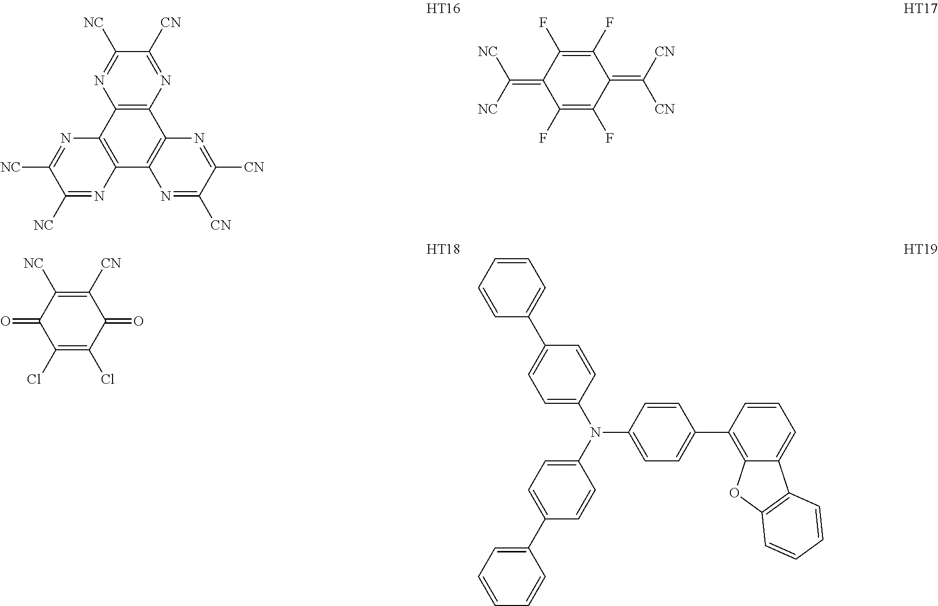

Among the hole injection and transport materials given above, HT16 to HT18 can be used for a layer in contact with the anode to achieve a lower driving voltage. HT16 is widely used in organic light-emitting elements. HT2, HT3, HT4, HT5, HT6, HT10, and HT12 may be used for an organic compound layer adjacent to HT16. A plurality of materials may be used for one organic compound layer.

Examples of light-emitting materials mainly involved in the light-emitting function include fused-ring compounds (e.g., fluorene derivatives, naphthalene derivatives, pyrene derivatives, perylene derivatives, tetracene derivatives, anthracene derivatives, and rubrene), quinacridone derivatives, coumarin derivatives, stilbene derivatives, organic aluminum complexes such as tris(8-quinolinolato)aluminum, iridium complexes, platinum complexes, rhenium complexes, copper complexes, europium complexes, ruthenium complexes, and polymer derivatives such as poly(phenylenevinylene) derivatives, poly(fluorene) derivatives, and poly(phenylene) derivatives.

Non-limiting specific examples of compounds usable as light-emitting materials are shown below.

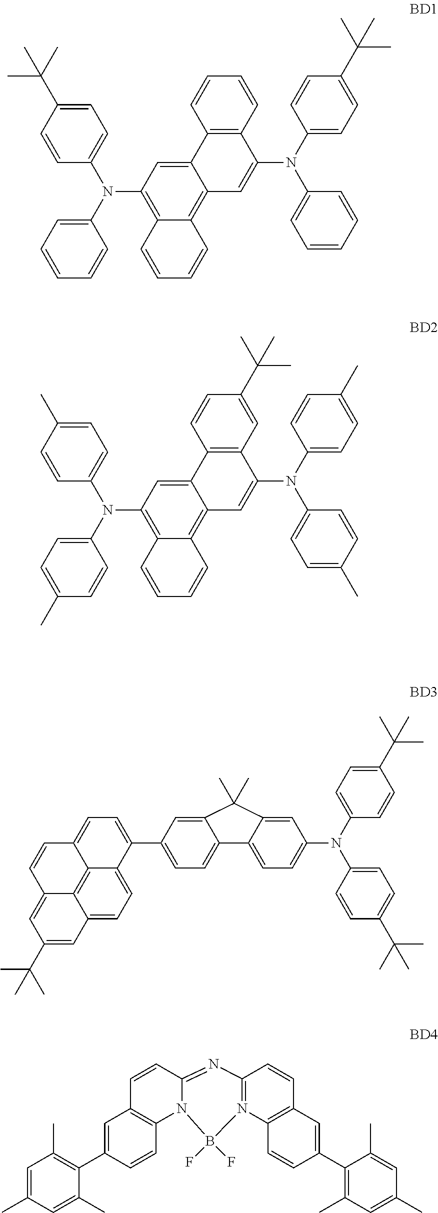

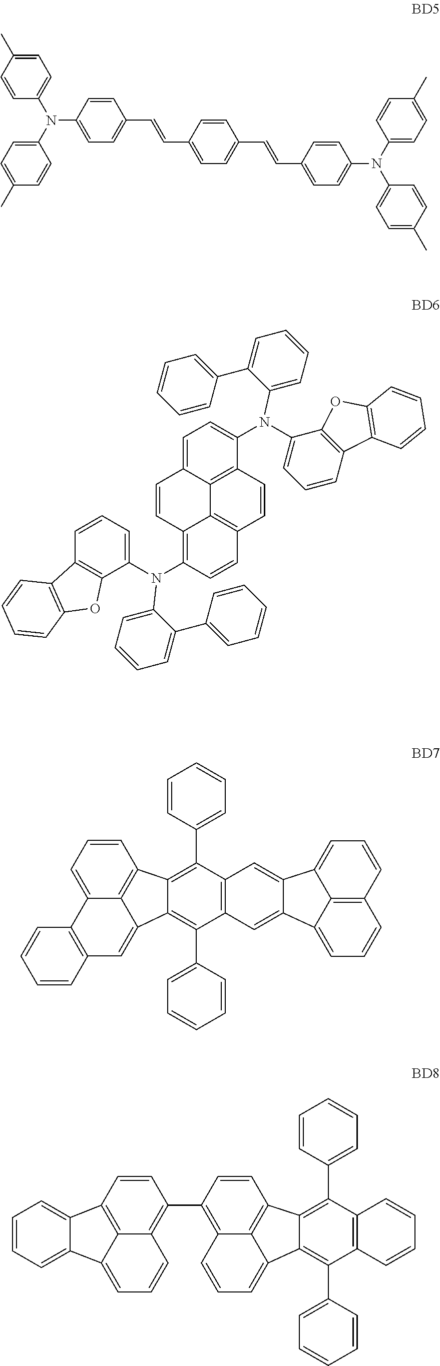

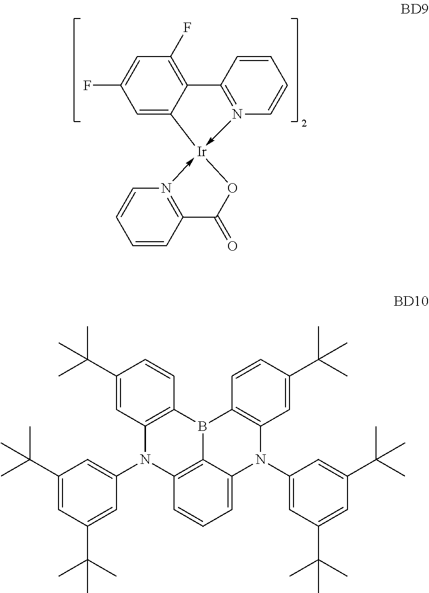

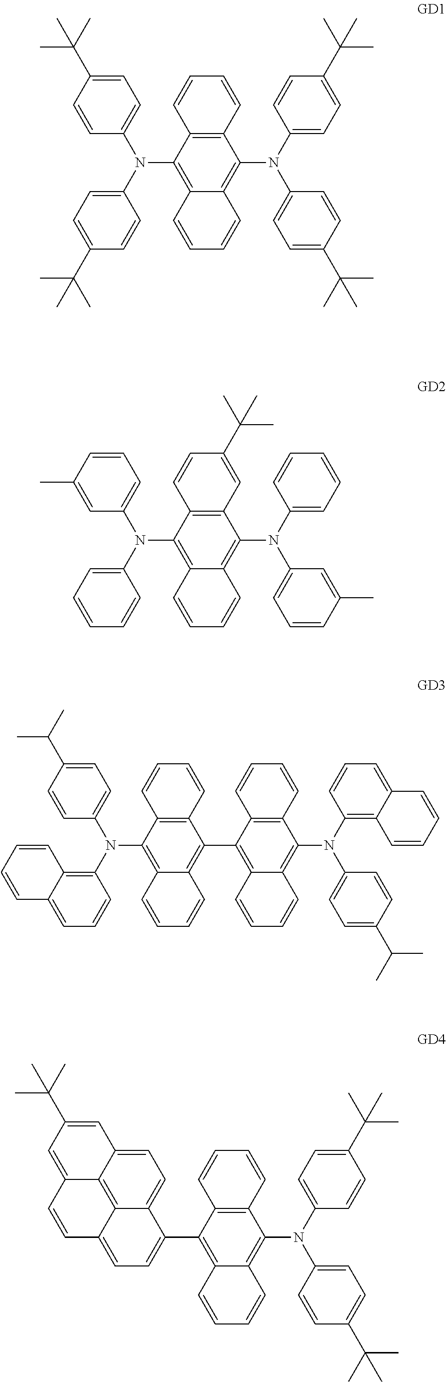

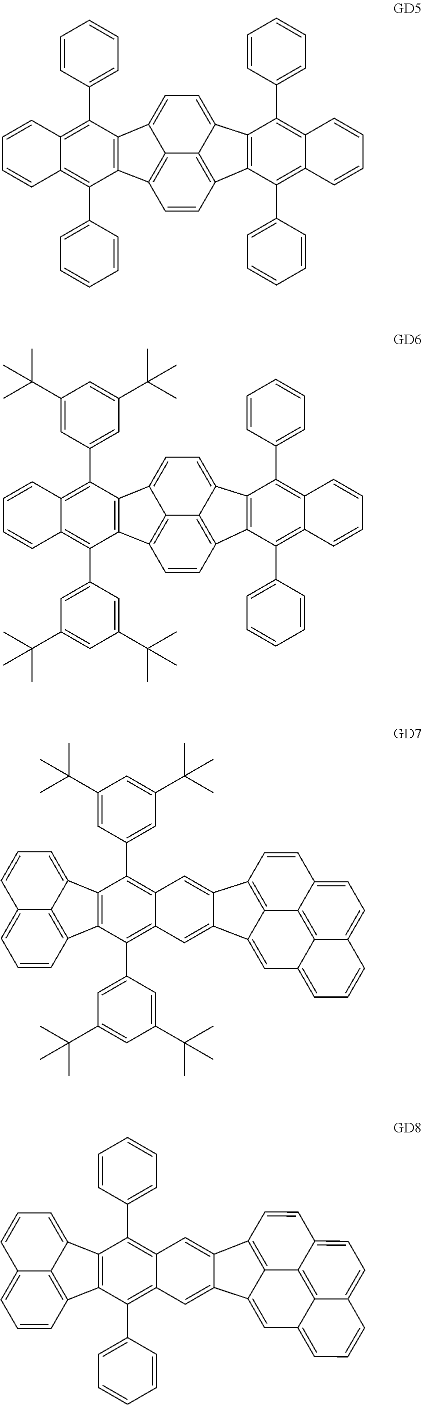

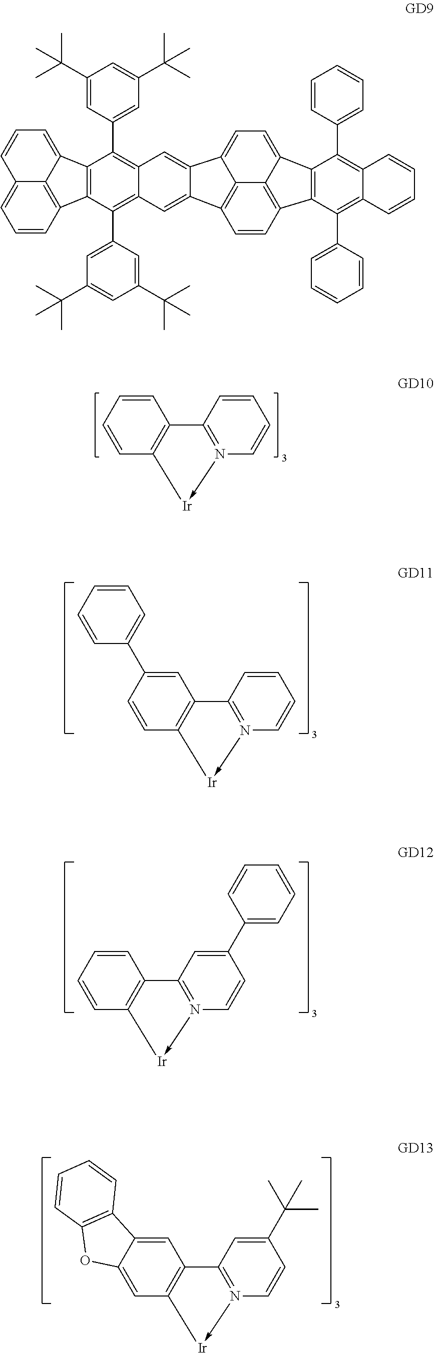

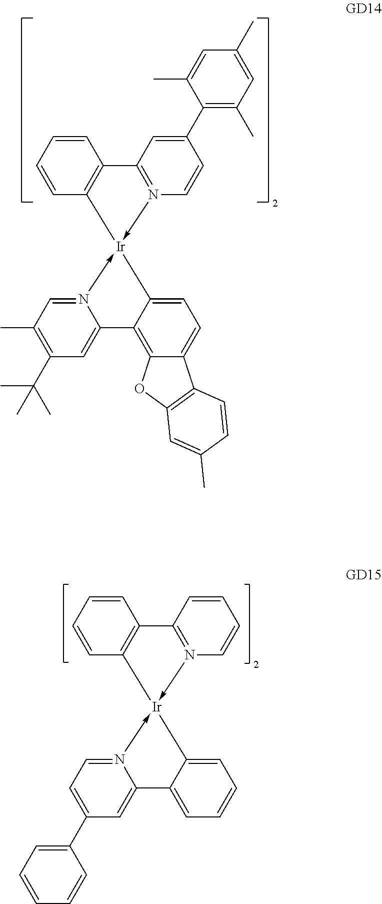

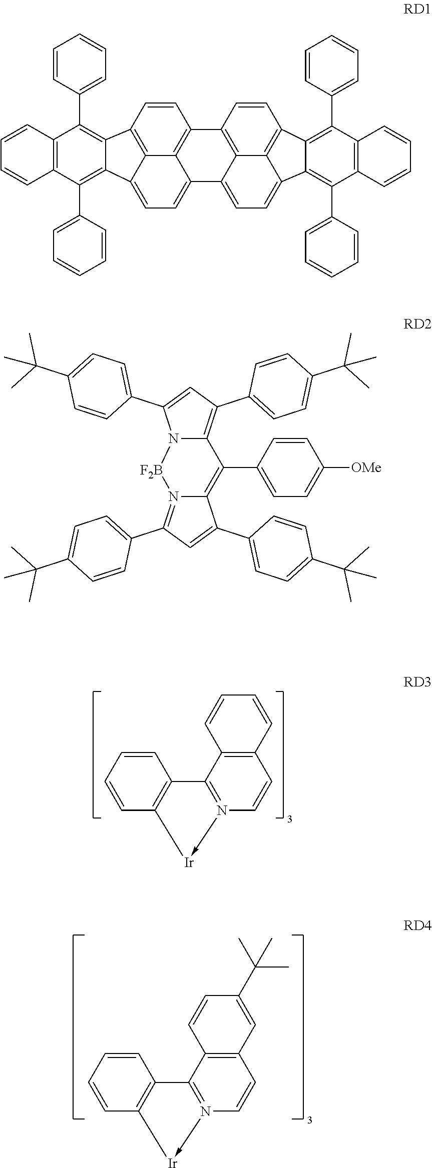

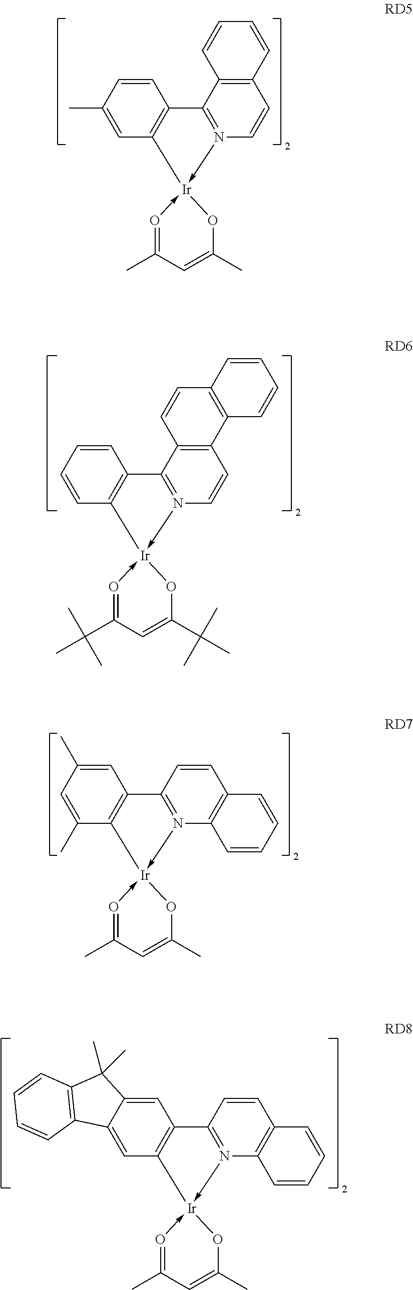

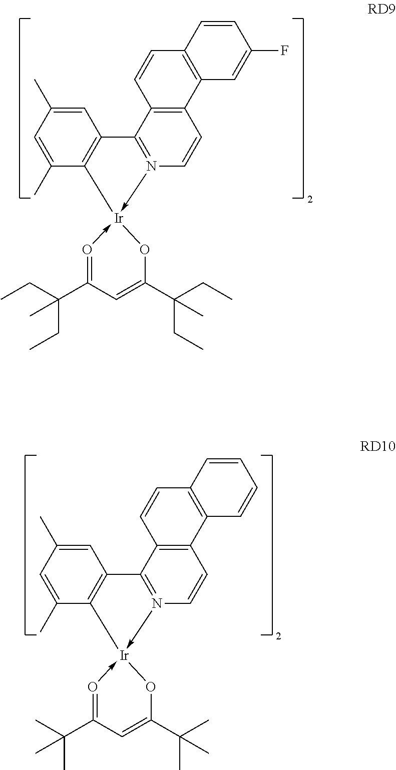

When the light-emitting material is a hydrocarbon compound, a decrease in light-emission efficiency due to exciplex formation and a decrease is color purity due to a change in emission spectrum can be advantageously reduced.

The hydrocarbon compound is a compound consisting of carbon and hydrogen, and among the above exemplary compounds, BD7, BD8, GD5 to GD9, and RD1 are hydrocarbon compounds.

When the light-emitting material is a fused polycyclic compound having a five-membered ring, the light-emitting material is less likely to undergo oxidization due to its high ionization potential, thus providing a highly durable long-life element, which is preferred. Among the above exemplary compounds, BD7, BD8, GD5 to GD9, and RD1 are fused polycyclic compounds having a five-membered ring.

Examples of host or assist materials contained in the light-emitting layer include aromatic hydrocarbon compounds and derivatives thereof, carbazole derivatives, dibenzofuran derivatives, dibenzothiophene derivatives, organic aluminum complexes such as tris(8-quinolinolato)aluminum, and organic beryllium complexes.

Non-limiting specific examples of compounds usable as host or assist materials contained in the light-emitting layer are shown below.

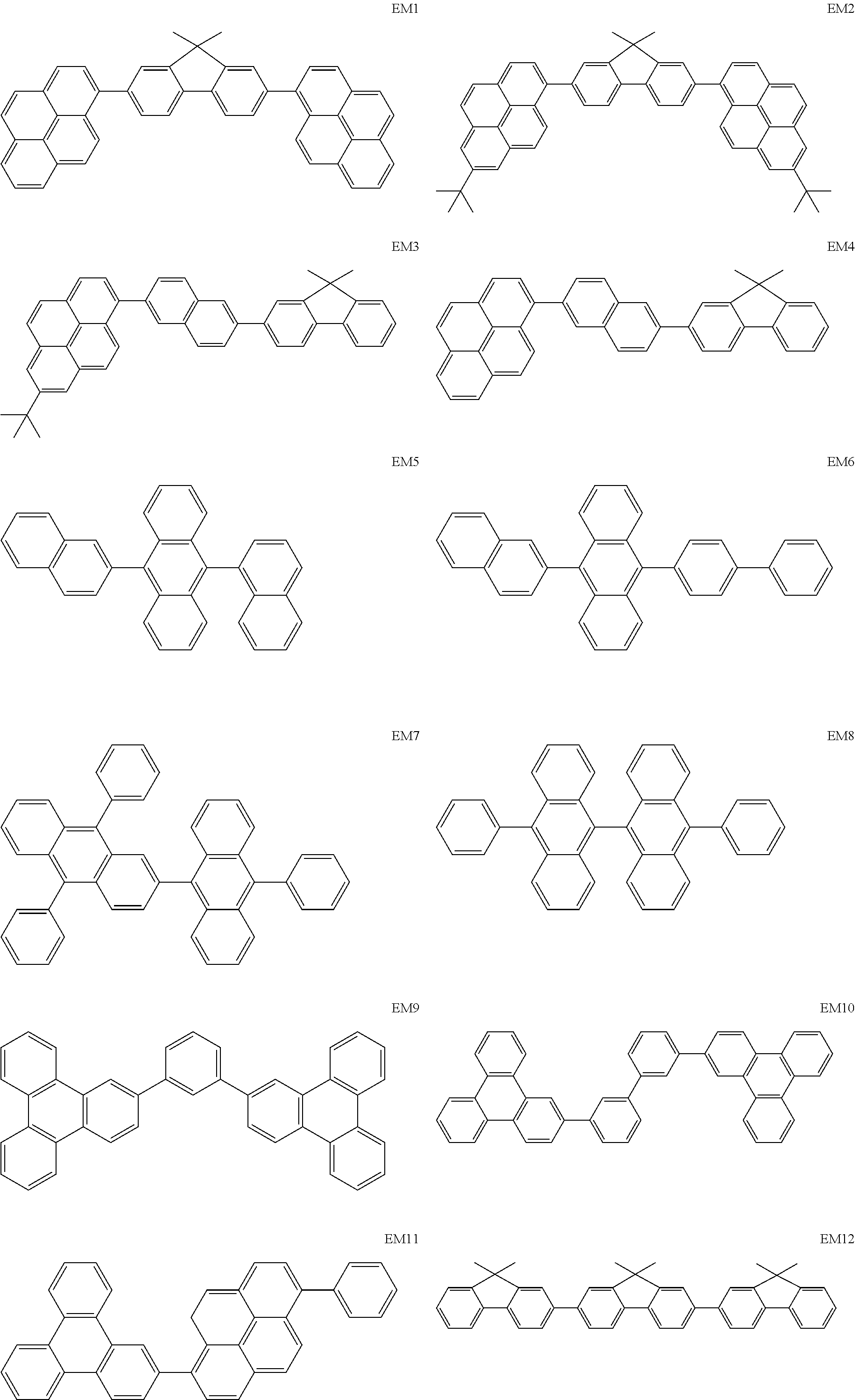

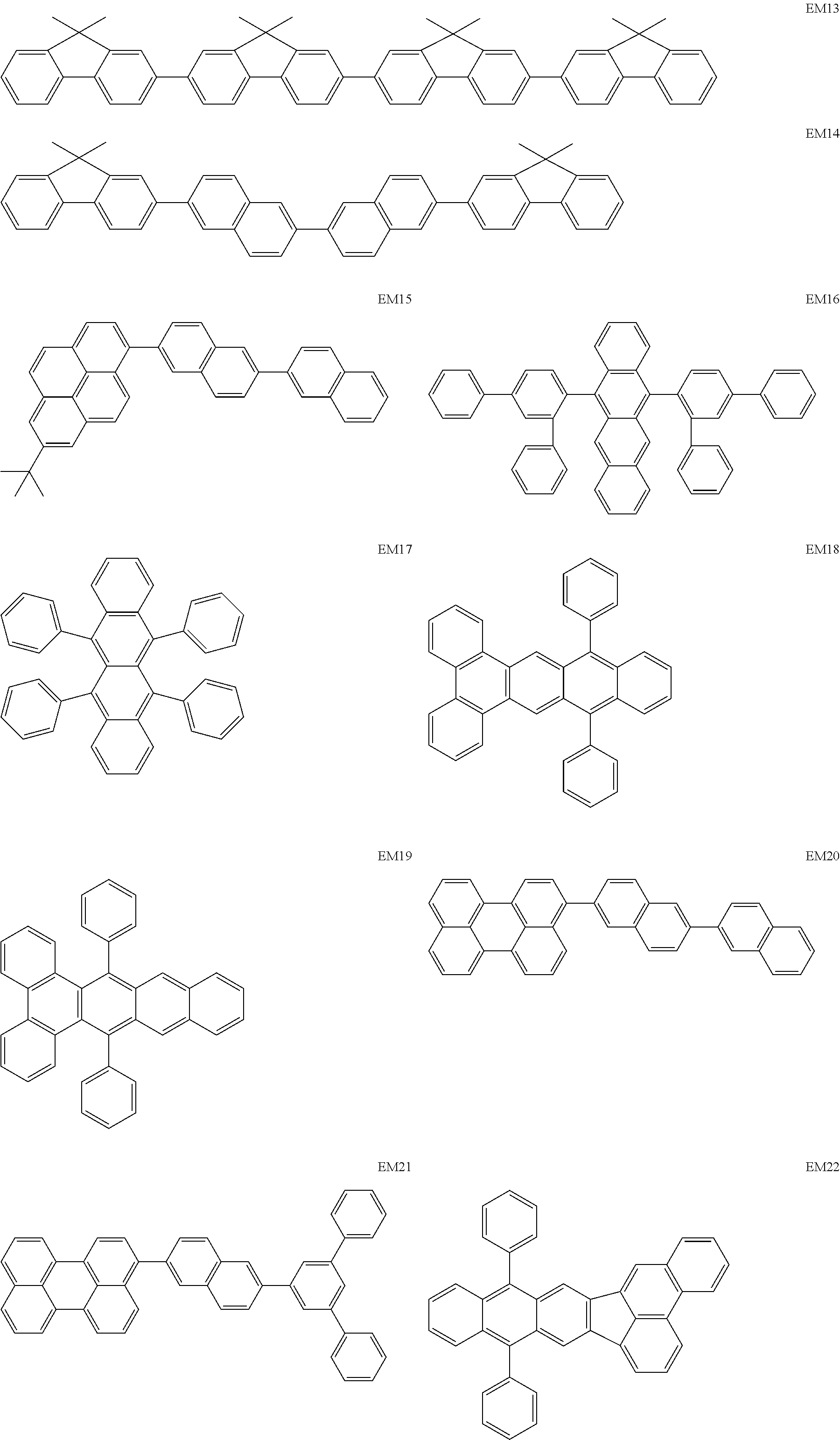

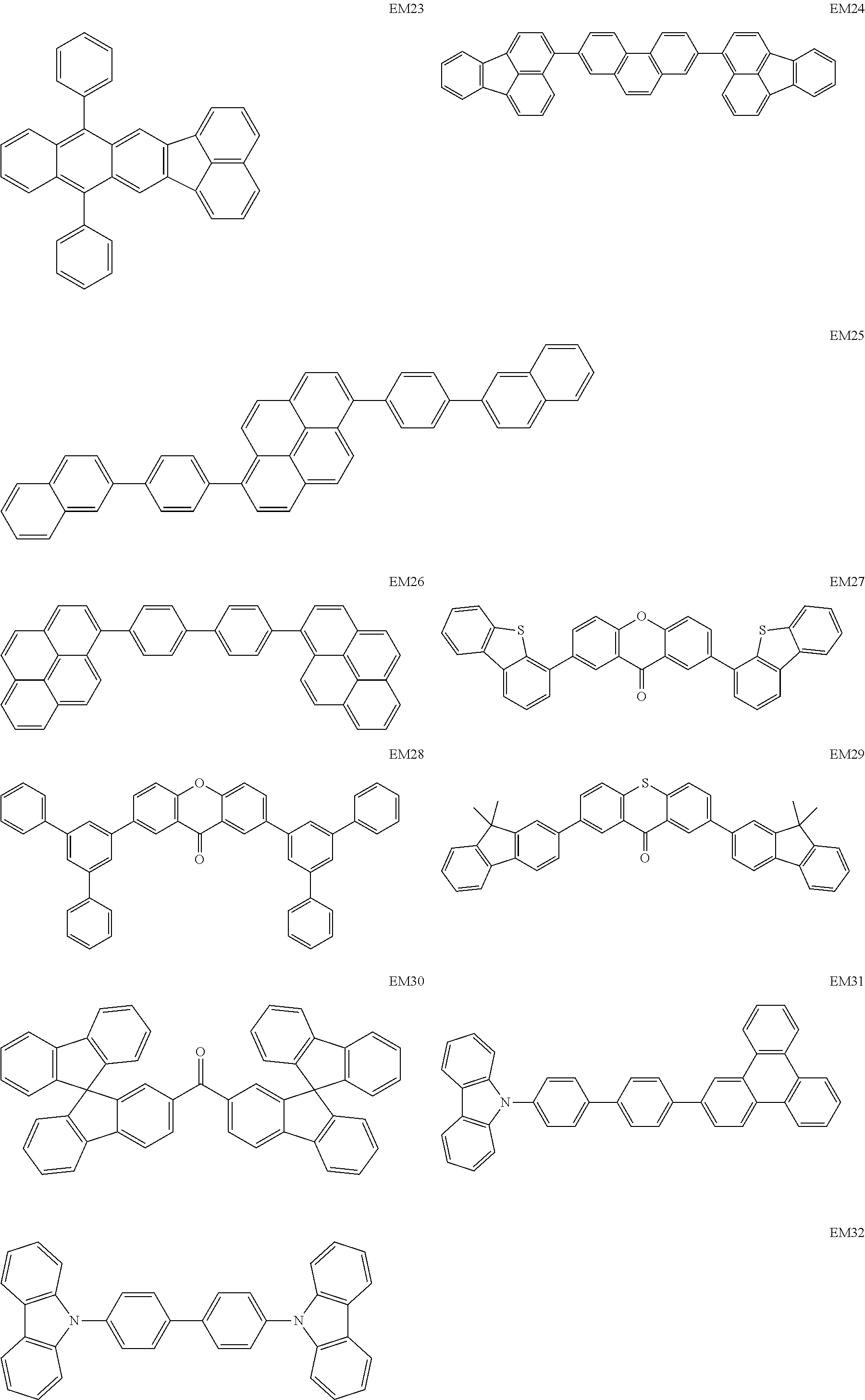

When the host material is a hydrocarbon compound, the organometallic complex according to this embodiment advantageously can easily trap electrons and holes, thus providing higher efficiency. Among the above exemplary compounds, EM1 to EM26 are hydrocarbon compounds.

Any electron transport material capable of transporting electrons injected from the cathode to the light-emitting layer can be freely selected in consideration of, for example, the balance with the hole mobility of a hole transport material. Examples of materials capable of transporting electrons include oxadiazole derivatives, oxazole derivatives, pyrazine derivatives, triazole derivatives, triazine derivatives, quinoline derivatives, quinoxaline derivatives, phenanthroline derivatives, organic aluminum complexes, and fused-ring compounds (e.g., fluorene derivatives, naphthalene derivatives, chrysene derivatives, and anthracene derivatives). These electron transport materials are also suitable for use for the hole blocking layer.

Non-limiting specific examples of compounds usable as electron transport materials are shown below.

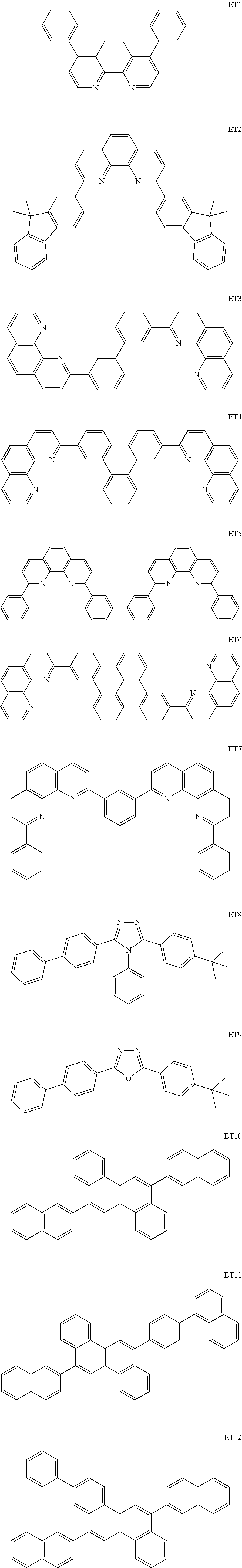

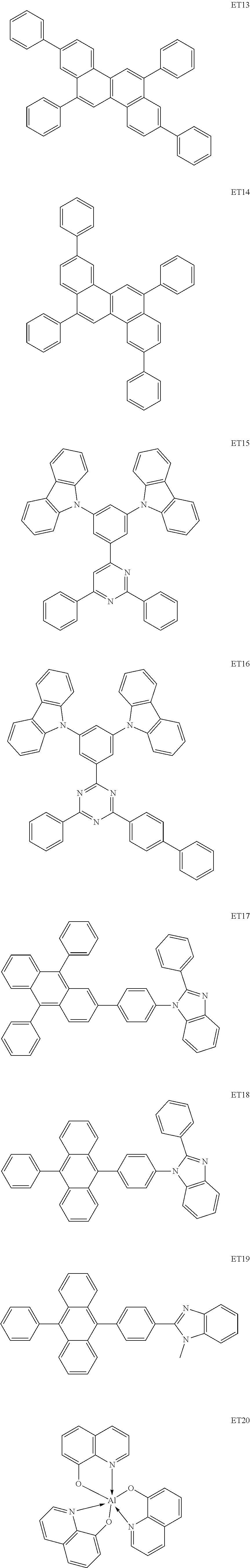

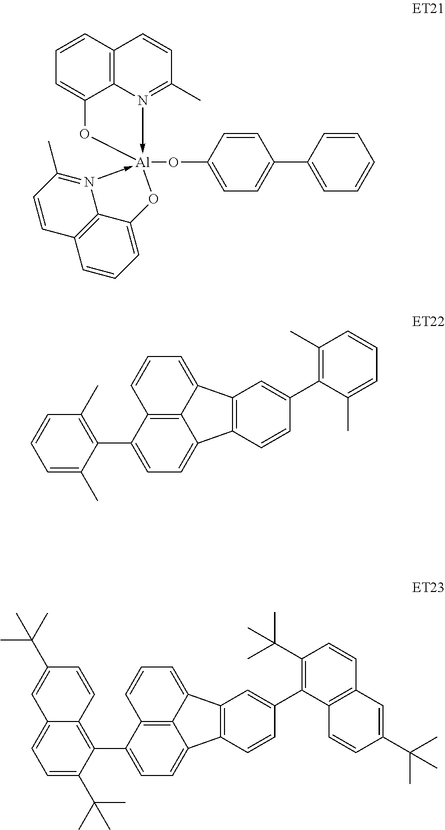

Any electron injection material capable of readily injecting electrons from the cathode can be freely selected in consideration of, for example, the balance with hole injectability. An n-type dopant and a reducing dopant are also contained as organic compounds. Examples include alkali metal-containing compounds such as lithium fluoride, lithium complexes such as lithium quinolinol, benzimidazolidene derivatives, imidazolidene derivatives, fulvalene derivatives, and acridine derivatives.

These can also be used in combination with the electron transport materials above.

For the organic compound layers (e.g., the hole injection layer, the hole transport layer, the electron blocking layer, the hole blocking layer, the electron transport layer, and the electron injection layer) other than the light-emitting layer, a dry process such as vacuum deposition, ionized deposition, sputtering, or plasma can be used. Instead of the dry process, a wet process involving dissolving a material in an appropriate solvent and forming a layer by a known coating method (e.g., spin coating, dipping, a casting method, an LB method, or an ink jet method) can also be used.

When the layers are formed by, for example, vacuum deposition or solution coating, the layers are unlikely to undergo crystallization or the like and are highly stable over time. When a coating method is used for film formation, an appropriate binder resin can be used in combination to form a film.

Examples of the binder resin include polyvinylcarbazole resins, polycarbonate resins, polyester resins, ABS resins, acrylic resins, polyimide resins, phenol resins, epoxy resins, silicone resins, and urea resins, but are not limited thereto.

These binder resins may be used alone as a homopolymer or copolymer or may be used as a mixture of two or more. In addition, known additives such as plasticizers, antioxidants, and UV absorbers may be used in combination as required.

### Ink Composition

The organometallic complex according to this embodiment has good solubility in organic solvents, and thus can be used in the form of a light-emitting ink composition. Using the ink composition enables the layers formed of organic compounds constituting the organic light-emitting element according to this embodiment, particularly, the light-emitting layer, to be formed by a coating method, so that a large-area element can be easily produced at a relatively low cost. The ink composition contains the organometallic complex according to this embodiment and a solvent. Examples of the solvent include toluene, xylene, mesitylene, dioxane, methylnaphthalene, tetrahydrofuran, diglyme, 1,2-dichlorobenzene, and 1,2-dichloropropane. These organic solvents can be used alone or in combination of two or more. Of these, those having an appropriate evaporation rate, specifically, organic solvents having a boiling point of about 70° C. to 200° C. are preferably used because a thin film having a uniform thickness is easily provided.

The light-emitting ink composition according to this embodiment may further contain a compound that serves as an additive. Specifically, the compound is selected according to the function of an organic compound layer in which the organometallic complex is contained, and examples include known host or assist materials, hole transport materials, light-emitting materials, and electron transport materials.

The content of the organometallic complex in the light-emitting ink composition is preferably 0.05 mass % or more and 20 mass % or less, more preferably 0.1 mass % or more and 5 mass % or less.

By using the ink composition, the light-emitting layer can be formed by, for example, a spin coating method, a bar coating method, a slit coating method, an ink-jet method, a nozzle coating method, a casting method, or a gravure printing method.

### Configuration of Organic Light-Emitting Element

The organic light-emitting element is provided by forming an insulating layer, a first electrode, an organic compound layer, and a second electrode on a substrate. On the second electrode, a protective layer, a color filter, a microlens, etc. may be disposed. When the color filter is disposed, a planarization layer may be disposed between the color filter and the protective layer. The planarization layer may be formed of, for example, an acrylic resin. This also applies to the case where the planarization layer is disposed between the color filter and the microlens.

Members of the organic light-emitting element other than the organic compound layer will be described below.

### Substrate

Examples of the substrate include quartz, glass, silicon wafers, resins, and metals. The substrate may have switching elements, such as transistors, and wiring lines disposed thereon, and may have an insulating layer disposed thereon. The insulating layer may be made of any material as long as a contact hole can be formed therein so as to allow formation of a wiring line connecting to the first electrode and insulation from unconnected wiring lines can be provided. For example, resins such as polyimide, silicon oxide, silicon nitride, and the like can be used.

### Electrode

One of the first electrode and the second electrode is an anode, and the other is a cathode. When an electric field is applied in a direction in which the organic light-emitting element emits light, one of the electrodes at a higher potential is the anode, and the other is the cathode. Stated another way, one of the electrodes that supplies holes to the light-emitting layer is the anode, and the other electrode that supplies electrons to the light-emitting layer is the cathode.

The constituent material of the anode preferably has as high a work function as possible. For example, elemental metals such as gold, platinum, silver, copper, nickel, palladium, cobalt, selenium, vanadium, and tungsten, mixtures containing these metals, alloys obtained by combining these metals, and metal oxides such as tin oxide, zinc oxide, indium oxide, indium tin oxide (ITO), and indium zinc oxide can be used. Conductive polymers such as polyaniline, polypyrrole, and polythiophene can also be used.

These electrode materials may be used alone or in combination of two or more. The anode may be composed of a single layer or a plurality of layers.

When the anode is used as a reflection electrode, for example, chromium, aluminum, silver, titanium, tungsten, molybdenum, an alloy thereof, or a stack thereof can be used. These materials can also be used to function as a reflective film that does not have a role of an electrode. When the anode is used as a transparent electrode, it may be, for example, but not necessarily, a transparent conductive layer made of an oxide such as indium tin oxide (ITO) or indium zinc oxide. The electrode can be formed by photolithography.

The constituent material of the cathode preferably has a low work function. Examples of such materials include alkali metals such as lithium; alkaline earth metals such as calcium; elemental metals such as aluminum, titanium, manganese, silver, lead, and chromium; and mixtures thereof. Alloys obtained by combining these elemental metals can also be used. For example, magnesium-silver, aluminum-lithium, aluminum-magnesium, silver-copper, and zinc-silver alloys can be used. Metal oxides such as indium tin oxide (ITO) can also be used. These electrode materials may be used alone or in combination of two or more. The cathode may be composed of a single layer or a plurality of layers. In particular, silver is preferably used, and a silver alloy is more preferred to reduce aggregation of silver. As long as aggregation of silver can be reduced, the content ratio in the alloy is not limited. For example, the ratio of silver to other metals may be, for example, 1:1 or 3:1.

The cathode is not particularly limited; a conductive layer formed of an oxide such as ITO may be used to provide a top-emission element, or a reflection electrode formed of aluminum (Al) or the like may be used to provide a bottom-emission element. The method of forming the cathode is not particularly limited, and the use of DC sputtering or AC sputtering is more preferred because good film coverage can be achieved and the resistance tends to decrease.

### Protective Layer

A protective layer may be disposed on the second electrode. For example, a glass member provided with a moisture absorbent can be bonded onto the second electrode to reduce the entry of, for example, water into the organic compound layer, thereby reducing the occurrence of display failure. In another embodiment, a passivation film made of silicon nitride or the like may be disposed on the second electrode to reduce the entry of, for example, water into the organic compound layer. For example, the protective layer may be formed in a manner that after the formation of the second electrode, the resultant is conveyed to another chamber without breaking the vacuum, and a silicon nitride film having a thickness of 2 μm is formed by CVD. After the film formation by CVD, atomic layer deposition (ALD) may be performed to form a protective layer. The material of the film formed by ALD is not limited and may be, for example, silicon nitride, silicon oxide, or aluminum oxide. On the film formed by ALD, silicon nitride may further be formed by CVD. The film formed by ALD may have a smaller thickness than the film formed by CVD. Specifically, the thickness may be 50% or less, or even 10% or less.

### Color Filter

A color filter may be disposed on the protective layer. For example, a color filter may be formed on another substrate so as to correspond to the size of the organic light-emitting element and bonded to the substrate having the organic light-emitting element disposed thereon. Alternatively, a color filter may be patterned on the above-described protective layer by photolithography. The color filter may be made of a polymer.

### Planarization Layer

A planarization layer may be disposed between the color filter and the protective layer. The planarization layer is disposed for the purpose of reducing unevenness of the underlying layer. The planarization layer may be referred to as a material resin layer without limiting the purpose. The planarization layer may be formed of an organic compound. The organic compound may have a low molecular weight or a high molecular weight, and preferably has a high molecular weight.

The planarization layer may be disposed on opposite surfaces of the color filter, and the constituent materials thereof may be the same or different. Specific examples include polyvinylcarbazole resins, polycarbonate resins, polyester resins, ABS resins, acrylic resins, polyimide resins, phenol resins, epoxy resins, silicone resins, and urea resins.

### Microlens

An organic light-emitting apparatus may include, on its light-emitting side, an optical member such as a microlens. The microlens can be formed of, for example, an acrylic resin or an epoxy resin. The microlens may be used to increase the amount of light extracted from the organic light-emitting apparatus and to control the direction of the extracted light. The microlens may have a hemispherical shape. In the case of a hemispherical shape, among tangents to the hemisphere, there is a tangent parallel to the insulating layer, and the point of contact between this tangent and the hemisphere is the vertex of the microlens. The vertex of the microlens can be determined in the same manner in any sectional view. That is, among tangents to the semicircle of the microlens in a sectional view, there is a tangent parallel to the insulating layer, and the point of contact between this tangent and the semicircle is the vertex of the microlens.

The midpoint of the microlens can also be defined. In a section of the microlens, a line segment from one end point to the other end point of the arc is imagined, and the midpoint of the line segment can be referred to as the midpoint of the microlens. The section used to determine the vertex and the midpoint may be a section perpendicular to the insulating layer.

### Opposite Substrate

An opposite substrate may be disposed on the planarization layer. The opposite substrate is disposed at a position opposite to the above-described substrate and thus is referred to as the opposite substrate. The constituent material of the opposite substrate may be the same as that of the above-described substrate. When the above-described substrate is a first substrate, the opposite substrate may be a second substrate.

Configuration of Apparatus Including Organic Light-Emitting Element

### Pixel Circuit

A light-emitting apparatus including the organic light-emitting element according to this embodiment may include a pixel circuit connected to the organic light-emitting element. The pixel circuit may be an active matrix-type circuit which independently controls the light emission of a first organic light-emitting element and a second organic light-emitting element. The active matrix-type circuit may be voltage programmed or current programmed. A drive circuit includes the pixel circuit for each pixel. The pixel circuit may include an organic light-emitting element, a transistor that controls the emission luminance of the organic light-emitting element, a transistor that controls the timing of light emission, a capacitor that holds the gate voltage of the transistor that controls the emission luminance, and a transistor for providing a connection to GND not through the organic light-emitting element.

The light-emitting apparatus has a display region and a peripheral region disposed around the display region. The display region includes the pixel circuit, and the peripheral region includes a display control circuit. The mobility of the transistor constituting the pixel circuit may be lower than the mobility of a transistor constituting the display control circuit.

The gradient of the current-voltage characteristics of the transistor constituting the pixel circuit may be smaller than the gradient of the current-voltage characteristics of the transistor constituting the display control circuit. The gradient of the current-voltage characteristics can be determined on the basis of, what is called, Vg-Ig characteristics.

The transistor constituting the pixel circuit is a transistor connected to an organic light-emitting element such as the first organic light-emitting element.

### Pixel

The light-emitting apparatus including the organic light-emitting element according to this embodiment includes a plurality of pixels. Each pixel includes subpixels that emit light of colors different from each other. The subpixels may respectively have, for example, R, G, and B emission colors.

In the pixel, a region also referred to as a pixel aperture emits light. This region is the same as the first region. The size of the pixel aperture may be 15 μm or less and 5 μm or more. More specifically, the size may be, for example, 11 μm, 9.5 μm, 7.4 μm, or 6.4 μm. The distance between the subpixels may be 10 μm or less, specifically, 8 μm, 7.4 μm, or 6.4 μm.

The pixels may be in a known arrangement when viewed in plan. For example, the arrangement may be the stripe arrangement, the delta arrangement, the PenTile arrangement, or the Bayer arrangement. The shape of the subpixel in plan view may be any known shape. Examples include quadrangles, such as rectangles and rhombuses, and hexagons. It is appreciated that shapes that are not exactly rectangles but are similar to rectangles are also regarded as rectangles. The shape of the subpixel and the pixel arrangement can be used in combination.

### Applications of Organic Light-Emitting Element

The organic light-emitting element according to this embodiment can be used as a constituent member of a display apparatus or a lighting apparatus. Other applications include an exposure light source in an electrophotographic image-forming apparatus, a backlight in a liquid crystal display, and a light-emitting apparatus including a white light source with a color filter.

The display apparatus may be an image information processor that includes an image input unit to which image information from an area CCD, a linear CCD, a memory card, or the like is input, includes an information-processing unit configured to process the input information, and displays the input image on a display unit.

The display unit of an image pickup apparatus or an ink-jet printer may have a touch panel function. The touch panel function may be activated by any system, such as an infrared system, an electrostatic capacitive system, a resistive film system, or an electromagnetic induction system. The display apparatus may also be used as a display unit of a multifunctional printer.

Next, the display apparatus according to this embodiment will be described with reference to the drawings.

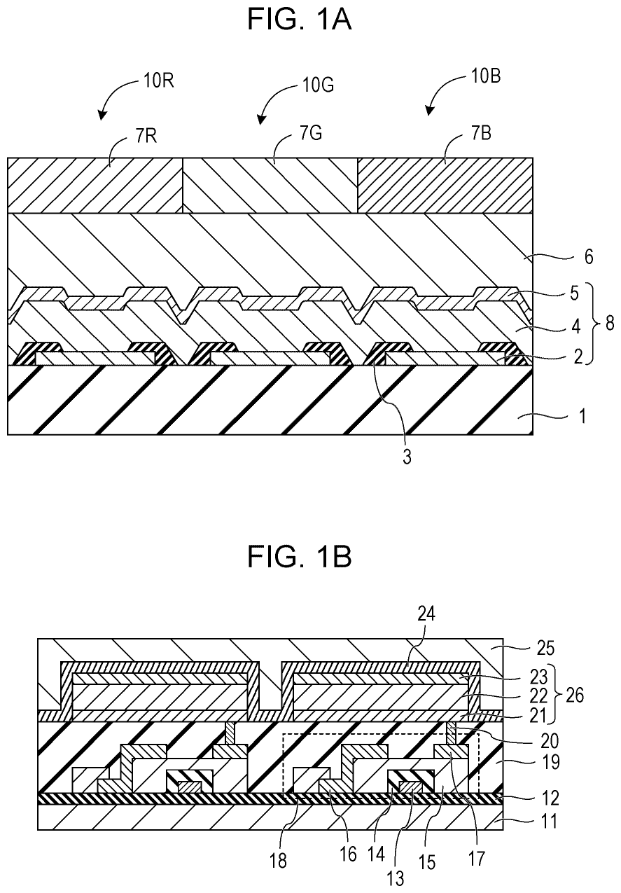

FIG. 1A and FIG. 1B are schematic sectional views each showing an example of a display apparatus including the organic light-emitting element according to this embodiment and a transistor connected to the organic light-emitting element.

FIG. 1A is an example of a pixel that is a component of the display apparatus according to this embodiment. The pixel includes subpixels 10. The subpixels are divided into 10R, 10G, and 10B according to their light emission. The emission color may be distinguished on the basis of the wavelength of light emitted from a light-emitting layer, or light emitted from the subpixels may undergo selective transmission or color conversion through a color filter or the like. Each subpixel includes, on an interlayer insulating layer 1, a first electrode 2 serving as a reflection electrode, an insulating layer 3 that covers the edge of the first electrode 2, an organic compound layer 4 that covers the first electrode 2 and the insulating layer 3, a second electrode 5, a protective layer 6, and a color filter 7. The first electrode 2, the organic compound layer 4, and the second electrode 5 constitute an organic light-emitting element.

The interlayer insulating layer 1 may include a transistor and a capacitor element below or inside the interlayer insulating layer 1. The transistor and the first electrode 2 may be electrically connected to each other through a contact hole (not illustrated) or the like.

The insulating layer 3 is also referred to as a bank or a pixel-separating film. The insulating layer 3 is disposed so as to cover the edge of the first electrode 2 and surround the first electrode 2. A portion in which the insulating layer 3 is not disposed is in contact with the organic compound layer 4 and serves as a light-emitting region.

The second electrode 5 may be a transparent electrode, a reflection electrode, or a semitransparent electrode.

The protective layer 6 reduces permeation of water into the organic compound layer 4. Although the protective layer 6 is illustrated as a single layer, it may be constituted by a plurality of layers. The layers may be constituted by an inorganic compound layer and an organic compound layer.

The color filter 7 is divided into 7R, 7G, and 7B according to their color. The color filter may be formed on a planarization film (not illustrated). A resin protective layer (not illustrated) may be disposed on the color filter. The color filter may be formed on the protective layer 6. The color filter may be bonded after being formed on an opposite substrate such as a glass substrate.

A display apparatus in FIG. 1B includes an organic light-emitting element 26 and a TFT 18, which is an example of a transistor. Specifically, an insulating layer 12 is disposed on a substrate 11 made of glass, silicon, or the like, and the TFT 18 including a gate electrode 13, a gate insulating film 14, a semiconductor layer 15, a drain electrode 16, and a source electrode 17 is disposed on the insulating layer 12. An insulating film 19 is disposed over the TFT 18, and an anode 21 constituting the organic light-emitting element 26 and the source electrode 17 are connected to each other through a contact hole 20 extending through the insulating film 19.

The electrodes (the anode 21 and a cathode 23) included in the organic light-emitting element 26 and the electrodes (the source electrode 17 and the drain electrode 16) included in the TFT 18 need not necessarily be electrically connected to each other in the manner illustrated in FIG. 1B. It is only required that either the anode 21 or the cathode 23 be electrically connected to either the source electrode 17 or the drain electrode 16. TFT stands for a thin film transistor.

A first protective layer 24 and a second protective layer 25 for reducing deterioration of the organic light-emitting element are disposed over the cathode 23.

The emission luminance of the organic light-emitting element 26 according to this embodiment is controlled by the TFT 18, and disposing a plurality of the organic light-emitting elements 26 in a screen enables a display of an image with different emission luminances.

Although a transistor is used as a switching element in the display apparatus in FIG. 1B, another switching element may be used instead.

The transistor used in the display apparatus in FIG. 1B may be not only a TFT including a substrate and an active layer on an insulating surface of the substrate but also a transistor obtained using a single-crystal silicon wafer. The active layer may be formed of non-single-crystal silicon such as amorphous silicon or microcrystalline silicon or a non-single-crystal oxide semiconductor such as indium zinc oxide or indium gallium zinc oxide.

Alternatively, a transistor formed of low-temperature polysilicon or an active matrix driver formed on a substrate such as a Si substrate may also be used. The phrase “on a substrate” may also be referred to as “in a substrate”. Whether a transistor is provided in the substrate or a TFT is used is chosen depending on the size of the display unit. For example, when the display unit has a size of about 0.5 inches, the organic light-emitting element is preferably disposed on a Si substrate. The phrase “formed in a substrate” means producing a transistor by processing a substrate itself, such as a Si substrate. That is, having a transistor in a substrate can also mean that the substrate and the transistor are integrally formed.

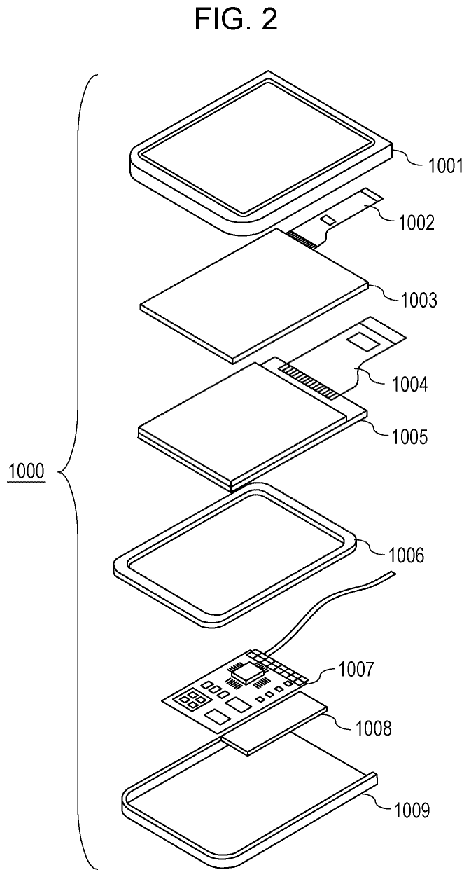

FIG. 2 is a schematic view showing an example of the display apparatus according to this embodiment. A display apparatus 1000 includes an upper cover 1001, a lower cover 1009, and a touch panel 1003, a display panel 1005, a frame 1006, a circuit board 1007, and a battery 1008 disposed between the covers. Flexible print circuits (FPCs) 1002 and 1004 are connected to the touch panel 1003 and the display panel 1005, respectively. A transistor is printed on the circuit board 1007. The battery 1008 may be omitted if the display apparatus is not a mobile device. If the display apparatus is a mobile device, the battery 1008 may be disposed in another position.

The display apparatus according to this embodiment may include red, green, and blue color filters. The red, green, and blue color filters may be disposed in the delta arrangement.

The display apparatus according to this embodiment may be used as a display unit of a mobile terminal. In this case, the display apparatus may have both a display function and an operating function. Examples of the mobile terminal include cellular phones such as smart phones, tablets, and head mount displays.

The display apparatus according to this embodiment may be used as a display unit of an image pickup apparatus that includes an optical unit including a plurality of lenses and an image pickup element configured to receive light that has passed through the optical unit. The image pickup apparatus may include a display unit configured to display information acquired by the image pickup element. The display unit may be exposed to the outside of the image pickup apparatus or disposed in a viewfinder. The image pickup apparatus may be a digital camera or a digital camcorder.

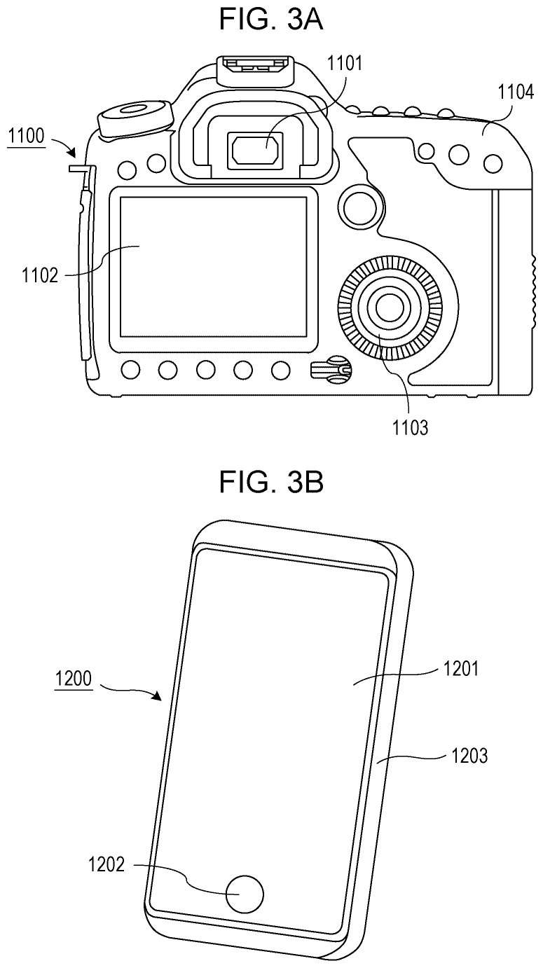

FIG. 3A is a schematic view showing an example of an image pickup apparatus according to this embodiment. An image pickup apparatus 1100 includes a viewfinder 1101, a rear display 1102, an operation unit 1103, and a housing 1104. The viewfinder 1101 may include the display apparatus according to this embodiment. In this case, the display apparatus may display not only an image to be captured but also environmental information, image capture instructions, etc. The environmental information may be, for example, the intensity of external light, the direction of external light, the moving speed of a subject, and the possibility that the subject is hidden by an object.

Since the timing appropriate for capturing an image is only a moment, the information is preferably displayed as quickly as possible. Thus, the display apparatus including the organic light-emitting element according to this embodiment is preferably used. This is because the organic light-emitting element has a high response speed. The display apparatus including the organic light-emitting element is more suitable for use in such an apparatus that requires speedy display than liquid crystal display apparatuses.

The image pickup apparatus 1100 includes an optical unit (not illustrated). The optical unit includes a plurality of lenses and focuses an image on the image pickup element accommodated in the housing 1104. By adjusting the relative positions of the plurality of lenses, the focal point can be adjusted. This operation can also be performed automatically. The image pickup apparatus may be referred to as a photoelectric conversion apparatus. Instead of sequential imaging, the photoelectric conversion apparatus may involve, as an imaging method, detection of a difference from the previous image, extraction from continuously recorded images, or the like.

FIG. 3B is a schematic view showing an example of an electronic device according to this embodiment. An electronic device 1200 includes a display unit 1201, an operation unit 1202, and a housing 1203. The housing 1203 may include a circuit, a printed board including the circuit, a battery, and a communication unit. The operation unit may be a button or a touch-sensitive response unit. The operation unit may be a biometric recognition unit that, for example, releases a lock upon recognition of fingerprints. An electronic device including a communication unit can also be referred to as a communication device. The electronic device may further has a camera function by including lenses and an image pickup element. An image captured by the camera function is displayed on the display unit. Examples of the electronic device include smartphones and notebook computers.

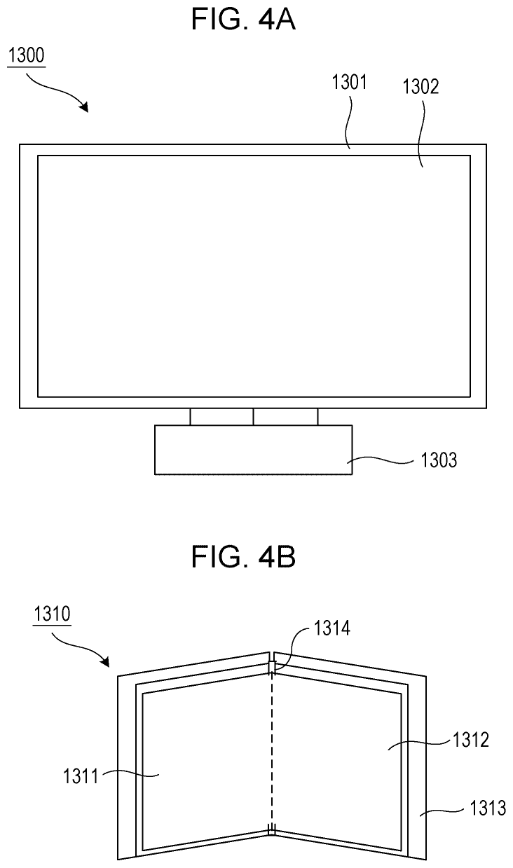

FIG. 4A and FIG. 4B show schematic views showing examples of the display apparatus according to this embodiment. FIG. 4A is a display apparatus such as a television monitor or a PC monitor. A display apparatus 1300 includes a frame 1301 and a display unit 1302. The light-emitting apparatus according to this embodiment may be used in the display unit 1302.

The display apparatus 1300 includes a base 1303 that supports the frame 1301 and the display unit 1302. The base 1303 need not necessarily be in the form illustrated in FIG. 4A. The lower side of the frame 1301 may serve as a base.

The frame 1301 and the display unit 1302 may be curved. The radius of curvature may be 5000 mm or more and 6000 mm or less.

FIG. 4B is a schematic view showing another example of the display apparatus according to this embodiment. A display apparatus 1310 in FIG. 4B is configured to be folded and what is called a foldable display apparatus. The display apparatus 1310 includes a first display unit 1311, a second display unit 1312, a housing 1313, and a bending point 1314. The first display unit 1311 and the second display unit 1312 may include the light-emitting apparatus according to this embodiment. The first display unit 1311 and the second display unit 1312 may be a seamless, monolithic display apparatus. The first display unit 1311 and the second display unit 1312 can be divided by the bending point. The first display unit 1311 and the second display unit 1312 may display different images, or the first and second display units may together display a single image.

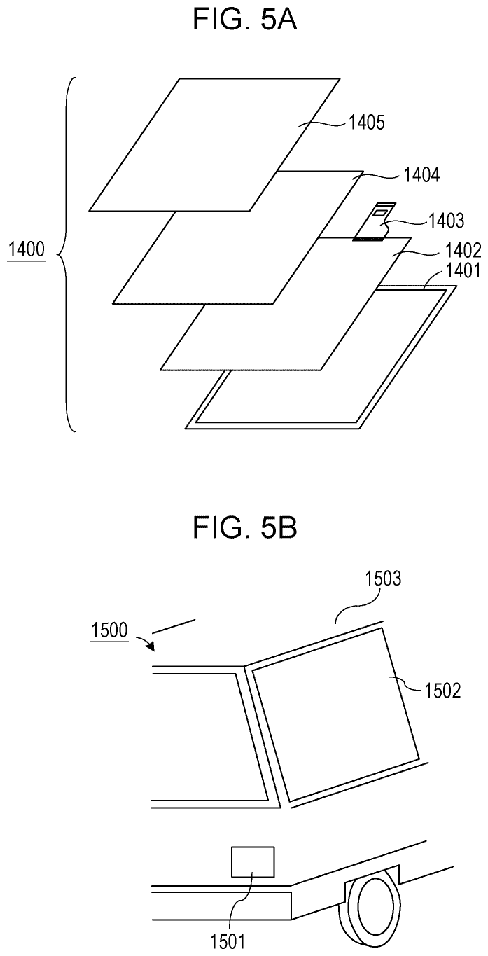

FIG. 5A is a schematic view showing an example of a lighting apparatus according to this embodiment. A lighting apparatus 1400 includes a housing 1401, a light source 1402, a circuit board 1403, an optical filter 1404, and a light diffusion unit 1405. The light source 1402 includes the organic light-emitting element according to this embodiment. The optical filter 1404 may be a filter for improving the color rendering properties of the light source. The light diffusion unit 1405 effectively diffuses light from the light source 1402 and enables the light to reach a wide region for, for example, lighting up. The optical filter 1404 and the light diffusion unit 1405 may be disposed on the light-emitting side of the lighting apparatus. If necessary, a cover may be disposed at an outermost portion.

The lighting apparatus is, for example, an indoor lighting apparatus. The lighting apparatus may emit light of white, daylight white, or any other color from blue to red. The lighting apparatus may include a light modulation circuit configured to modulate the light. The lighting apparatus includes the organic light-emitting element according to this embodiment and a power supply circuit connected thereto. The power supply circuit is a circuit configured to convert AC voltage to DC voltage. White is a color with a color temperature of 4200 K, and daylight white is a color with a color temperature of 5000 K. The lighting apparatus may include a color filter.

The lighting apparatus according to this embodiment may also include a heat dissipation unit. The heat dissipation unit dissipates heat in the apparatus to the outside and is formed of, for example, a metal with high specific heat or liquid silicone.

FIG. 5B is a schematic view of an automobile that is an example of a moving object according to this embodiment. The automobile includes a tail lamp that is an example of a lighting fixture. An automobile 1500 includes a tail lamp 1501, and the tail lamp may be configured to be turned on in response to, for example, brake operation.

The tail lamp 1501 includes the organic light-emitting element according to this embodiment. The tail lamp may include a protective member that protects the organic light-emitting element. The protective member may be made of any material that has a certain degree of high strength and is transparent, but is preferably made of a polycarbonate or the like. The polycarbonate may be mixed with a furandicarboxylic acid derivative, an acrylonitrile derivative, or the like.

The automobile 1500 may include a car body 1503 and a window 1502 attached thereto. The window may be a transparent display unless it is a window for checking the front and rear of the automobile. The transparent display may include the organic light-emitting element according to this embodiment. In this case, components of the organic light-emitting element, such as electrodes, are constituted by transparent members.

The moving object according to this embodiment may be, for example, a ship, an aircraft, or a drone. The moving object includes a body and a lighting fixture disposed on the body. The lighting fixture emits light for allowing the position of the body to be recognized. The lighting fixture includes the organic light-emitting element according to this embodiment.

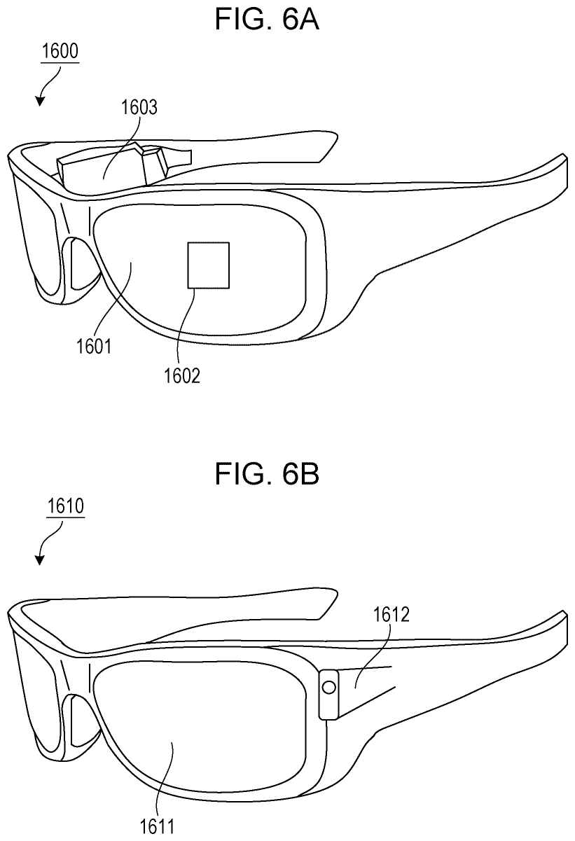

Application examples of the display apparatuses according to the above-described embodiments will be described with reference to FIG. 6A and FIG. 6B. The display apparatuses can be applied to systems that can be worn as wearable devices such as smart glasses, HMDs, and smart contact lenses. An image pickup and display apparatus used in such an application example includes an image pickup apparatus that can photoelectrically convert visible light and a display apparatus that can emit visible light.

FIG. 6A illustrates eyeglasses 1600 (smart glasses) according to one application example. An image pickup apparatus 1602, such as a CMOS sensor or a SPAD, is disposed on the front side of a lens 1601 of the eyeglasses 1600. The display apparatus according to any of the above-described embodiments is disposed on the rear side of the lens 1601.

The eyeglasses 1600 further include a controller 1603. The controller 1603 functions as a power source for supplying electricity to the image pickup apparatus 1602 and the display apparatus according to any of the embodiments. The controller 1603 controls the operation of the image pickup apparatus 1602 and the display apparatus. The lens 1601 is provided with an optical system for focusing light on the image pickup apparatus 1602.

FIG. 6B illustrates eyeglasses 1610 (smart glasses) according to one application example. The eyeglasses 1610 include a controller 1612, and the controller 1612 is equipped with an image pickup apparatus corresponding to the image pickup apparatus 1602 in FIG. 6A and a display apparatus. A lens 1611 is provided with the image pickup apparatus in the controller 1612 and an optical system for projecting light emitted from the display apparatus, and an image is projected onto the lens 1611. The controller 1612 functions as a power source for supplying electricity to the image pickup apparatus and the display apparatus and also controls the operation of the image pickup apparatus and the display apparatus. The controller may include a gaze detection unit configured to detect the gaze of a wearer. The gaze may be detected using infrared radiation. An infrared light emission unit emits infrared light to an eyeball of a user gazing at a displayed image. The reflection of the emitted infrared light from the eyeball is detected by an image pickup unit including a light-receiving element, whereby a captured image of the eyeball is obtained. Due to the presence of a reduction unit configured to reduce light from the infrared light emission unit to the display unit in plan view, degradation of image quality is reduced.

The gaze of the user toward the displayed image is detected from the captured image of the eyeball obtained by infrared imaging. Any known method can be used for the gaze detection using the captured image of the eyeball. For example, a gaze detection method based on a Purkinje image formed by the reflection of irradiation light on a cornea can be used.

More specifically, a gaze detection process based on a pupil-corneal reflection method is performed. Using the pupil-corneal reflection method, a gaze vector representing the direction (rotation angle) of the eyeball is calculated on the basis of a pupil image and a Purkinje image included in the captured image of the eyeball, whereby the gaze of the user is detected.

The display apparatus according to this embodiment may include an image pickup apparatus including a light-receiving element and may control a displayed image on the display apparatus on the basis of the gaze information of the user from the image pickup apparatus.

Specifically, the display apparatus determines, on the basis of the gaze information, a first viewing region at which the user gazes and a second viewing region other than the first viewing region. The first viewing region and the second viewing region may be determined by the controller of the display apparatus, or may be determined by an external controller and sent therefrom. In a display region of the display apparatus, the display resolution of the first viewing region may be controlled to be higher than the display resolution in the second viewing region. That is, the resolution in the second viewing region may be set to be lower than that in the first viewing region.

The display region includes a first display region and a second display region different from the first display region, and a region of high priority is determined from the first display region and the second display region on the basis of the gaze information. The first viewing region and the second viewing region may be determined by the controller of the display apparatus, or may be determined by an external controller and sent therefrom. The resolution in the region of high priority may be controlled to be higher than the resolution in regions other than the region of high priority. That is, the resolution in the regions of relatively low priority may be set to be lower.

AI may be used to determine the first viewing region or the region of high priority. AI may be a model configured to use, as teaching data, an image of an eyeball and the actual gaze direction of the eyeball in the image and estimate, from the image of the eyeball, the angle of gaze and the distance to an object gazed. The AI program may be included in the display apparatus, the image pickup apparatus, or an external apparatus. In the case of an external apparatus, transmission to the display apparatus via communications is carried out.

When display control is performed on the basis of detection by visual recognition, the display apparatus is suitable for use in smart glasses further including an image pickup apparatus configured to capture an external image. Smart glasses can display captured external information in real time.

As described above, the use of an apparatus including the organic light-emitting element according to this embodiment enables a stable display with good image quality over a long period of time.

### Included Features

The disclosure according to this embodiment includes the following features.

### Feature 1

An organometallic complex represented by general formula [1] below.

MLₘL′ₙL″ₚ [1]

In general formula [1], M is a transition metal, and L, L′, and L″ represent ligands that are different from each other.

m is an integer of 1 to 3, n is an integer of 0 to 2, and p is an integer of 0 to 2, provided that m+n+p=3. MLₘ is represented by general formula [2] below.

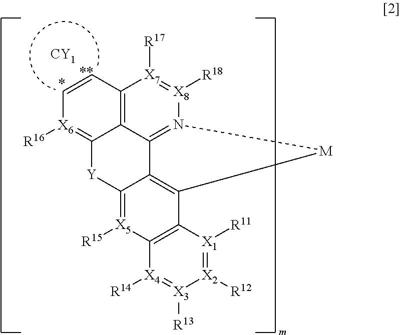

In general formula [2], Ru to R₁₈ are each independently selected from a hydrogen atom, a deuterium atom, a halogen atom, a substituted or unsubstituted alkyl group, a substituted or unsubstituted alkoxy group, a substituted or unsubstituted aryl group, a substituted or unsubstituted aryloxy group, a substituted or unsubstituted heterocyclic group, a substituted or unsubstituted aralkyl group, a substituted or unsubstituted amino group, a silyl group, and a cyano group.

Y is selected from CRR′, SiRR′, S, SO, SO₂, NR, O, Se, PRR′, PO, and SeO₂, where R and R′ are each independently selected from a substituted or unsubstituted alkyl group, a substituted or unsubstituted aryl group, and a halogen atom.

X₁ to X₈ are each independently selected from a carbon atom and a nitrogen atom, and when X₁ to X₈ are carbon atoms, the carbon atoms may be substituted with an alkyl group or an aryl group. When two adjacent X's among X₁ to X₈ are carbon atoms, the two adjacent carbon atoms may be bonded to each other to form a ring structure.

* and ** represent a position of bonding to a ring structure CY₁.

CY₁ is represented by any of general formulas [3-1] to [3-3] below.

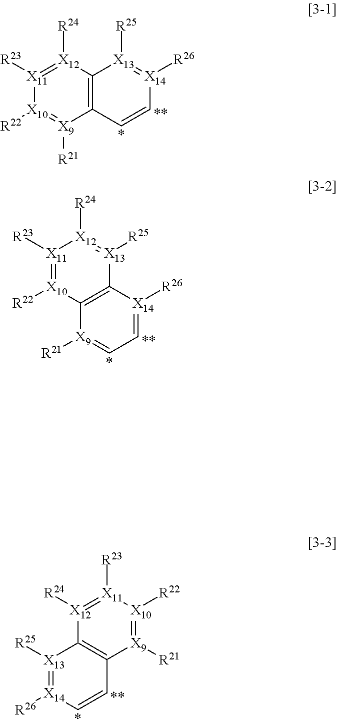

In general formulas [3-1] to [3-3], R₂₁ to R₂₆ are each independently selected from a hydrogen atom, a deuterium atom, a halogen atom, a substituted or unsubstituted alkyl group, a substituted or unsubstituted alkoxy group, a substituted or unsubstituted aryl group, a substituted or unsubstituted aryloxy group, a substituted or unsubstituted heterocyclic group, a substituted or unsubstituted aralkyl group, a substituted or unsubstituted amino group, a silyl group, and a cyano group.

X₉ to X₁₄ are each independently selected from a carbon atom and a nitrogen atom, and when X₉ to X₁₄ are carbon atoms, the carbon atoms may be substituted with an alkyl group or an aryl group. When two adjacent X's among X₉ to X₁₄ are carbon atoms, the two adjacent carbon atoms may be bonded to each other to form a ring structure.

* and ** represent a position of bonding to an isoquinoline ring.

ML′ₙ and ML″ₚ are each independently selected from general formulas [4] and [5] below.

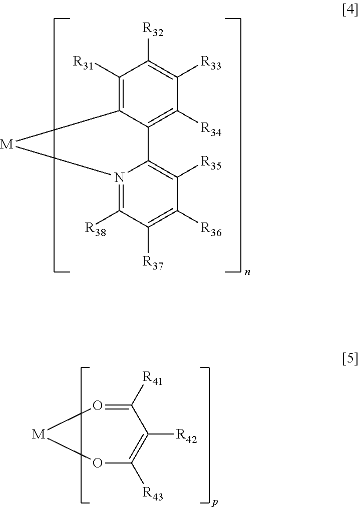

In general formulas [4] and [5], R₃₁ to R₃₈ and R₄₁ to R₄₃ are each independently selected from a hydrogen atom, a deuterium atom, a halogen atom, a substituted or unsubstituted alkyl group, a substituted or unsubstituted alkoxy group, a substituted or unsubstituted aryloxy group, a substituted or unsubstituted aralkyl group, a substituted or unsubstituted amino group, a substituted or unsubstituted aryl group, and a substituted or unsubstituted heterocyclic group.

### Feature 2

The organometallic complex according to Feature 1, wherein in general formula [2], CY₁ is represented by general formula [3-1], and Y is selected from CRR′, SiRR′, S, SO, SO₂, NR, 0, and Se, where R and R′ are each independently selected from a substituted or unsubstituted alkyl group, a substituted or unsubstituted aryl group, and a halogen atom.

### Feature 3

The organometallic complex according to Feature 1 or 2, wherein in general formula [1], M is Ir.

### Feature 4

The organometallic complex according to any one of Features 1 to 3, wherein in general formula [1], Y is CRR′, where R and R′ are each independently selected from a substituted or unsubstituted alkyl group, a substituted or unsubstituted aryl group, and a halogen atom.

### Feature 5

The organometallic complex according to any one of Features 1 to 4, wherein in general formula [1], X₁ to X₁₄ are each a carbon atom.

### Feature 6

The organometallic complex according to any one of Features 1 to 4, wherein in general formula [1], X₅ is a nitrogen atom.

### Feature 7

The organometallic complex according to any one of Features 1 to 4 and 6, wherein in general formula [1], X₁₁ is a nitrogen atom.

### Feature 8

The organometallic complex according to any one of Features 1 to 7, having an emission spectrum with a full width at half maximum of 35 nm or less.

### Feature 9

The organometallic complex according to any one of Features 1 to 8, wherein in general formula [1], R₁₁ to R₁₈ are each independently selected from a hydrogen atom, a deuterium atom, and an aryl group.

### Feature 10

A light-emitting ink composition containing: the organometallic complex according to any one of Features 1 to 9; and a solvent.

### Feature 11

An organic light-emitting element including: a first electrode; a second electrode; and an organic compound layer disposed between the first electrode and the second electrode, wherein the organic compound layer contains the organometallic complex according to any one of Features 1 to 9.

### Feature 12

The organic light-emitting element according to Feature 11, wherein the organic compound layer includes a light-emitting layer, and the light-emitting layer contains the organometallic complex and a first organic compound having a lowest excited singlet energy and a lowest excited triplet energy higher than those of the organometallic complex.

### Feature 13

The organic light-emitting element according to Feature 12, wherein the light-emitting layer contains a second organic compound different from the first organic compound, and the second organic compound has a lowest excited triplet energy that is lower than the lowest excited triplet energy of the first organic compound and higher than the lowest excited triplet energy of the organometallic complex.

### Feature 14

The organic light-emitting element according to Feature 12 or 13, wherein the organic compound layer includes, between the light-emitting layer and the second electrode, a layer formed of an organic compound, the layer having a lowest excited triplet energy higher than that of the light-emitting layer.

### Feature 15

The organic light-emitting element according to Feature 14, wherein the layer formed of an organic compound is formed of a hydrocarbon compound.

### Feature 16

The organic light-emitting element according to any one of Features 12 to 15, wherein the organic compound layer includes, between the light-emitting layer and the first electrode, a layer formed of an organic compound, the layer having a lowest excited triplet energy higher than that of the light-emitting layer.

### Feature 17

The organic light-emitting element according to Feature 16, wherein the layer formed of an organic compound is formed of a hydrocarbon compound.

### Feature 18

A display apparatus including a plurality of pixels, wherein at least one of the plurality of pixels includes the organic light-emitting element according to any one of Features 11 to 17 and a transistor connected to the organic light-emitting element.

### Feature 19

An image pickup apparatus including: an optical unit including a plurality of lenses; an image pickup element configured to receive light that has passed through the optical unit; and a display unit configured to display an image captured by the image pickup element, wherein the display unit includes the organic light-emitting element according to any one of Features 11 to 17.

### Feature 20

An electronic device including: a display unit including the organic light-emitting element according to any one of Features 11 to 17; a housing provided with the display unit; and a communication unit provided in the housing and configured to communicate with an external device.

### Feature 21

A lighting apparatus including: a light source including the organic light-emitting element according to any one of Features 11 to 17; and a light diffusion unit or an optical filter configured to transmit light emitted from the light source.

### Feature 22

A moving object including: a lighting fixture including the organic light-emitting element according to any one of Features 11 to 17; and a body provided with the lighting fixture.

### Feature 23

An image forming apparatus including: a photoreceptor; and an exposure light source configured to expose the photoreceptor, wherein the exposure light source includes the organic light-emitting element according to any one of Features 11 to 17.

### EXAMPLES

The present invention will now be described more specifically with reference to examples, but the present invention is not limited thereto.

### Example 1

The organometallic complex according to this embodiment can be synthesized by, for example, the following synthesis method. In the following, a method of synthesizing exemplary compound 102 is given as an example.

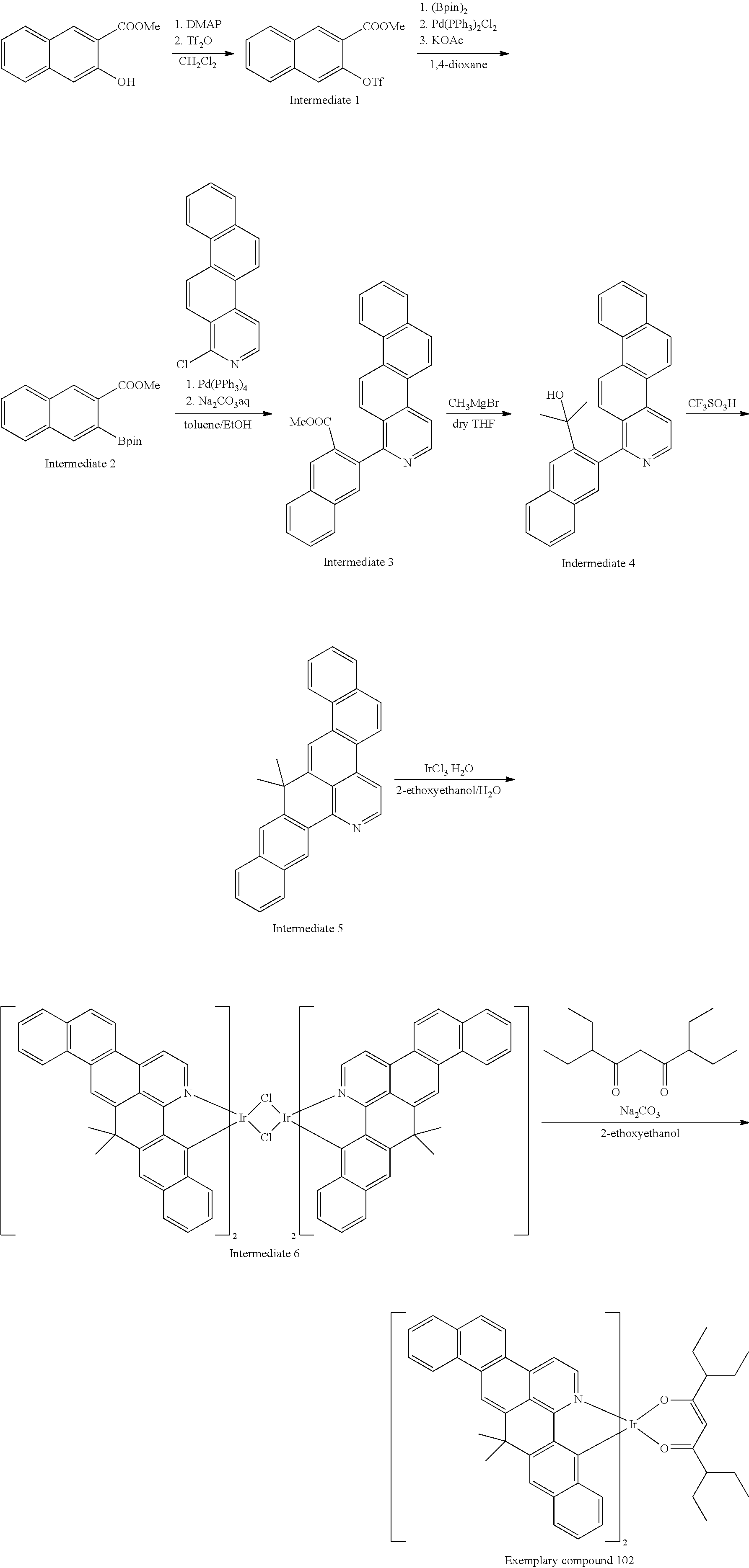

DMAP: 4-dimethylaminopyridine
Tf₂O: trifluoromethanesulfonic anhydride
(Bpin)₂: bis(pinacolato)diboron
Pd(PPh₃)₂Cl₂: bis(triphenylphosphine)palladium(II) dichloride
KOAc: potassium acetate
Pd(PPh₃)₄: tetrakis(triphenylphosphine)palladium(0)

The synthesis method will be described in detail below, but the present invention is not limited thereto.

### Synthesis of Intermediate 1

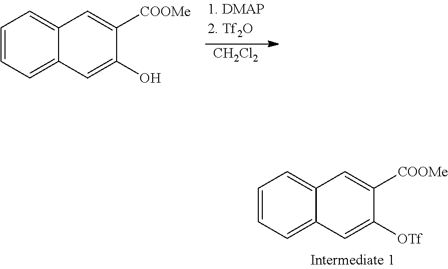 [SMILES: COC(=O)c1cc2ccccc2cc1O ; "CH2Cl2" ; COC(=O)c1cc2ccccc2cc1OS(=O)(=O)C(F)(F)F]

In a nitrogen atmosphere, methyl 3-hydroxy-2-naphthoate (0.50 g, 2.47 mmol) and 4-dimethylaminopyridine (0.60 g, 4.94 mmol) were dissolved in 15 mL of CH₂Cl₂, and the resultant was stirred in an ice bath. Trifluoromethanesulfonic anhydride (1.40 g, 4.96 mmol) was added dropwise through an isobaric dropping funnel over 10 minutes, and the resultant was stirred for 3 hours while being kept in the ice bath. A white solid precipitated as the reaction proceeded.

To the reaction solution, 10 mL of a 0.5 M aqueous hydrochloric acid solution was slowly added to perform liquid-liquid separation, and the organic layer was washed. This operation was repeated three times.

Upon concentration of the organic layer, a white solid precipitated, and thus the white solid was collected by filtration while being washed with MeOH to obtain 0.80 g of intermediate 1. The yield was 97%.

### Synthesis of Intermediate 2

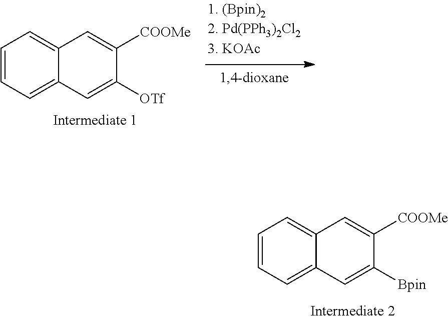

In a nitrogen atmosphere, intermediate 1 (0.80 g, 2.39 mmol), KOAc (0.70 g, 7.18 mmol), (Bpin)₂ (1.82 g, 7.18 mmol), and Pd(PPh₃)₂Cl₂ (0.084 g, 0.12 mmol) were dissolved in 15 mL of super-dehydrated 1,4-dioxane, and the resultant was stirred with heating at 110° C. for 3 hours.

The reaction solution was allowed to cool and subjected to Celite filtration while being washed with a small amount of dioxane, and the resulting filtrate was concentrated to obtain a black oily substance. This was stirred in an ice bath while being washed with a small amount of MeOH, as a result of which a gray solid precipitated and thus was collected by filtration to obtain 0.72 g of intermediate 2. The yield was 96%.

### Synthesis of Intermediate 3

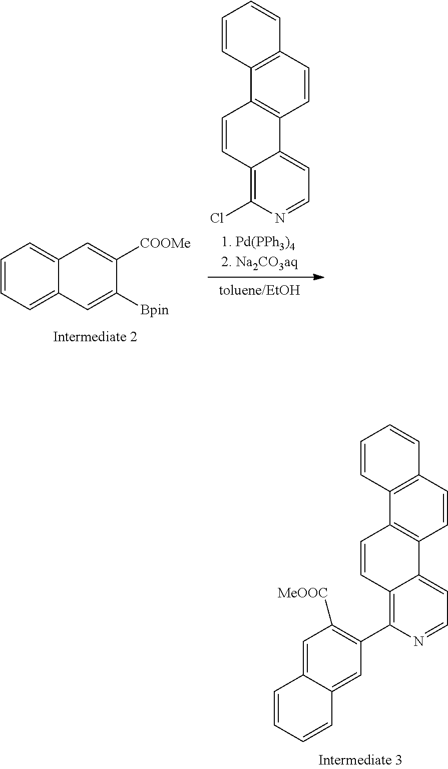

In a nitrogen atmosphere, intermediate 2 (0.3 g, 0.96 mmol), 1-chloronaphtho[2,1-f]isoquinoline (0.17 g, 0.64 mmol), Na₂CO₃ (0.20 g, 1.89 mmol), and Pd(PPh₃)₄ (40 mg, 34.6 μmol) were dissolved in a mixed solution of 15 mL of toluene, 1 mL of EtOH, and 0.5 mL of H₂O, and the resultant was stirred with heating at 70° C. for 5 hours. The reaction solution was allowed to cool, and subjected to liquid-liquid separation using chloroform and water to extract an organic layer. The organic layer was concentrated to obtain a brown oily substance. This was purified by silica gel column chromatography (eluent: toluene to 20% ethyl acetate/toluene) to obtain 50 mg of intermediate 3. The yield was 19%.

### Synthesis of Intermediate 4

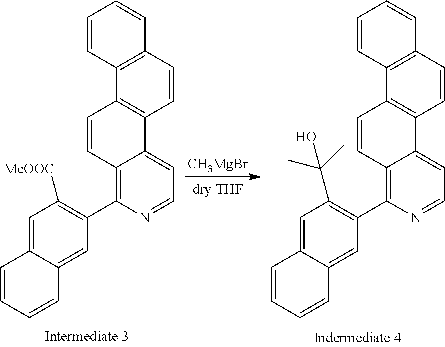

In a nitrogen atmosphere, intermediate 3 (50 mg, 0.12 mmol) was dissolved in 5 mL of super-dehydrated THF, and the resultant was stirred in an ice bath. To the reaction solution, a 1.0 M CH₃MgBr/THF solution (1.0 mL, 1.0 mmol) was added, and the resultant was stirred for 12 hours. A saturated aqueous NH₄Cl solution was slowly added dropwise to terminate the reaction. Liquid-liquid separation was performed using chloroform and water to extract an organic layer. The organic layer was concentrated to obtain 45 mg of a brown oily substance as intermediate 4. The yield was 90%.

### Synthesis of Intermediate 5

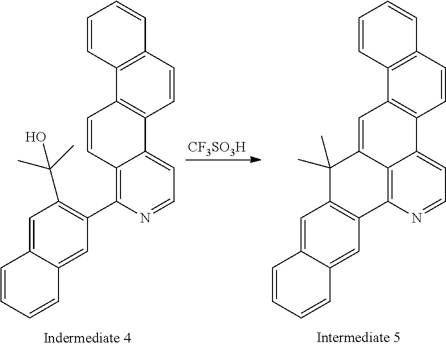

In a nitrogen atmosphere, intermediate 4 (45 mg, 0.11 mmol) was dissolved in 2 mL of trifluoromethanesulfonic acid, and the resultant was stirred with heating at 130° C. for 5 hours. The reaction solution was allowed to cool and neutralized with an aqueous NaOH solution in an ice bath, and then an organic layer was extracted with chloroform. The organic layer was concentrated to obtain a brown oily substance. This was purified by silica gel column chromatography (eluent: toluene) to obtain 20 mg of intermediate 5. The yield was 47%.

### Synthesis of Intermediate 6

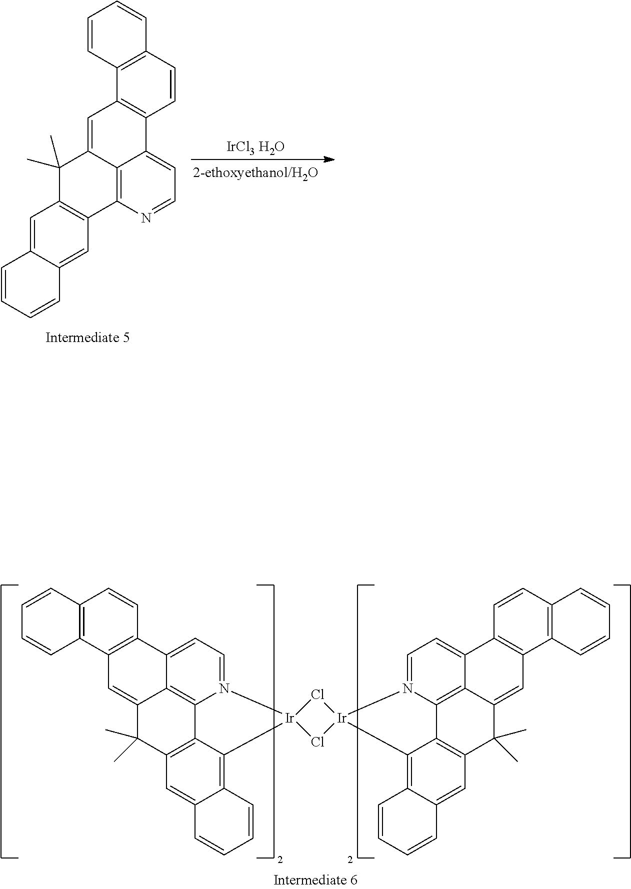

In a nitrogen atmosphere, intermediate 5 (20 mg, 51 μmol) and IrCl₃·H₂O (7.1 mg, 22.4 μmol) were dissolved in a mixed solution of 2 mL of 2-ethoxyethanol and 1 mL of H₂O, and the resultant was stirred with heating at 120° C. for 12 hours. The reaction solution was allowed to cool, and a precipitated red solid was collected by filtration to obtain 15 mg of intermediate 6. The yield was 73%.

### Synthesis of Exemplary Compound 102

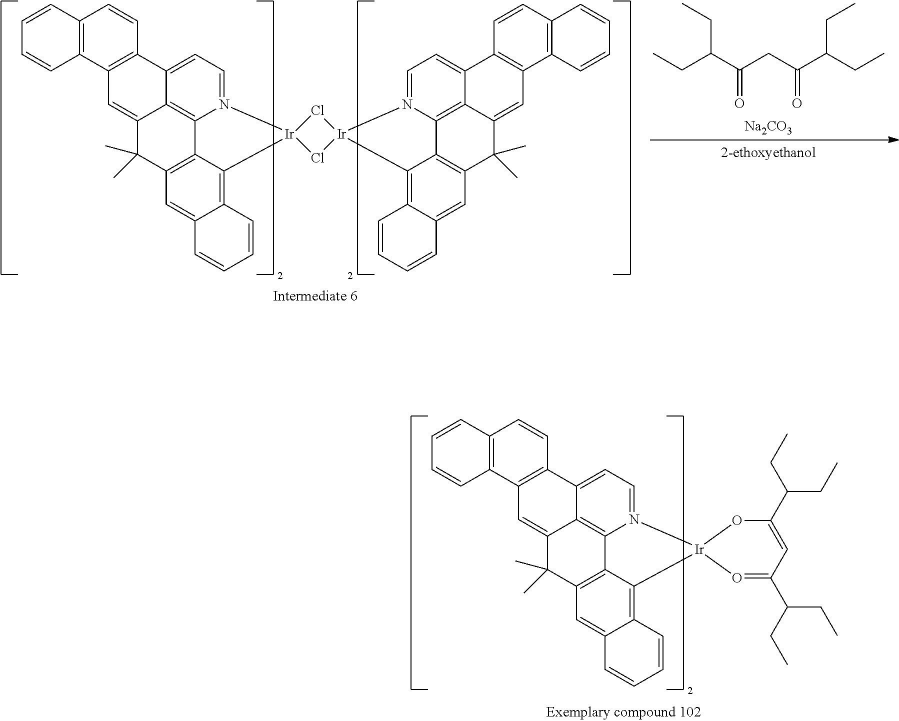

In a nitrogen atmosphere, intermediate 6 (15 mg, 7.4 μmol), Na₂CO₃ (5 mg, 47 μmol), and 3,7-diethylnonane-4,6-dione (10 mg, 47 μmol) were dissolved in 0.5 mL of 2-ethoxyethanol, and the resultant was stirred with heating at 120° C. for 12 hours. The reaction solution was allowed to cool, and 0.5 mL of H₂O was added. A precipitated red solid was collected by filtration to obtain 5 mg of exemplary compound 102. The yield was 28%.

### Examples 2 to 12 and Comparative Example 1 to Comparative Example 3

Organometallic complexes according to this embodiment and compound A to compound C listed in Table 1 above were synthesized in the same manner as in Example 1.

### Optical Properties Evaluation

The organometallic complexes of Examples 1 to 12 and Comparative Examples 1 to 3 were each dissolved in toluene at a concentration of 1.0×10⁻⁵ M, and after the solutions were each bubbled with N₂ for 5 minutes, the FWHM of an emission spectrum and the emission quantum yield were measured, and values relative to the FWHM and the emission quantum yield of compound B each taken as 1 were calculated. The results are shown in Table 2.

TABLE 2

| | Organometallic | | Emission quantum |
|---|---|---|---|
| | complex | FWHM | yield |
| Example 1 | Exemplary | 0.82 | 1.37 |
| | compound 102 | | |
| Example 2 | Exemplary | 0.88 | 1.42 |
| | compound 2 | | |
| Example 3 | Exemplary | 0.82 | 1.45 |
| | compound 19 | | |
| Example 4 | Exemplary | 0.91 | 1.35 |
| | compound 30 | | |
| Example 5 | Exemplary | 0.97 | 1.23 |
| | compound 75 | | |
| Example 6 | Exemplary | 0.85 | 1.38 |
| | compound 100 | | |
| Example 7 | Exemplary | 0.85 | 1.42 |
| | compound 111 | | |
| Example 8 | Exemplary | 0.91 | 1.32 |
| | compound 114 | | |
| Example 9 | Exemplary | 1.00 | 1.28 |
| | compound 155 | | |
| Example 10 | Exemplary | 0.94 | 1.40 |
| | compound 201 | | |
| Example 11 | Exemplary | 1.00 | 1.31 |
| | compound 225 | | |
| Example 12 | Exemplary | 0.88 | 1.38 |
| | compound 258 | | |
| Comparative | Compound A | 1.97 | 1.25 |
| Example 1 | (Table 1) | | |
| Comparative | Compound B | 1.00 | 1.00 |
| Example 2 | (Table 1) | | |
| Comparative | Compound C | 0.97 | 1.08 |
| Example 3 | (Table 1) | | |

As shown in Table 2, as compared with the organometallic complexes of Comparative Examples, the organometallic complexes of Examples, each having a NIQ ring, were superior in both the FWHM and the emission quantum yield or greatly improved in one of them, if inferior in the other, and had excellent optical properties as a whole. In particular, exemplary compound 102 had the smallest FWHM and the high emission quantum yield.

According to the present invention, an organometallic complex having an emission spectrum with a small FWHM and having high light-emission efficiency can be provided.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An organometallic complex represented by formula [1]:
MLₘL′ₙL″ₚ [1]
where M is a transition metal, and L, L′, and L″ represent ligands that are different from each other,
m is an integer of 1 to 3, n is an integer of 0 to 2, p is an integer of 0 to 2, provided that m+n+p=3, and MLₘ is represented by formula [2]: 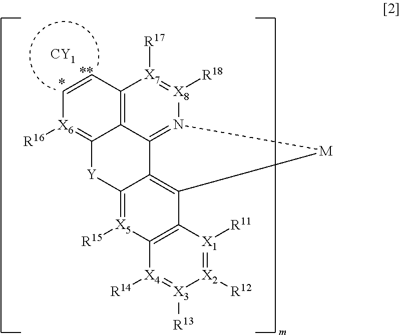
where R₁₁ to R₁₈ are each independently selected from a hydrogen atom, a deuterium atom, a halogen atom, a substituted or unsubstituted alkyl group, a substituted or unsubstituted alkoxy group, a substituted or unsubstituted aryl group, a substituted or unsubstituted aryloxy group, a substituted or unsubstituted heterocyclic group, a substituted or unsubstituted aralkyl group, a substituted or unsubstituted amino group, a silyl group, and a cyano group,
Y is selected from CRR′, SiRR′, S, SO, SO₂, NR, O, Se, PRR′, PO, and SeO₂, where R and R′ are each independently selected from a substituted or unsubstituted alkyl group, a substituted or unsubstituted aryl group, and a halogen atom,
X₁ to X₈ are each independently selected from a carbon atom and a nitrogen atom, and when X₁ to X₈ are carbon atoms, the carbon atoms may be substituted with an alkyl group or an aryl group; when two adjacent X's among X₁ to X₈ are carbon atoms, the two adjacent carbon atoms may be bonded to each other to form a ring structure,
* and ** represent a position of bonding to a ring structure CY₁, and
CY₁ is represented by any of formulas [3-1] to [3-3]: 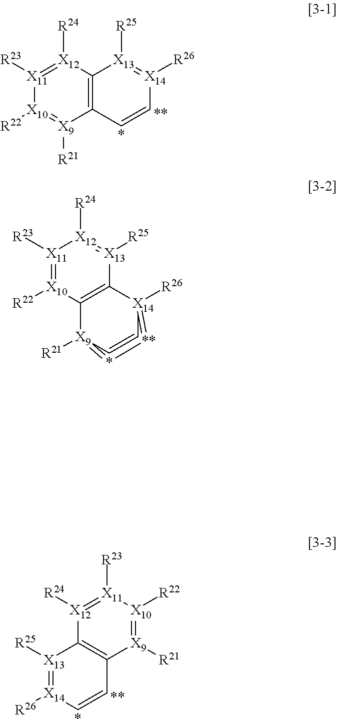
where R₂₁ to R₂₆ are each independently selected from a hydrogen atom, a deuterium atom, a halogen atom, a substituted or unsubstituted alkyl group, a substituted or unsubstituted alkoxy group, a substituted or unsubstituted aryl group, a substituted or unsubstituted aryloxy group, a substituted or unsubstituted heterocyclic group, a substituted or unsubstituted aralkyl group, a substituted or unsubstituted amino group, a silyl group, and a cyano group,
X₉ to X₁₄ are each independently selected from a carbon atom and a nitrogen atom, and when X₉ to X₁₄ are carbon atoms, the carbon atoms may be substituted with an alkyl group or an aryl group; when two adjacent X's among X₉ to X₁₄ are carbon atoms, the two adjacent carbon atoms may be bonded to each other to form a ring structure,
* and ** represent a position of bonding to an isoquinoline ring, and
ML′ₙ and ML″ₚ are each independently selected from formulas [4] and [5]: 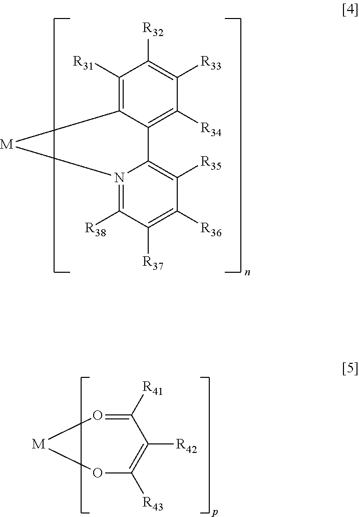
where R₃₁ to R₃₈ and R₄₁ to R₄₃ are each independently selected from a hydrogen atom, a deuterium atom, a halogen atom, a substituted or unsubstituted alkyl group, a substituted or unsubstituted alkoxy group, a substituted or unsubstituted aryloxy group, a substituted or unsubstituted aralkyl group, a substituted or unsubstituted amino group, a substituted or unsubstituted aryl group, and a substituted or unsubstituted heterocyclic group.

2. The organometallic complex according to claim 1, wherein in formula [2], CY₁ is represented by formula [3-1], and Y is selected from CRR′, SiRR′, S, SO, SO₂, NR, O, and Se, where R and R′ are each independently selected from a substituted or unsubstituted alkyl group, a substituted or unsubstituted aryl group, and a halogen atom.

3. The organometallic complex according to claim 1, wherein in formula [1], M is Ir.

4. The organometallic complex according to claim 1, wherein in formula [2], Y is CRR′, where R and R′ are each independently selected from a substituted or unsubstituted alkyl group, a substituted or unsubstituted aryl group, and a halogen atom.

5. The organometallic complex according to claim 1, wherein in formula [2], Y is selected from CRR′, SiRR′, S, SO, SO₂, NR, and Se, where R and R′ are each independently selected from a substituted or unsubstituted alkyl group, a substituted or unsubstituted aryl group, and a halogen atom.

6. The organometallic complex according to claim 1, wherein in formula [1], X₁ to X₁₄ are each a carbon atom.

7. The organometallic complex according to claim 1, wherein in formula [1], X₅ is a nitrogen atom.

8. The organometallic complex according to claim 1, wherein in formula [1], X₁₁ is a nitrogen atom.

9. The organometallic complex according to claim 1, having an emission spectrum with a full width at half maximum of 35 nm or less.

10. The organometallic complex according to claim 1, wherein in formula [1], R₁₁ to R₁₈ are each independently selected from a hydrogen atom, a deuterium atom, and an aryl group.

11. A light-emitting ink composition comprising:
the organometallic complex according to claim 1; and
a solvent.

12. An organic light-emitting element comprising:
a first electrode;
a second electrode; and
an organic compound layer disposed between the first electrode and the second electrode,
wherein the organic compound layer contains the organometallic complex according to claim 1.

13. The organic light-emitting element according to claim 12, wherein the organic compound layer includes a light-emitting layer, and
the light-emitting layer contains the organometallic complex and a first organic compound having a lowest excited singlet energy and a lowest excited triplet energy higher than those of the organometallic complex.

14. The organic light-emitting element according to claim 13, wherein the light-emitting layer contains a second organic compound different from the first organic compound, and
the second organic compound has a lowest excited triplet energy that is lower than the lowest excited triplet energy of the first organic compound and higher than the lowest excited triplet energy of the organometallic complex.

15. The organic light-emitting element according to claim 13, wherein the organic compound layer includes, between the light-emitting layer and the second electrode, a layer formed of an organic compound, the layer having a lowest excited triplet energy higher than that of the light-emitting layer.

16. The organic light-emitting element according to claim 15, wherein the layer formed of an organic compound is formed of a hydrocarbon compound.

17. The organic light-emitting element according to claim 13, wherein the organic compound layer includes, between the light-emitting layer and the first electrode, a layer formed of an organic compound, the layer having a lowest excited triplet energy higher than that of the light-emitting layer.

18. The organic light-emitting element according to claim 17, wherein the layer formed of an organic compound is formed of a hydrocarbon compound.

19. A display apparatus comprising a plurality of pixels, wherein at least one of the plurality of pixels includes the organic light-emitting element according to claim 12 and a transistor connected to the organic light-emitting element.

20. An image pickup apparatus comprising:
an optical unit including a plurality of lenses;
an image pickup element configured to receive light that has passed through the optical unit; and
a display unit configured to display an image captured by the image pickup element,
wherein the display unit includes the organic light-emitting element according to claim 12.

21. An electronic device comprising:
a display unit including the organic light-emitting element according to claim 12;
a housing provided with the display unit; and
a communication unit provided in the housing and configured to communicate with an external device.

22. A lighting apparatus comprising:
a light source including the organic light-emitting element according to claim 12; and
a light diffusion unit or an optical filter configured to transmit light emitted from the light source.

23. A moving object comprising:
a lighting fixture including the organic light-emitting element according to claim 12; and
a body provided with the lighting fixture.

24. An image forming apparatus comprising:
a photoreceptor; and
an exposure light source configured to expose the photoreceptor,
wherein the exposure light source includes the organic light-emitting element according to claim 12.
